SEQUENCE LISTING (7 sequences)

SEQ ID NO: 1 - protein, 4 aa, Artificial
  O-linked glycosylation site sequence
XXXP

SEQ ID NO: 2 - protein, 451 aa, Artificial
  amino acid sequence of anti-CD20 IgG1 mAb heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 213 aa, Artificial
  amino acid sequence of anti-CD20 IgG1 mAb light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, Artificial
  amino acid sequence of anti-CD20 IgG4 mAb heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 213 aa, Artificial; identical to SEQ ID NO: 3
  amino acid sequence of anti-CD20 IgG4 mAb light chain

SEQ ID NO: 6 - protein, 303 aa, Artificial
  amino acid sequence of MCP1-Fc fusion protein
QPDAINAPVTCCYNFTNRKISVQRLASYRRITSSKCPKEAVIFKTIVAKEICADPKQKWVQDSMDHLDKQTQTPKTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 401 aa, Artificial
  amino acid sequence of EPO-Fc fusion protein
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDREFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2012/0258496 A1
(43) Pub. Date: Oct. 11, 2012

(54) PRODUCTION OF LOW FUCOSE ANTIBODIES IN H4-II-E RAT CELLS

(75) Inventors: Kristina Ellwanger, Heidelberg (DE); Lore Florin, Biberach an der Riss (DE); Hitto Kaufmann, Ulm (DE)

(73) Assignee: BOEHRINGER INGELHEIM INTERNATIONAL GMBH, Ingelheim am Rhein (DE)

(21) Appl. No.: 13/239,654

(22) Filed: Sep. 22, 2011

(30) Foreign Application Priority Data
Sep. 27, 2010 (EP) 10 180 321.1
Mar. 3, 2011 (EP) 11 156 848.1

(51) Int. Cl. C12P 21/00; C12N 5/10; C12N 15/85

(52) U.S. Cl. 435/69.6; 435/69.1; 435/69.7; 435/455; 435/353

(57) ABSTRACT

The invention concerns the field of cell culture technology. It specifically concerns a rat hepatoma cell, preferably a H4-II-E rat hepatoma cell, carrying a DNA encoding an antibody or Fc-fusion protein and having low fucosylation activity for adding fucose to glycosidic structures such as biantennary glycans, e.g. N-acetylglucosamine. The invention furthermore concerns a method for producing low fucose glycoproteins especially antibodies or Fc-fusion proteins in rat hepatoma cells, preferably in H4-II-E rat hepatoma cells. It further concerns the identification and generation of new host cell lines which are capable of synthetizing glycoproteins with beneficial properties, improving the therapeutic efficacy and/or serum half-life of the product compared to products from commonly used host cell lines.

### BACKGROUND OF THE INVENTION

1. Technical Field

The invention concerns the field of cell culture technology. It concerns a method for producing low fucose glycoproteins especially antibodies and Fc-fusion proteins in H4-II-E rat hepatoma cells. It concerns the identification and generation of new host cell lines which are capable of synthetizing glycoproteins with beneficial properties, improving the therapeutic efficacy and/or serum half-life of the product compared to products from commonly used host cell lines. The invention concerns the use and optimization of such cell lines, particularly of the rat hepatoma cell line H4-II-E, for the expression of recombinant proteins and their application as highly active biopharmaceutical therapeutics.

2. Background

The market for biopharmaceuticals for use in human therapy continues to grow at a high rate with 270 new biopharmaceuticals being evaluated in clinical studies and estimated sales of 30 billions in 2003 (Werner, 2004). Biopharmaceuticals can be produced from various host cell systems, including bacterial cells, yeast cells, insect cells, plant cells and mammalian cells as well as human-derived cell lines. Currently, an increasing number of biopharmaceuticals is produced from eukaryotic cells due to their ability to correctly process and post-translationally modify recombinant human proteins. Therefore, a key issue affecting the choice of a cell line for use in a manufacturing process is the ability to consistently produce the product with a uniform post translational modification pattern (yielding high biological activity, stability and batch-to batch consistency).

The largest proportion of antibodies currently licensed for therapeutic use are manufactured in Chinese hamster ovary (CHO) cell lines. Other production systems are murine lymphoid cells (including NS0 and Sp2/0-Ag 14). These parental cell lines are also the ones most commonly used for the production of antibodies in clinical trials. In addition, murine and other cell lines such as the human cell line PER.C6 are used.

Therapies with monoclonal antibodies have become one of the main focus of the biotechnology industry. There are already efficient monoclonal antibodies approved by the Food and Drug Administration, but there is still a need for even more effective and compatible drugs, not only to reduce the costs for production but also to facilitate the application for more patients as until now. Furthermore, the application dose of optimized therapeutics could be diminished thus leading to a higher tolerance and less adverse effects. Among the five antibody classes IgA, IgD, IgE, IgG and IgM in mammals mainly the IgG class is used for treatment, prevention and diagnosis of various diseases. This is due to their favorable functional characteristics such as long half-life in blood and various effector functions such as antibody-dependent cell-mediated cytotoxicity (ADCC) and complement-dependent cytotoxicity (CDC). Among the human IgG class the subclasses (isotypes) IgG1 and IgG3 have the highest ADCC and CDC activity but half-life of IgG3 is only 7 to 8 days compared to IgG1 with up to 21 days. In view of the above, mostly antibodies of the human IgG1 subclass are used, if for optimal therapeutical efficacy highly active effector functions are required to remove cells carrying the antigen on its surface.

Post-translational modifications are not only crucial for correct protein folding, intracellular trafficking, solubility and stability, but also have a significant functional impact on the biological activity and immunogenicity of secreted proteins. In biopharmaceutical proteins, e.g. therapeutic antibodies, post-translational modifications can have a particular impact on the therapeutic potency, pharmacokinetics, pharmacodynamics, and immunogenicity of the product.

Glycosylation represents the most widespread post-translational modification found in natural secretory proteins as well as in approved biopharmaceuticals. Almost 50% of human proteins are glycosylated. Asparagine (N)- and Serine (O)-linked glycans are the two principle glycan classes formed on mammalian cell-derived secretory glycoproteins. The transfer of glycostructures to secreted proteins is taking place in the endoplasmatic reticulum (ER) and the Golgi apparatus and represents a complex enzymatic process, regulated by the activity of numerous genes. Defects in a number of genes involved in the glycosylation pathway cause congenital disorders with serious medical consequences, confirming the importance of correct glycosylation.

In eukaryotes, N-linked glycans are attached to proteins in the lumen of the ER as pre-synthesized oligosaccharides, consisting of branched oligosaccharide units, composed of 3 glucoses (Glc), 9 mannoses (Man) and 2 N-acetylglucosamines (GlcNAc). These core glycans (Glc₃Man₉GlcNAc₂) are transferred via a lipid carrier, the dolichol-pyrophosphate in the ER membrane to secreted proteins translocated into the ER lumen. Glycans are transferred onto appropriate sequences, namely Asn-X-Ser/Thr, where X is any amino acid, except of proline in a nascent glycoprotein. Correctly folded glycoproteins are actively transported to the Golgi apparatus where their N-glycans are modified by glucosidases, mannosidases and glycosyltransferases to yield complex, sialic acid, fucose and galactose containing structures. Glucosidases and mannosidases remove glucose (Glc) and mannose monosaccharides (Man) from glycans at the earliest stages of N-glycan processing. N-acetylglucosaminidases then catalyze the addition of N-acetylglucosamine (GlcNAc) to the mannose sugars attached to the conserved core structure of the N-glycan, having a determining role towards the number of branches or antennae, which are formed on the glycan. Fucosyltransferases add fucose to the N-acetylglucosamine proximal to the protein and galactosyltransferases and sialyltransferases add galactose and sialic acid, respectively, onto the terminal ends of the N-glycan branches. The reactions of these enzymes are generally irreversible, generating stable N-glycosylated proteins.

Through the generation and differential modification of core oligosaccharides and the variable addition of outer arm sugars, protein glycosylation introduces a considerable heterogeneity. It is proven that incorrectly glycosylated or aglycosylated antibodies display uncontrolled functions. The choice of an appropriate production system capable of consistently generating product with the desired pattern of post-translational modifications is therefore crucial for successful drug manufacturing and application. The profile of glycoforms and thereby the functional activity of a glycoprotein may differ significantly depending on the production system. Biopharmaceutical protein production in prokaryotic production systems (e.g. Escherichia coli) results in aglycosylated protein products being recovered as inclusion bodies that have to be solubilized and refolded in vitro. In contrast, yeast expression systems add sugar side chains of high mannose content, and the glycosylation pattern obtained after production in insect cells also differs significantly from characteristic mammalian patterns. Plants may differentially glycosylate proteins but consistently add α-1,3-fucose and β1,3-xylose sugars that are reported to be immunogenic or allergenic in humans.

Various mammalian host cells are capable of differential N-glycan processing. Analysis of the produced glycoprotein reveals hetergeneous glycoforms depending on tissue or species from which the production cell originates. It is therefore important to ensure that the glycosylation pattern of glycoprotein products produced for clinical use is uniform throughout and between production lots but also that favorable in vivo properties of the antibodies are retained.

Both, natural (intrinsic) antibodies in the body as well as antibodies produced by recombinant DNA technologies in mammalian host cell lines are glycosylated through the covalent attachment of oligosaccharides at an evolutionarily conserved Asn297 in the CH2 domain within the IgG-Fc region. The oligosaccharide is an integral part of the IgG-Fc structure and absolutely required for effector functions. The interaction sites on IgG-Fc for effector ligands like FcγRI, FcγRII, FcγRIII, and C1 q are comprised of the protein moiety; however, the generation of the essential IgG-Fc protein conformation is dependent on the presence and chemical constitution of the oligosaccharide. Thus, the effector mechanisms mediated through the engagement of effector ligands (clearance mechanisms, like phagocytosis, antibody-dependent cellular cytotoxicity (ADCC) and complement-dependent cellular cytotoxicity (CDC)) are severly compromised or ablated for incorrectly or non-glycosylated IgGs.

The human FcγRIIIa receptor is polymorphic and it has been shown that the FcγRIIIa-158V (valine) form has a higher affinity for IgG1-Fc than the FcγRIIIa-158F (phenylalanine) form. It was demonstrated in vitro that IgG1 antibodies more efficiently mediate ADCC through homozygous FcγRIIIa-158V bearing cells than homozygous FcγRIIIa-158F or heterozygous FcγRIIIa-158V/FcγRIIIa-158F cells.

The typical oligosaccharide structures of normal polyclonal human IgG-Fc are of the complex biantennary type. The biantennary core heptasaccharide is variably modified as the glycoproteins transit the Golgi with additional sugar residues at the core (Fucose, N-Acetylglucosamine (GlcNAc)) or outer arms (Galactose (Gal), and N-Acetylneuraminic acid (Neu5Ac)) (Review by (Walsh and Jefferis, 2006)).

Serum IgG antibodies from different vertebrate species have in common the basic biantennary Fc glycostructure, but differ in the structure and composition in the peripheral regions of the sugar chains (Hamako et al., 1993). The variability is most likely due to varied activities of glycosyltransferases present in the B-lymphocytes of different species and/or the accessibility of each IgG towards these transferases. If recombinant DNA technologies are used to stably express IgGs in host cells which do not naturally secrete IgGs, the glycosylation pattern is likewise specified by the activities of glycosyltransferases or glycan modifying enzymes present in that cell line. Accordingly, the glycosylation patterns of recombinant proteins produced in alternative production cell lines are cell type-, tissue- and species-specific and can vary significantly (Raju et al., 2000). The production of the same IgG1 antibody in CHO, Y0 myeloma and NS0 cells, gives rise to three different products, differing in their glycosylation pattern and biological activity (Lifely et al., 1995). It is therefore well established that the activation of effector functions strongly depends on the oligosaccharide composition of the antibody molecule and that the activation of certain effector functions is more effective if certain glycosylation patterns are present.

Studies employing therapeutic antibodies with a modified glycosylation pattern suggest, that the lack of α1,6-linked core fucose results in increased affinity of binding of the IgG1 antibody to the FcγRIIIa receptor, and consequently an up to 100fold increased ADCC efficacy, mediated by natural killer (NK) cells.

Due to the high affinity binding of components of the complement system to galactosylated Fc-glycans, antibodies with increased galactosylation of Fc glycans activate the complement system and complement dependent cellular cytotoxicity (CDC) more efficiently than antibodies with low or non-galactosylated glycoforms.

Therapeutic antibodies can have distinct methods of action. Some antibodies, antibody fragments, or Fc-fusion proteins are designed to neutralize biomolecules like cytokines in vivo. In contrast, recombinant antibodies in cancer therapy often recognize proteins on tumor cells and their efficacy unambiguously depends on sensitizing effector cells for subsequent killing by the mechanisms of antibody-dependent cellular cytotoxicity (ADCC) and/or complement-dependent cytotoxicity (CDC). Yet, for other antibody-based therapies, the activation of inflammatory cascades may be detrimental, giving rise to unwanted side effects.

Thus, it is a challenge, that antibodies produced in commonly used production cell lines like CHO, NS0 and SP2/0 do not show optimal Fc glycosylation patterns.

Antibodies produced in CHO, NS0 or human cell lines, show a Fc glycosylation profile comprised of mainly (˜95%) the biantennary core heptasaccharide structure carrying at the central GlcNAc a α1,6 linked fucose residue.

It is well established, that the presence of α1,6-linked fucose within the biantennary carbohydrate core structure in Fc glycans significantly impairs the potential of an antibody to activate effector functions like the antibody-dependent cellular cytotoxicity (ADCC). Another effector function, the complement-induced cytotoxicity (CDC) has been demonstrated to be galactosylation-dependent with higher content of galactose resulting in enhanced CDC activities. Thus, monoclonal antibodies with fucosylated glycans and a low degree of galactose are not particularly effective in the therapy of solid or non-solid tumors, since the therapeutic outcome of antibody therapies in this context largely depend on the antibodies potency in recruiting and activating tumor attacking immune cells, apoptosis induction or the activation of ADCC or CDC. Therefore, antibodies produced in CHO remain of limited benefit in cancer therapy, and to overcome their low activity, have to be administered to patients in high doses. In contrast, antibodies with an altered glycosylation pattern (e.g. lacking α1,6-linked core fucose) should have a significantly enhanced therapeutic efficacy. Despite the fact that most licensed therapeutic antibodies are produced in CHO, NS0 or Sp2/0 cells, it is known that under non-optimal conditions, these cells can produce a number of abnormally glycosylated products that lack potency or are potentially immunogenic. In addition to incorrectly processed oligosaccharides, murine cells are also known to add sugar residues not normally found on human IgGs. Such structure like Gal-1,3-Gal and N-glycolylneuraminic acid (NeuGc) can trigger immunogenic or allergic reactions, which is unacceptable for therapeutics to be delivered to human patients in vivo.

Up to this point, experimental approaches to obtain cell lines capable of producing proteins with advantageous glycosylation pattern focussed on the genetic manipulation of genes encoding enzymes of the glycosylation machinery thereby artificially altering properties of the glycosylation pathway in existing production host cell lines (genetic inactivation or deregulation). Antibodies produced with such an approach could show a reduced content or a complete lack of certain glycostructures or linkages. Such genetically modified cell lines can be screened to obtain mutants capable of synthetizing a certain desired glycosylation pattern. The transgenic overexpression of the gene encoding the glycosyl transferase GntIII in CHO cells resulted in cells capable of generating antibody products with an altered glycosylation pattern and ultimately products with reduced fucosylation, activating the ADCC 20-100 fold stronger than antibodies with unmodified glycans (Umana et al., 1999). Yet, it has to be considered, that such genetically modified host cell lines require active selection to stabilize the effect of the genetic alteration, furthermore genetic engineering can have unwanted side effects which increases the regulatory burden for industrial production processes.

The Lectin-resistant CHO mutant cell line Lec13 (Ripka et al., 1986), due to a genetic defect, lacks the enzyme converting GDP-mannose to GDP-fucose. If the cells can not metabolize Fucose from alternative (e.g. external) sources, these cells can be used to produce products with reduced fucosylation and increased activity in the ADCC (Shields et al., 2002). However, the mutation which Lec13 cells contain in their genome, are only maintained upon continuous selection with the Lectin Lens culinaris agglutinin (LCA). Yet, the addition of Lectins or other selection substances in large scale production processes is highly doubtful from a regulatory point of view. Furthermore, protein productivities of Lec13 cells are known to be low, with the fucose content being only partially reduced but highly variable (Kanda et al., 2006; Shields et al., 2002).

### SUMMARY OF INVENTION

The present invention surprisingly demonstrates that the rat hepatoma cell line H4-II-E is a superior production cell line with improved glycosylation properties for highly active biopharmaceutical glycoproteins such as antibodies, especially therapeutic antibodies, and Fc-fusion proteins.

The present invention furthermore describes the analysis of selected non-engineered cell lines representing various species, tissues, cell types and stages of differentiation/tumorigenesis for their applicability as host cell lines for the production of biopharmaceuticals with advantageous glycosylation patterns and thereby improved effector functions.

Surprisingly, the rat hepatoma cell line H4-II-E is the only cell line among several rat and/or liver derived cell lines, combining a number of favourable glycoproperties for an improved efficacy of therapeutic glycoproteins.

The present invention further describes for the first time that H4-II-E rat hepatoma cell lines can be used as a host cell line for the production of recombinant glycoproteins like antibodies or Fc-fusion proteins. The present invention demonstrates that H4-II-E rat hepatoma cells can be transfected stably with genetic elements encoding the light and heavy chain of an antibody or Fc-fusion protein and the derived production cells secrete highly active functional antibody molecules into the cell culture supernatant from where it can be purified. Recombinant antibodies produced in H4-II-E cells, due to the cells glycosylation capacity, are superior in quality and activity to conventionally produced therapeutic antibodies.

The H4-II-E cell line of the present invention has been deposited under the Budapest treaty with the Deutsche Sammlung von Mikroorganismen and Zellkulturen GmbH (DSMZ), Inhoffenstrasse 7B, D-38124 Braunschweig, Germany under the accession number DSM ACC3129 (H4-II-E) on 28 Jun. 2011.

The glycopatten of antibodies, especially IgG1 antibodies, produced in H4-II-E cells shows complex biantennary glycans which are largely fucose-free and at the same time higher galactosylated than antibodies commonly produced in CHO. Thereby, biotherapeutic antibodies produced in H4-II-E cells have a high potential to activate antibody mediated effector functions like the ADCC and CDC vigorously and efficiently.

The present invention furthermore describes that H4-II-E rat hepatoma cell lines can be cultured in suspension and in serum-free media, which is mandatory for their use as production cell lines for biotherapeutics. Surprisingly, after the adaptation of H4-II-E cells to the growth in suspension in chemically defined, animal component free, and calcium ion free medium, the growth, viability and doubling time was not impared compared to the commonly used culture format of H4-II-E cells as adherent cell layers in serum containing media. Doubling times of cultures of H4-II-E cells in serum-containing adherent cultures, as well as after the adaptation to serum-free, animal component free, and calcium ion free medium were in the range of 24 hours to 32 hours or in the range of 32 hours to 60 hours during the adaptation phase. The critical aspect for culturing of H4-II-E cells in suspension with population doubling times of 24 hours to 32 hours is the use of a calcium-free medium. In contrast to this, serum-free cultivation of H4-II-E cells in chemically-defined media was previously described in the literature, yet with population doubling times of 68.5 hours (Miyazaki et al., 1991) or 4 days (Niwa et al., 1980), which is much too slow to fulfil the requirements for a production cell line for biopharmaceuticals.

The H4-II-E cell line adapted to the growth in suspension in serum-free, Ca2+-free medium described in the present invention has been deposited under the Budapest treaty with the Deutsche Sammlung von Mikroorganismen and Zellkulturen GmbH (DSMZ), Inhoffenstrasse 7B, D-38124 Braunschweig, Germany under the accession number DSM ACC3130 (H4-II-Es) on 28 Jun. 2011.

The present invention describes for the first time, a selection of cell lines originating from different species and tissues, which are analysed as related to their suitability for the production of recombinant proteins with advantageous glycosylation patterns (reduced fucosylation, presence of terminal NeuAc-residues). The analysis revealed that only the rat hepatoma cell line H4-II-E is capable of producing several beneficial glycosylation patterns which can significantly improve the activity, efficacy and stability of therapeutic proteins, especially glycoproteins such as antibodies.

H4-II-E cells were originally derived from a hepatocellular carcinoma in the rat (REUBER, 1961) and analysed as an in vivo model for the adapting liver. In contrast to this, the present invention for the first time describes the use of H4-II-E cells as a production system for highly active therapeutic proteins. H4-II-E cells, originating from the rat liver, do not naturally produce antibodies, and H4-II-E cells have not been used for recombinant protein production before. H4-II-E cells originate from a hepatocellular carcinoma in the rat (PITOT et al., 1964; REUBER, 1961) and are used exclusively for toxicological analyses and as a model system to study cellular stress responses (e.g. (Horikoshi et al., 1988; Houser et al., 1992)).

It is very surprising that particularly rat cells, and especially H4-II-E cells, produce glycoproteins or glycosylations with a low content of fucose. In contrast to other species like rabbit or cat, antibodies from normal rat blood serum are heavily fucosylated (Raju et al., 2000). Hamako and colleagues analysed the glyco-composition of serum IgG antibodies from different species and found that 92.8% of the endogenous antibodies from rat blood serum are fucosylated, a higher proportion than in serum IgG of most other species (Hamako et al., 1993).

Thus, it was expected that the rat H4-II-E cells would produce glycoproteins or glycosylations with a high content of fucose rather than with a low content of fucose.

But unexpectedly the opposite was the case for the rat hepatoma cells, the H4-II-E cells. The present invention shows that the H4-II-E cell line (e.g. the cell deposited with the DSMZ under the accession number DSM ACC3129 (H4-II-E) and the cell deposited with the DSMZ under the accession number DSM ACC3130 (H4-II-Es)), derived from rat liver, can surprisingly be distinguished from numerous other cell lines derived from different species by its capacity to produce antibodies with a significantly lower content of fucose than any of the other cell lines.

Shinkawa and colleagues describe the use of the rat myeloma cell line YB2/0 for antibody production (Shinkawa et al., 2003). YB2/0 cells are antibody producing cell lines naturally generating a 34-91% reduced fucosylation of Fc glycostructures, thereby yielding an up to 50fold increased activity in ADCC assays. Thus, YB2/0 cells are in contrast to H4-II-E cells known producer cells. However, being a myeloma cell line, YB2/0 cells are rather sensitive to apoptosis and show a low robustness towards cellular stresses, making this cell line inapplicable for industrial production processes. As reported, the fucosylation in YB2/0 cells furthermore highly variable, resulting in difficulties to control and maintain fucosylation within certain threshold values. In contrast, H4-II-E cells described in the present invention surprisingly show a higher and more consistent degree of defucosylation than reported for YB2/0 cells. Furthermore, the H4-II-E cell is robust and insensitive to stress or other apoptotis inducing stimuli as shown in many toxicological studies in which H4-II-E cells are commonly used as a model system. The direct comparison of the sensitivities of H4-II-E cells and YB2/0 cells to different cellular stresses (osmotic stress, temperature stress, mechanical stress, chemical stress) revealed that H4-II-E cells are superior to YB2/0 cells in every respect. The H4-II-E cell is therefore unexpectedly much better suited for the use as host cell line for industrial production processes than the YB2/O cells.

Another previous approach to obtain products with non-fucosylated glycostructures is the targeted inactivation of the gene encoding the fucosyl transferase 8 (Fut8) in CHO, which results in the complete loss of α1,6-fucosylation in the glycans of secreted proteins like antibody products (Yamane-Ohnuki et al., 2004). An alternative approach applying the siRNA knockdown technology to impair Fut8 gene activity allows the production of partially defucosylated antibodies (Mori et al., 2004). Such antibodies show a 50-100fold stronger activation of the ADCC. Yet, a drawback of these strategies in contrast to the use of H4-II-E cells as production cells is, that the genetic engineering of existing host cell lines certainly affects many more cellular processes than just the glycosylation of the secreted protein of interest. Genetically engineered host cell lines in contrast to H4-II-E cells could, in addition to the desired mutational effect, show changes, like reduced productivity or instability or unpredictable problems, e.g. at later stages in the development of a production process. Furthermore, genetically engineed cell lines often require active selection to stabilize the effect of the genetic alteration, thereby increasing the regulatory burden for industrial production processes.

In addition to showing reduced fucosylation, H4-II-E cells show extra beneficial properties like increased galactosylation and sialylation, which are not found in Fut8 deficient CHO cells.

### ADVANTAGES

The following properties of candidate cell lines are regarded advantageous as related to the existing production cell lines:

Cell lines should be natural (naïve), non genetically engineered host cell lines.
Cell lines should be capable of producing proteins with advantageous glycosylation patterns.
Cell lines should stably show the desired glycopattern without the need for selection (e.g. resistance to lectins).
Such cell lines can be cultured in suspension and serum-free media.
Such cell lines are characterized by a high robustness and low sensitivity to stress or apoptosis stimuli.

The rat hepatoma cell line H4-II-E described in the present invention combines most of these favourable attributes.

In contrast to previous approaches to obtain antibodies with reduced fucosylation, which were principally based on the genetic engineering of existing production cell lines, H4-II-E cells naturally (i.e. without genetic manipulation, mutagenesis or selection) are eligible for the production of recombinant proteins with advantageous glycosylation patterns and their therapeutic use.

In contrast to proteins manufactured in currently used production host cell lines, antibodies produced in H4-II-E show a combination of different beneficial glycosylation properties strongly improving the product quality:
Low fucosylation or lack of fucose: >80% non fucosylated biantennary glycans
   →resulting in an increased potential to activate effector functions like ADCC
High galactosylation: >40% galactosylated biantennary glycans
   →resulting in an increased potential to activate effector functions like CDC

Antibodies produced in H4-II-E cells thereby have a high potential to activate antibody dependent effector functions like ADCC and CDC. This activation is linked to the binding of the antibody to different Fc receptors. Besides the higher activation of the FcgRIIIa, antibodies produced in H4-II-E have the potential to activate the inhibitory receptor FcγRIIb to a lesser extend. This beneficial effect enables the antibody produced in H4-II-E to enhance the immune response mediated by macrophages as especially neutrohpils express also the inhibitory receptor. Therapeutic antibodies produced in H4-II-E cells are therefore superior to conventionally produced antibodies in their therapeutic efficiency particularly towards oncological targets, but also for other indications.
Absence of immunogenic residues: lack of Gal-α1,3-Gal-linkages and NeuGc-residues
   →no allergenic or immunogenic reaction

The lack of potentially immunogenic glycostructures like Gal-α1,3-Gal-linked sugars or NeuGc-residues is another advantage of glycoproteins such as antibodies or Fc-fusion proteins produced in H4-II-E cells. Such structures, which can be found in antibodies which are produced in the mouse myeloma cell lines NS0 or SP2/0 can induce undesired inflammatory reactions or immunogenic rejection, if applied to susceptible patients.
Sialylation: terminal α2,3 or α2,6-linked NeuAc-residues
   →Increased serum stability (serum half life)

Terminal sialylation of the glycans of antibodies produced in H4-II-E cells, has an additional positive effect on the serum stability and catabolic half-life of therapeutic antibodies. H4-II-E rat hepatoma are cells are not genetically engineered and do not need to be selected or cultured in the presence of lectins to maintain their ability to produce beneficial glycosylation patterns.

Furthermore, H4-II-E cells can be cultured in suspension and serum-free media. Even though H4-II-E cells (e.g. the cell deposited with the DSMZ under the accession number DSM ACC3129 (H4-II-E)) are normally cultured adherently using media containing serum (as it is also recommended by the American Type Culture Collection (ATCC, deposit CRL-1548) or the European Collection of Cell Cultures (ECACC, deposit 87031301)), it could be demonstrated in the present invention that H4-II-E cells can be successfully adapted to growth in suspension and serum-free media without impairing the population doubling times of 24-32 hours compared to the growth in adherent cultures in serum-containing media (e.g. the cell deposited with the DSMZ under the accession number DSM ACC3130 (H4-II-Es)).

The doubling time of H4-II-E cells in serum-free media is 24-32 hours. A critical factor to achieve constant growth in suspension and serum-free medium with such favorable doubling times in H4-II-E cells is the omission of calcium in the medium. If calcium is present in the cell culture medium, H4-II-E cells do not grow in suspension and the doubling times are much higher, which makes the application towards biopharmaceutical production unattractive on a commercial scale.

Another advantage of H4-II-E cells for the use as a host cell in biopharmaceutical production processes is their high robustness and low stress or apoptosis sensitivity. Other production cell lines like the myeloma cell line YB2/0 also show beneficial glycopatterns, but at the same time are very sensitive to any kind of stress and therefore not suitable to be cultured in large scale production processes.

In addition to the previously mentioned advantages, therapeutic proteins produced in H4-II-E cells have the following additional beneficial properties:

higher effectiveness and stability=reduced doses required
increased patient convenience (reduced treatment (infusion) frequencies)
reduced risk of side effects through reduced circulating doses
reduced timelines and costs for supply of clinical and market material
→together: Reduced treatment costs

### APPLICABILITY

H4-II-E rat cells can be used for the industrial production of therapeutically highly active proteins, preferably antibodies or Fc-fusion proteins leading to an efficient activation of effector functions after being administered to patients.

Having a significantly reduced content of core fucose, H4-II-E produced antibodies or Fc-fusion proteins show a significantly higher affinity to the polymorphic receptor FcγRIII (CD16-F158, CD16-V158) and activate the inhibitory receptor FcγRIIb to a lesser extent. Thereby, H4-II-E produced antibodies, in contrast to fucosylated CHO produced antibodies, quickly and efficiently recruit and activate not only CD16 positive cells but also macrophages and neutrohpils for the activation of the cytotoxic functions of the immune system. Showing a higher galactosylation than conventionally produced antibodies, products modified and secreted in H4-II-E cells can furthermore bind to components of the complement system (i.e. C1q) more efficiently and thereby activate another cascade which can ultimately lead to the killing of the target cell.

For multiple reasons (see above described advantages), H4-II-E cells are the cell line of choice for the production of antibodies or Fc-fusion proteins, especially those recognizing oncological targets. The improved activation of the effector functions ADCC and CDC by antibodies or Fc-fusion proteins produced in H4-II-E cells, allows an efficient therapy also of solid tumors which are normally not attacked efficiently by the patients immune system after antibody therapy.

The improved serum stability of antibodies produced in H4-II-E cells is beneficial in many therapeutic fields, particularly in chronic diseases, where nowadays, therapeutic substances have to be delivered to patients frequently and repeatedly.

### DESCRIPTION OF THE FIGURES

FIG. 1: Predictive relative content of glycostructures on glycoproteins produced in different cell lines.

Glycoproperties of established cell lines originating from different species and different tissues within these species, are analysed. The relative content of Fucose, α-2,6 sialylated structures, N-Acetlyneuraminic acid (NeuAc), Galactose-1,3-Galactose (Gal-1,3-Gal), and of N-Glycolylneuraminic acid (NeuGc) on proteins produced in these cell lines is estimated by measuring enzymatic activities in the cells and analysing structures on the cell surfaces. The obtained values are normalized to the results obtained in the Chinese hamster ovary cell line (CHO) and are plotted as the predicted relative content of the respective glycostructures. Abbreviations: ha, hamster; mo, mouse; gp, guinea pig; rab, rabbit; go, goat; sh, sheep; hu, human; ch, chicken; du, duck; te, testis; ov, ovary; pa, pancreas; ki, kidney; li, liver; care, hepatoma/carcinoma; eo, esophagus; lu, lung; br.canc, breast carcinoma; co.carc, colon carcinoma, my, myeloma; pr.ly, primary lymphoblastoide cells; pr.b.m, bone marrow stem cells; emb.fib, embryonic fibroblasts.

FIG. 2: IgG1 antibodies produced in CHO, Lec13, YB2/0 and H4-II-E cells differ in their glycosylation pattern: Fucosylation.

The structure and composition of the Fc glycans of IgG1 antibodies produced in CHO-DG44 cells, CHO-Lec13 mutants, YB2/0 rat myeloma cells, and H4-II-E rat hepatoma cells are analysed. The glycans are released from the purified antibody after reduction by enzymatic digestion with PNGase F. Glycans are purified, fluorescently labelled with 2-Aminobenzamide (2-AB) and fractionated on a HPLC column before and after treatment with the exoglycosidic enzyme α-fucosidase or other exoglycosidases. The majority of glycostructures are non-sialylated biantennary glycans. The proportion of fucosylated and non-fucosylated biantennary glycans, and of other glycosidic structures (sialylated glycans, high-mannose structures, or hybrid glycans) are calculated from the chromatographic peak area ratios before and after exoglycosidic digestion.

FIG. 3: Glycopattern of IgG1 antibodies produced in CHO and H4-II-E: Galactosylation

The structure and composition of the Fc glycans of IgG1 antibodies produced in CHO-DG44 cells and H4-II-E rat hepatoma cells are analysed. The glycans are released from the purified antibody after reduction by enzymatic digestion with PNGase F. Glycans are purified, fluorescently labelled with 2-Aminobenzamide (2-AB) and fractionated on a HPLC column before and after treatment with the exoglycosidic enzyme β-galactosidase or other exoglycosidases. The percentages of galactosylated vs. non-galactosylated biantennary glycans are calculated from the chromatographic peak area ratios before and after exoglycosidic digestion.

FIG. 4: Glycopattern of IgG1 antibodies produced in CHO and H4-II-E: Sialylation

The structure and composition of the Fc glycans of IgG1 antibodies produced in CHO-DG44 cells and H4-II-E rat hepatoma cells are analysed. The glycans are released from the purified antibody after reduction by enzymatic digestion with PNGase F. Glycans are purified, fluorescently labelled with 2-Aminobenzamide (2-AB) and fractionated on a HPLC column before and after treatment with the exoglycosidic enzyme Neuraminidase or other exoglycosidases. The percentage of sialylated biantennary glycans is calculated from the chromatographic peak area ratios before and after exoglycosidic digestion.

FIG. 5: Adaptation of H4-II-E cells to growth in suspension in serum-free medium

(A) Adherent growth of H4-II-E cells in MEMalpha medium containing 10% FCS. (B) Suspension culture of H4-II-E cells after adaptation to growth in shaking flasks in serum-free, Ca-free medium. (C) Growth curves of H4-II-E cultures in BI SFM medium seeded at two different inoculum cell densities. Cultures are incubated at 37° C., 5% CO₂ and 120 rpm in shaking flasks. The viable cell concentration is measured at the indicated time points. Abbreviations: BI-SFM, Boehringer Ingelheim Serum Free, Calcium-free Medium; FCS, Fetal Calf Serum; VCC, viable cell concentration; 4*10⁵ cells/ml=400.000 cells/ml; 6*10⁵ cells/ml=600.000 cells/ml.

FIG. 6: Low sensitivity to apoptosis and high robustness of H4-II-E cells towards cellular stresses

(A) Relative viable cell density and viability of H4-II-E cells (black bars) and YB2/0 cells (grey bars) after heating a cell suspensions containing equal cell numbers to 42° C. for 2 hours and subsequent cultivation at 37° C., 5% CO₂ for 22 hours. Control cells are cultured at 37° C., 5% CO₂ for 24 hours. H4-II-E cells show a significantly higher viable cell density and viability after heat stress. (B) Relative viable cell density and viability of H4-II-E cells and YB2/0 cells after 24 hours of cultivation in medium diluted with demineralized water for low ionic strength or 10×PBS for high ionic strength at 37° C., 5% CO₂. Control cells are cultured in undiluted culture medium. H4-II-E cells show a significantly higher viable cell density and viability after cultivation at low or high osmolarity. (C) Relative viable cell density and viability of H4-II-E cells and YB2/0 cells after treatment of cell suspensions containing equal cell numbers with 2 μg/ml or 5 μg/ml Puromycin at 37° C., 5% CO₂ for 48 hours. Control cells are cultured in medium without Puromycin at 37° C., 5% CO₂ for 48 hours. H4-II-E cells show a significantly higher viable cell density and viability after drug treatment.

FIG. 7: Requirement of Ca²⁺-reduced or Ca²⁺-free media for single cell suspension cultivation of H4-II-E cells.

(A) The Ca content of two media suitable for the suspension cultivation of H4-II-E cells is analysed using a Hitachi 917 (Roche). AEM medium containing very low Ca-concentrations and a Ca-free version of a BI proprietary medium, both are suitable for single cell suspension cultivation of H4-II-E cells (B, D). (B)-(E) H4-II-E cells adapted to suspension growth in AEM or in BI (Ca-free) medium, are seeded at a density of 3*10⁵ cells/ml=300.000 cells/ml in the indicated media with or without the addition of CaCl₂ in 12-well-plates. The growth morphology and cell aggregation is analysed microscopically 3 days after seeding. (B) H4-II-E cells grow in single cell suspension in AEM medium. (C) H4-II-E cells form large aggregates in AEM supplemented with 1 mM=1 mMol/L CaCl₂. (D) H4-II-E cells grow in single cell suspension in BI proprietary Ca-free medium. (E) H4-II-E cells form large aggregates in BI medium (containing 1.38 mM=1.38 mMol/L Ca according to the analysis in (A)). (F) H4-II-E cells adapted to suspension growth in BI (Ca-free) medium, are seeded at a density of 3*10⁵ cells/ml=300.000 cells/ml in BI medium (containing Ca) in 12-well-plates and the indicated amounts of EDTA are added to the cultures. The growth morphology and cell aggregation is analysed microscopically 3 days after seeding. Note that the dose dependent, EDTA mediated depletion of free Ca²⁺ from the medium decreases the cell aggregation and increases the proportion of suspended single cells.

FIG. 8: Ca²⁺-concentration dependent, and Mg²⁺-independent aggregation of suspended H4-II-E cells.

(A) H4-II-E cells adapted to suspension growth in BI (Ca-free) medium, are seeded at a density of 4*10⁵ cells/ml=400.000 cells/ml in BI (Ca-free) medium in 12-well-plates and the indicated amounts of CaCl₂ are added to the cultures. The growth morphology and cell aggregation is analysed microscopically 2 days after seeding. Note the concentration dependent, Ca²⁺ mediated cell aggregation of suspended H4-II-E cells. (B) H4-II-E cells adapted to suspension growth in BI (Ca-free) medium, are seeded at a density of 4*10⁵ cells/ml=400.000 cells/ml in BI (Ca-free) medium in 12-well-plates and the indicated amounts of MgCl₂ are added to the cultures. The growth morphology and cell aggregation is analysed microscopically 2 days after seeding. Note that cells grow in single cell suspension independent of the Mg²⁺ concentration. (C) H4-II-E cells adapted to suspension growth in AEM medium, are seeded at a density of 3*10⁵ cells/ml=300.000 cells/ml in AEM medium in shake flasks and the indicated amounts of CaCl₂ or of MgCl₂ are added to the cultures. The cultures are incubated at 37° C., 5% CO₂, and 120 rpm. The viable cell density and cell aggregation rate is analysed with the CEDEX cell quantification system (Innovatis). Note that the viable cell density of the cultures significantly drops if the CaCl₂ concentration in the medium is higher than 100 μM. In contrast, increasing concentrations of MgCl₂ have no effect on the viable cell density. Furthermore, note that the cell aggregation rate increases with increasing CaCl₂ concentrations and saturates at CaCl₂ concentrations>250 μM. In contrast, increasing concentrations of MgCl₂ do not have an effect on the aggregation rate of H4-II-E cells.

FIG. 9: Binding affinities of IgG1 produced in CHO and H4-II-E: FcγRIIIa.

The binding kinetics of IgG1 produced in different cell lines to FcγRIIIa is measured using a BIAcore T100 instrument and CM5 sensor chips (BIACORE, Uppsala, Sweden) as follows. Soluble recombinant FcγRIIIa is immobilized onto the BIAcore sensor chip. The purified IgG1s are diluted in HBS-EP buffer (0.01 M HEPES, 0.15 M NaCl, 3 mM EDTA, 0.005% Surfactant P20, pH 7.4) at six different concentrations (from 4.17 to 133.3 nM) and each diluted IgG1 is injected over the receptor-captured sensor surface at a flow rate of 5 mL/min The experiments are performed at 25° C. with HBS-EP as the running buffer. Buffer solution without sample IgG1 is injected over the receptor-captured sensor surface as a blank control. Soluble FcγRIIIa and IgG1 bound to the sensor surface are removed by injecting 7.5 mM HCl at a flow rate of 10 mL/min for 30 s. The data obtained by the injection of IgG1 are corrected for the blank control prior to data analysis. An affinity (KD) for FcγRIIIa is calculated by steady-state analysis using BIAcore T100 kinetic evaluation software (BIACORE). Note that the IgG1 produced in the H4-II-E cell line has a higher binding affinity to the receptor compared to the IgG1 produced in CHO.

FIG. 10: Effector functions of IgG1 produced in CHO and H4-II-E: ADCC.

ADCC assays are performed by the lactate dehydrogenase (LDH) release assay using as effector cells human peripheral blood mononuclear cells (PBMC) prepared from healthy donors by Lymphoprep (AXIS SHIELD, Dundee, UK). Aliquots of target tumor cells, the human Burkitt's lymphoma cell line Ramos, expressing human CD20, is distributed into 96-well U-bottomed plates (10.000 cells in 50 μl/well) and incubated with serial dilutions of antibodies (50 μL) in the presence of the PBMC (100 μL) at an E/T ratio of 20/1. After incubation for 4 h at 37° C., the supernatant LDH activity is measured using a Non-Radioactive Cytotoxicity Assay Kit (Promega, Madison, Wis.). The percent specific cytolysis calculated from the sample activities according to the formula: specific lysis=100*(E−S_{E}−S_{T})/(M−S_{T}). FU stands for fluorescence units.

Note that the IgG1 produced in H4-II-E has a stronger ADCC activation compared to the IgG1 produced in CHO.

FIG. 11: Binding affinities of IgG1 produced in CHO and H4-II-E: FcRn.

The kinetics of the human IgG1-FcRn interaction is measured using a BIAcore T100 instrument and CM5 sensor chips. Antihuman b2-microglobulin monoclonal antibody (Abcam, Cambridge, UK) is immobilized onto the chip using an amine-coupling kit (BIACORE). Soluble FcRn-b2 microglobulin complex is captured by the immobilized anti-b2-microglobulin antibody by injecting the complex at a flow rate of 5 mL/min. Buffer solution without the complex is injected over the antibody-captured sensor surface as a blank control. Each purified IgG is diluted in HBS-EP+ buffer (0.01 M HEPES, 0.15 M NaCl, 3 mM EDTA, 0.05% Surfactant P20) whose pH is adjusted to 6.0 at five different concentrations (from 4.17 to 66.7 nM), and each diluted IgG1 is injected over the complex-captured sensor surface or blank at a flow rate of 5 mL/min Soluble FcRn and IgG1 bound to the sensor surface are removed by injecting 7.5 mM HCl at a flow rate of 60 mL/min for 1 min. The experiments are performed at 25° C. with HBS-EP+ as a running buffer. The data obtained by blank subtraction are used for the data analysis. An apparent association rate constant (ka), a dissociation rate constant (kd), and the binding affinity (KD) are calculated by the bivalent fitting model using BIAcore T100 evaluation software. Note that the antibody produced in H4-II-E binds the FcRn comparably to the antibody produced in CHO.

### LEGEND TO SEQUENCE LISTING

SEQ ID NO 1: amino acid sequence of O-linked glycosylation site
SEQ ID NO 2: amino acid sequence of anti-CD20 IgG1 mAb heavy chain
SEQ ID NO 3: amino acid sequence of anti-CD20 IgG1 mAb light chain
SEQ ID NO 4: amino acid sequence of anti-CD20 IgG4 mAb heavy chain
SEQ ID NO 5: amino acid sequence of anti-CD20 IgG4 mAb light chain
SEQ ID NO 6: amino acid sequence of MCP1-Fc fusion protein
SEQ ID NO 7: amino acid sequence of EPO-Fc fusion protein

### DETAILED DESCRIPTION OF THE INVENTION

Post-translational protein modifications are known to have a crucial effect on the biological activity, stability and immunogenicity of proteins. The most common post-translational modification of secreted proteins is the glycosylation and the majority of all approved therapeutic biopharmaceuticals including recombinant antibodies or recombinant Fc-fusion proteins are glycoproteins.

Since the correct glycosylation pattern is indispensable for the therapeutic activity and specificity, most glycoprotein therapeutics are produced in eukaryotic expression systems capable of generating a complex spectrum of glycosylation patterns. Today, Chinese hamster ovary (CHO) cells have become the standard mammalian host cell used in the production of recombinant proteins. Yet, it is known, that different eukaryotic production cell lines produce distinct Fc glycosylation patterns and thereby have a significant impact on the biological activities of therapeutic antibodies. Furthermore it is known, that certain defined glycostructures are more potent than others in activating particular downstream effector mechanisms. Therapeutic antibodies carrying the most human-like glycosylation patterns, are not particularly efficient in the activation of anti-tumoral effector mechanisms, but, at the same time, such antibodies are rather unlikely in causing unwanted allergic or immunogenic rejection reactions in patients and are therefore regarded as safe. To improve e.g. the cell-killing activities induced by anti-cancer antibodies it is desirable to produce distinct glycosylation patterns. Similarly, an altered glycosylation pattern can improve other antibody dependent effector functions or it can alter the serum-half life, without impairing the safety or stability of the therapeutic product itself.

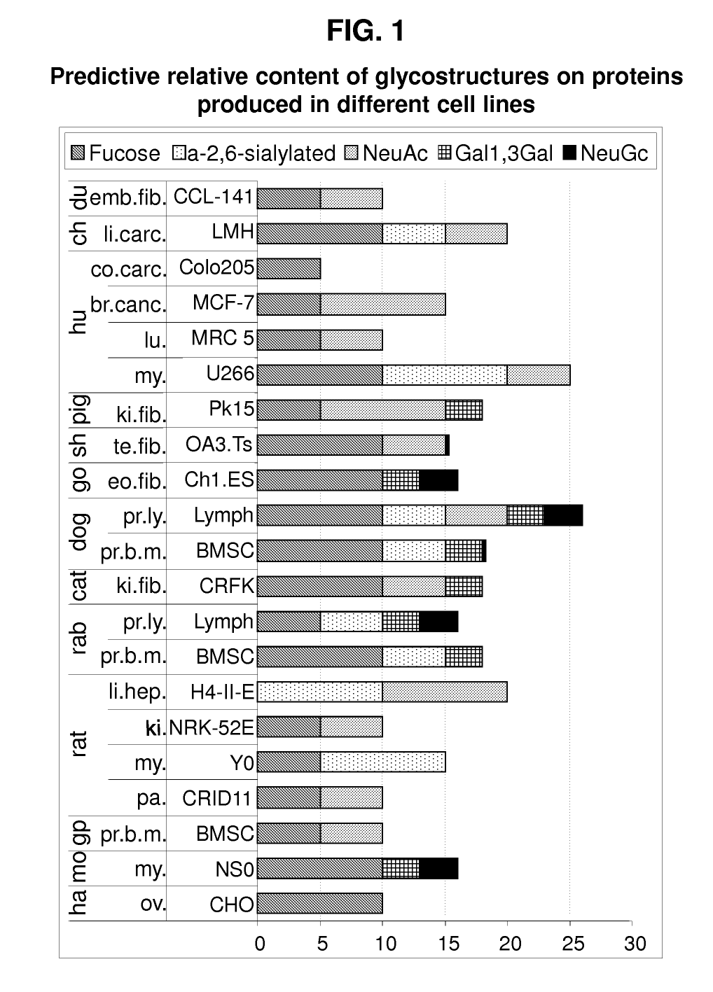

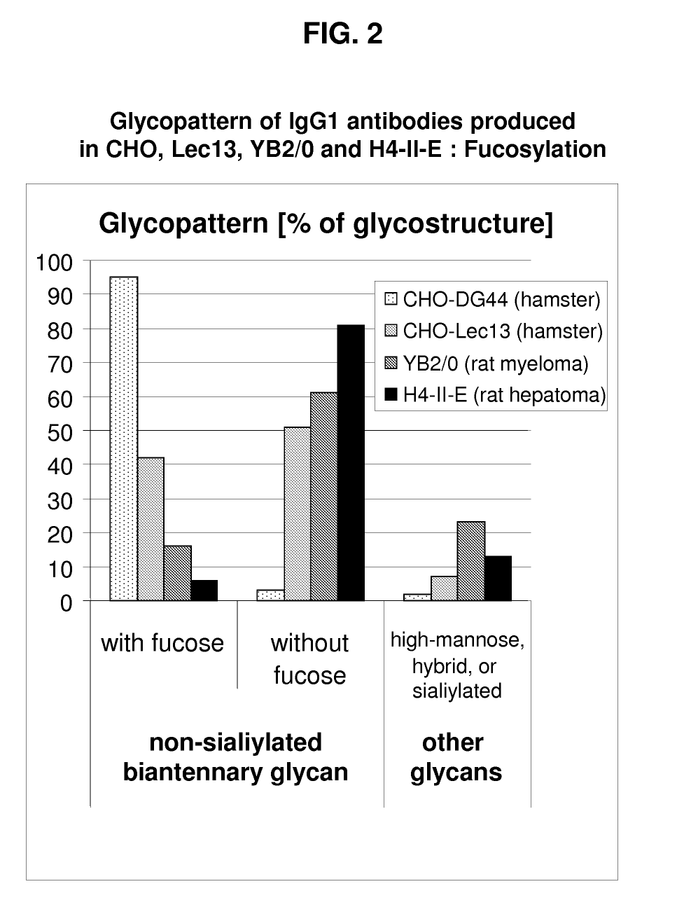

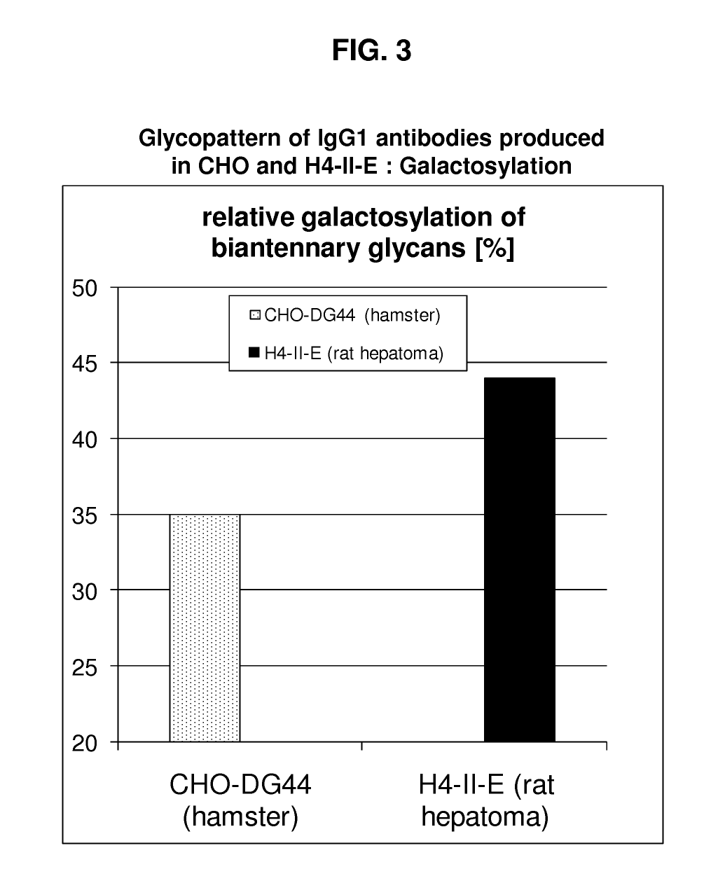

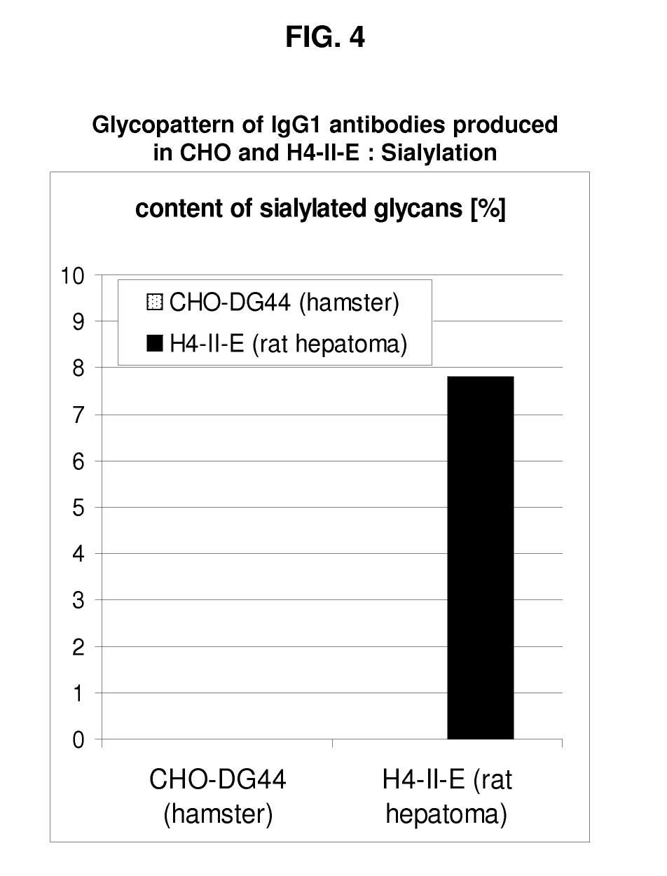

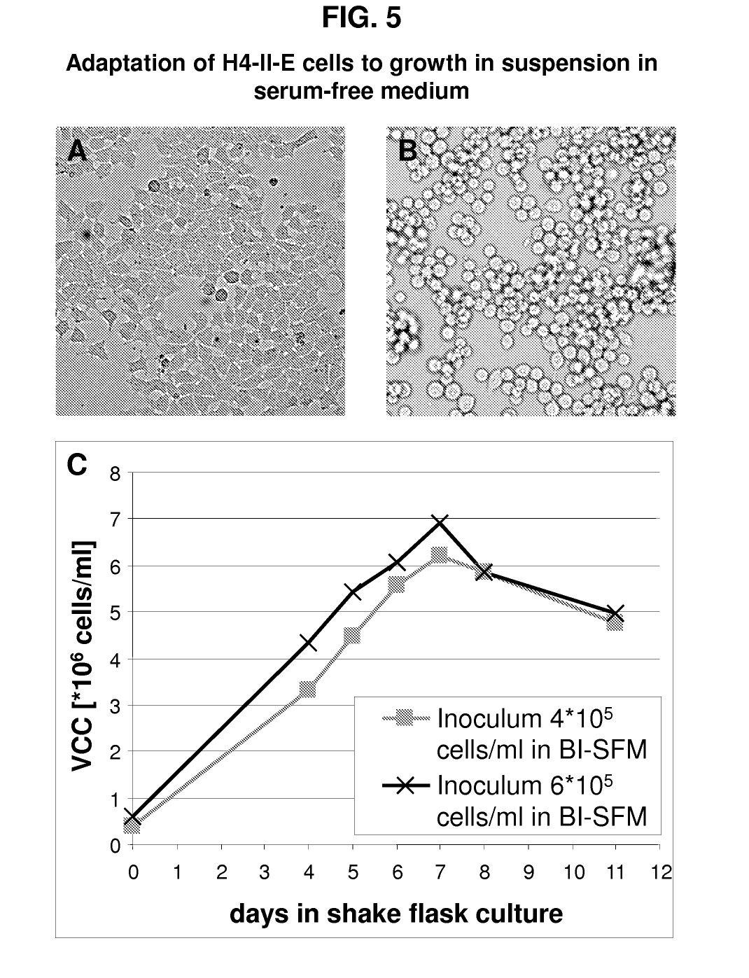

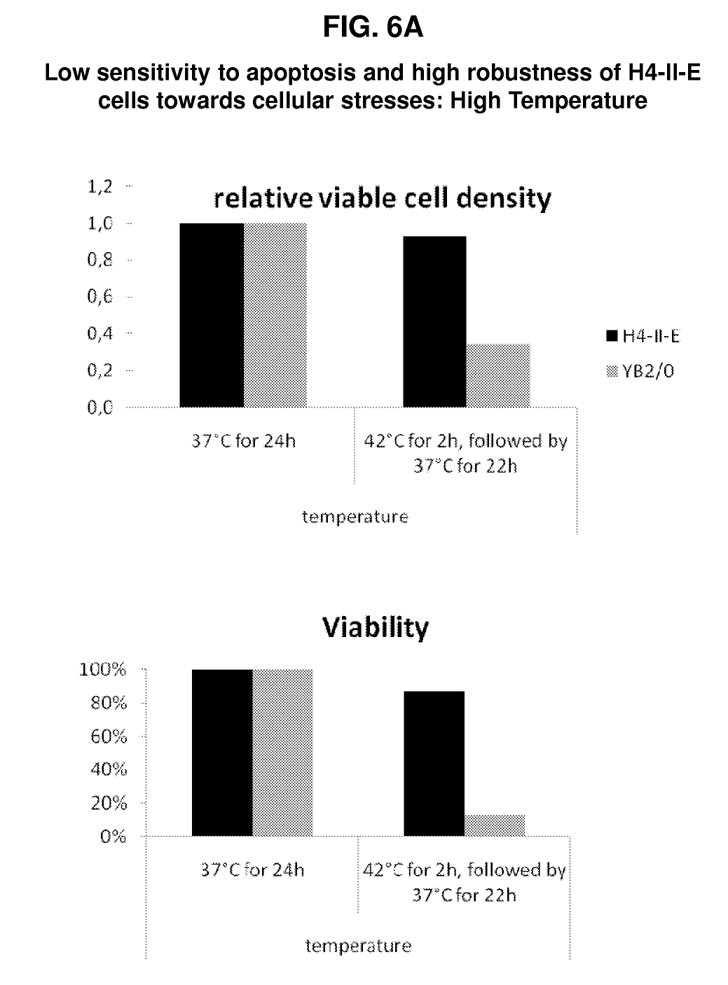

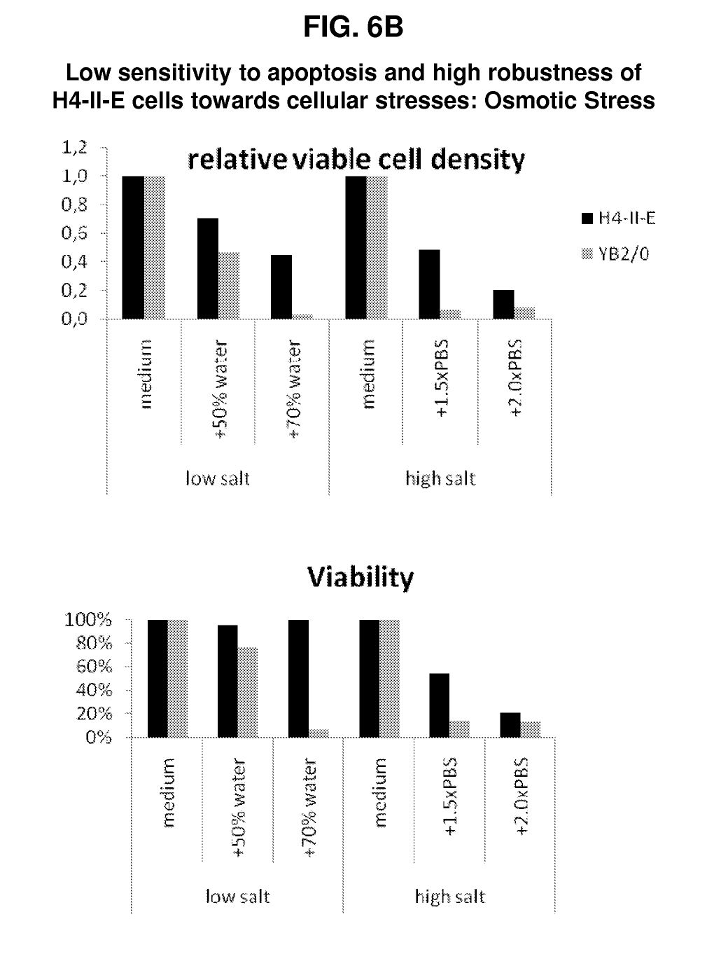

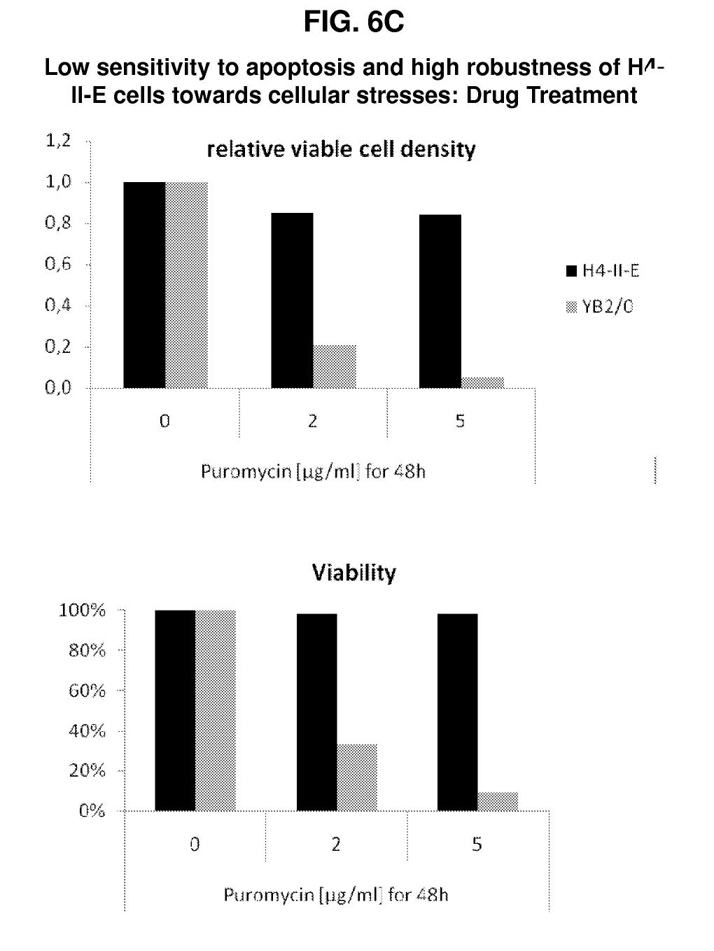

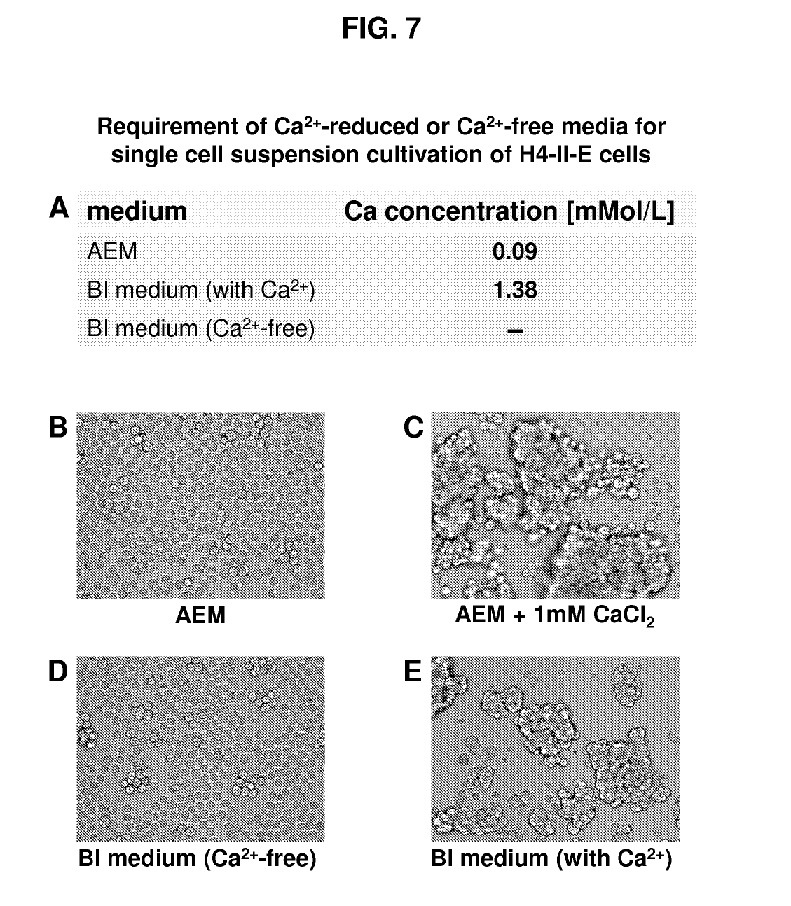

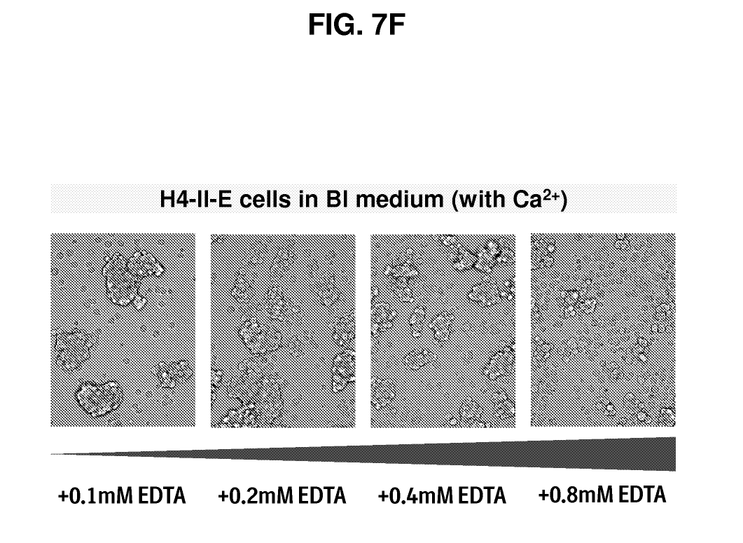

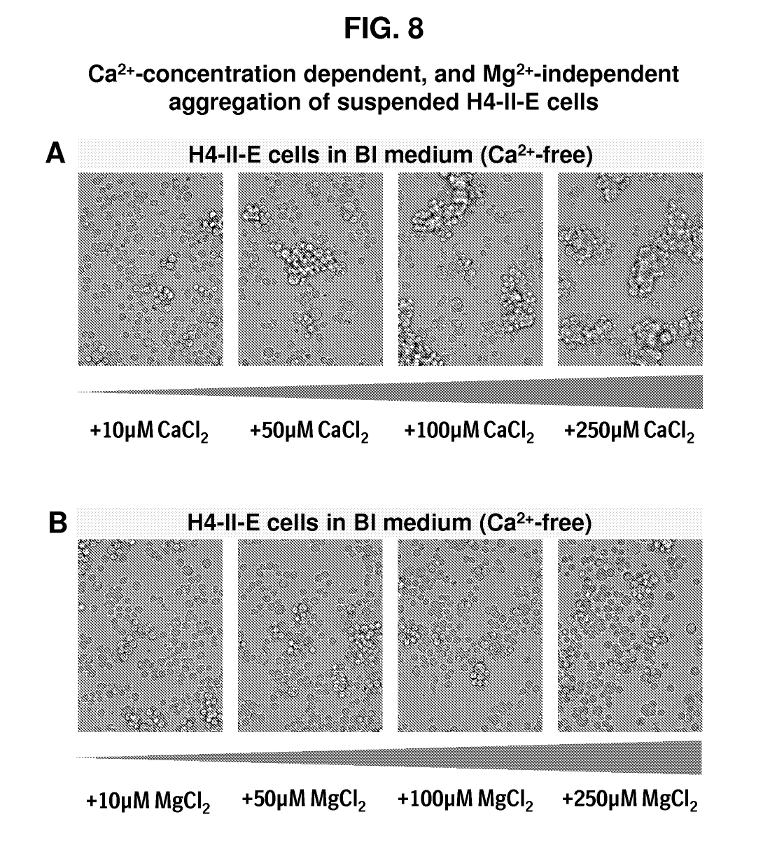

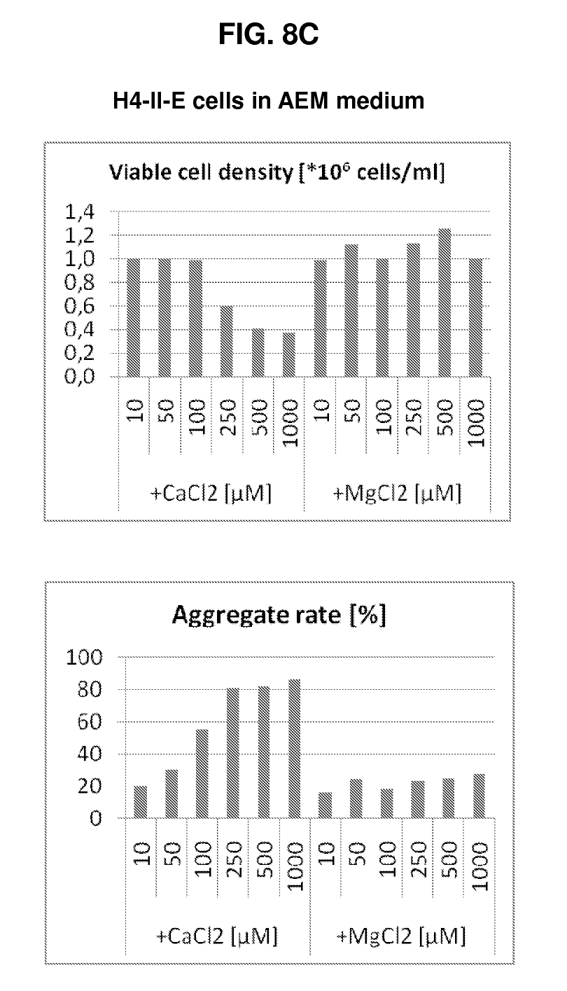

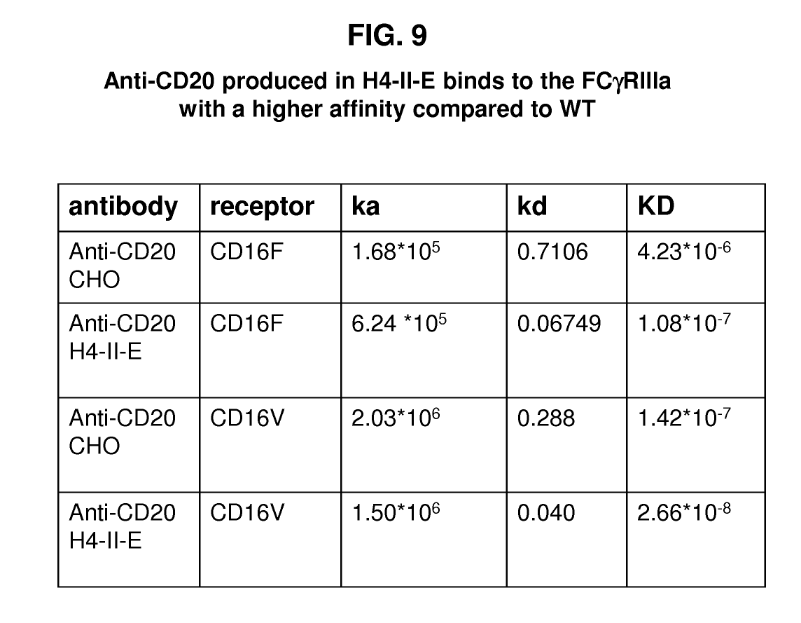

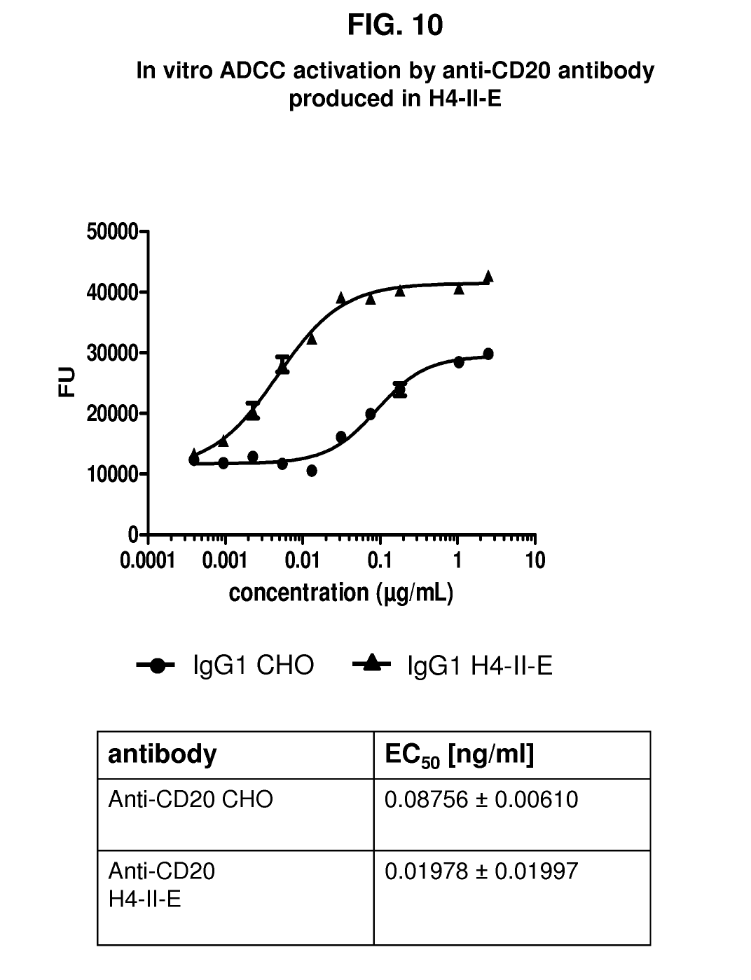

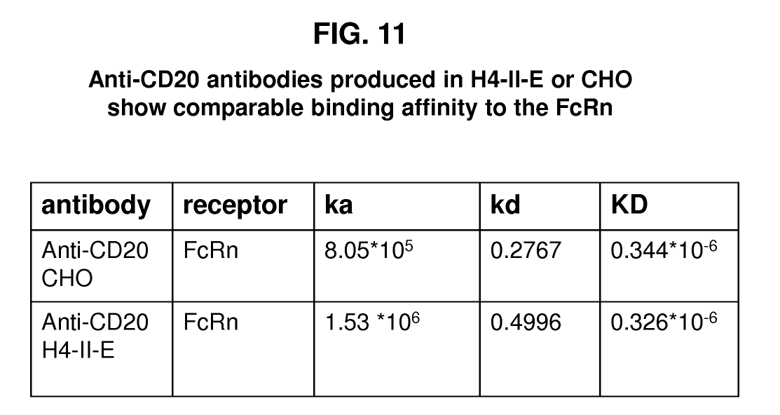

In the present invention we specifically select and analyse different cell lines originating from diverse species, tissues and cell lineages. Glycans exposed on the cell surface as well as enzymatic activities within the cells are examined (FIG. 1). Thereby, each cell line can be assigned the capability of synthesizing certain glycosylation patterns. The selection and analysis shows that cell lines clearly display differences in their glycosylation capacities depending on both, the species and tissue or cellular lineage from which they originate. Only few cell lines are naturally displaying the capability for low fucosylation, a glycosylation trait having a positive impact on the activation of the antibody-dependent cellular cytotoxicity pathway (ADCC). Some cell lines are capable of generating sialylated structures which can improve the serum-stability of therapeutic proteins. Some cell lines produce immunogenic glycostructures (like Gal-1,3-Gal and NeuGc), causing inflammatory reactions in humans. The analysis of the present invention reveals that neither the species origin of a given cell line, nor the tissue, organ or cell lineage alone determine the glycosylation capacity of a cell. Thus, genetic and epigenetic factors influence the ability of a cell to synthesize certain glycosylation patterns. The data of the present invention therefore demonstrate that it is not possible to predict the glycosylation properties of a cell line solely based on the knowledge of the glycopattern derived in another cell line originating from the same tissue and/or species (FIG. 1).

The only cell line for which several properties, like reduced fucosylation, lack of immunogenic residues, and the presence of α-2,6 linked sialic acids are shown, is the rat hepatoma cell line H4-II-E. These properties positively impact the activity and stability of recombinant biotherapeutics particularly of antibodies or Fc-fusion proteins. The selected cell line H4-II-E is the only one of all analysed cell lines, which shows no detectable signs of fucosylation in this analysis. This is surprising, since other rat cell lines and other liver cell lines have different properties. Moreover, rat blood serum antibodies are described to be heavily fucosylated, while other species like rabbit or cat according to the literature have a lower content of fucose on serum antibodies than rat (Raju et al., 2000).

Glycostructures like Gal-1,3-Gal and NeuGc are potentially immunogenic. The H4-II-E rat cell does not show such potentially immunogenic glycostructures. The H4-II-E rat cell furthermore does not produce antibodies or Fc-fusion proteins having such potentially immunogenic glycostructures. Actually, in none of the selected human or rat cell lines such glycostructures like Gal-1,3-Gal and NeuGc, which are potentially immunogenic, showed up, while cell lines derived from mouse, rabbit and other species, consistently produced such structures, which can induce immunogenic reactions in humans. Thus, according to the experimental data of the present invention, these potentially immunogenic glycostructures are attached to secreted glycoproteins by cells in a species-dependent manner.

To verify that H4-II-E cells are capable of producing secreted glycoproteins with the predicted glycosylation properties, stable recombinant antibody producing H4-II-E cells are generated by transfection of corresponding DNA constructs and subsequent selection for cells having stably integrated in the product gene and an antibiotic resistance marker. Antibody producing cell populations are obtained and cultured as adherent cell layers. H4-II-E cells are furthermore adapted to growth in suspension and can be cultured in serum-free, chemically defined medium (FIG. 5). Antibodies are purified from the cell culture supernatants by Protein A chromatography. The Fc glycosylation is analysed and compared to the patterns obtained on antibodies produced in CHO-DG44, CHO-Lec13 mutants and YB2/0 rat myeloma cells. Biantennary glycans make up the largest proportion in all four IgG1 preparations. Within the fraction of biantennary glycans, CHO-DG44 cells, as previously reported, produce largely (˜95%) the fucosylated forms. In contrast, more than 80% of IgG1 expressed in H4-II-E cells contain fucose-free biantennary glycans, which is a significantly higher proportion than if antibodies are produced in the cell lines YB2/0 or the CHO mutant Lec13 (FIG. 2). The detailed analysis of the glycosylation pattern on antibodies produced in H4-II-E cells also reveals that >40% of the glycans are galactosylated, a higher proportion than obtained when antibodies are produced in CHO-DG44, the host cell line which is typically used for biopharmaceutical protein production (FIG. 3). Furthermore, approximately 8% of the antibodies produced in H4-II-E cells carry terminal sialic acid residues which are not normally found if recombinant antibodies are produced in CHO-DG44 cells (FIG. 4). Terminal sialic acid residues can have different effects on the activity and stability of the modified antibodies. Recent publications indicate that sialic acid can inhibit inflammatory activities of antibodies (Burton and Dwek, 2006; Scallon et al., 2007). Other data indicate that the absence of sialic acid leads to an enhanced metabolic rate in the liver of mice, indicating a clearance by liver based receptors (Wright et al., 2000). Derivatives of sialic acid like N-glycolylneuraminic acid (NeuGc) are found to be immunogenic in humans (Noguchi et al., 1995). However, evidence of NeuGc modifications on proteins produced in H4-II-E cells could not be detected (FIG. 1). In summary, H4-II-E derived antibodies lack potentially immunogenic residues.

Altogether, these expression data confirm the results obtained in the predictive analysis for advantageous glycosylation patterns (FIG. 1). In summary, the rat hepatoma cell line H4-II-E distinguishes itself from other cell lines due to its ability to generate several beneficial glycosylation patterns. The described glycosylation patterns obtained after antibody production in H4-II-E cells translate into improved affinity of binding of the antibodies to FcγRIII receptors and superior activity in ADCC assays, stronger binding of components of the complement system and enhances activity in CDC assays, stronger binding to the neonatal

Fc receptor FcRn having a positive effect on the antibody stability and serum half life. In addition to bringing about beneficial glycoproperties, H4-II-E cells are characterized by a high robustness and low sensitivity to stress or apoptosis inducing stimuli. In line with this, H4-II-E cells are adaptable to the growth in suspension and in serum free medium. H4-II-E cells grow well in different cultivation formats and can be cultured with high viabilities in suspension in FedBatch processes for more than 10 days. Taken together, H4-II-E cells bring along excellent qualities for the large scale industrial production of biotherapeutics with outstanding effector activities and increased serum half life.

### DEFINITIONS

The general embodiments “comprising” or “comprised” encompass the more specific embodiment “consisting of”. Furthermore, singular and plural forms are not used in a limiting way. Terms used in the course of this present invention have the following meaning.

The present invention relates to all rat hepatoma cell lines derived from a “Reuber H-35 hepatoma” ((REUBER, 1961) and 1964 (PITOT et al., 1964)). “Reuber H-35 hepatomas” are induced by chemical carcinogenesis in an AxC rat. Cell lines derived from such “Reuber H-35 hepatomas” include for example the H4-II-E and the H4-II-E-C3 cell lines and derivatives or progenies thereof.

More specifically, a “H4-II-E cell” means a cell derived from the European Collection of Cell Cultures (ECACC, Cat. no. 87031301) or from the American Type Culture Collection (ATCC, deposit no. CRL-1548) or originating from the rat hepatoma cell line isolated and firstly described in the literature in 1961 (REUBER, 1961) and 1964 (PITOT et al., 1964). A H4-II-E cell specifically is a cell having the ECACC Cat. no 87031301 or ATCC no. CRL-1548.

The term “H4-II-E cell” furthermore means the H4-II-E-C3 cell line (CRL-1600 or HPACC No. 85061112), the H4II cell line (HPACC Nr. 89042702), which is also derived from the “Reuber H35 hepatoma” and which is the same as H4-11-E-C3 (ECACC catalogue no. 85061112), the H4-TG cell line (CRL-1578), which is a derivative of CRL-1600 having HPRT-deficiency and constant expression of the enzyms phenylalanin hydroxylase, the H5 cell line (HPACC, Nr. 94101905), which is also a subclon of H4-II-E-C3 (CRL-1600), and the H4-S cell line (HPACC Nr. 89102001), which is a rat hepatoma cell infected with VS-virus.

The term “H4-II-E cell” comprises also cells derived from the originally deposited H4-II-E cell and cells, which are progenies of the originally deposited H4-II-E cell or which are derived from the originally deposited H4-II-E cell.

Thus, the term “H4-II-E cell” comprises unmodified or modified descendants/forms of H4-II-E cells. Such unmodified or modified descendants/forms of H4-II-E cells are also referred to as “derivatives or progenies” of the originally deposited H4-II-E cell.

Unmodified forms of H4-II-E cells are all cells created, which constitute an unmodified, functional subunit of H4-II-E cells. Some examples include: subclones of unmodified cell lines, purified or fractionated subsets of it.

Modified forms (or derivatives or progenies) of H4-II-E cells are all cells generated from a parental H4-II-E cell through the introduction of functional DNA sequences, especially those conferring the potential of producing recombinant proteins, particularly glycoproteins including antibodies or Fc-fusion proteins to the respective starting cells.

Modified forms (or derivatives or progenies) of H4-II-E cells are all cells generated from a parental H4-II-E cell through mutagenesis or targeted gene modification or gene integration. Examples of such modified H4-II-E cells are genetically engineered H4-II-E cells comprising a transgene such as lipid transfer protein CERT (also known as Goodpasture antigen-binding protein), a transcription factor such as upstream binding factor UBF or a gene encoding a member of the Sec-1/Munc18 protein family.

Further examples of modified H4-II-E cells are H4-II-E cells, in which a gene or genes such as e.g. DHFR (dihydrofolate dehydrogenase), GS (glutamine synthetase), TIP-5 or SNF 2H are knocked out or knocked down. Especially DHFR or GS knock out H4-II-E cells are useful for biopharmaceutical production due to advantageous selection options in context with recombinant protein expression.

Such DHFR or GS knock out or knock down H4-II-E cells are examples of auxotroph cells. The DHFR knock out or knock down H4-II-E cell is auxotroph for hypoxanthine (H) and thymidine (T). The GS knock out or knock down H4-II-E cell is auxotroph for glutamine. But there are further auxotrophe H4-II-E cells the person skilled in the art can generate. Modified forms (or derivatives or progenies) of H4-II-E cells furthermore means any cell generated from a parental H4-II-E cell through the adaptation to special media, growth, culture format or selection substances.

The term H4-II-E cell specifically relates to two deposited cell lines. The H4-II-E cell line of the present invention has been deposited under the Budapest treaty with the Deutsche Sammlung von Mikroorganismen und Zellkulturen GmbH (DSMZ), Inhoffenstrasse 7B, D-38124 Braunschweig, Germany under the accession number DSM ACC3129 (H4-II-E) on 28 Jun. 2011.

The H4-II-E cell line adapted to the growth in suspension in serum-free, Ca2+-free medium described in the present invention has been deposited under the Budapest treaty with the Deutsche Sammlung von Mikroorganismen und Zellkulturen GmbH (DSMZ), Inhoffenstrasse 7B, D-38124 Braunschweig, Germany under the accession number DSM ACC3130 (H4-II-Es) on 28 Jun. 2011.

The terms “cell” and “cell line” as used in this invention refer especially to expression cells/expression cell lines and host cells/host cell lines.

The term calcium-reduced or preferably calcium-free medium means media that are defined to contain 1 μmol/L-500 μmol/L of Ca2+ ions, more preferred Calcium-reduced media contain 1 μmol/L-250 μmol/L of Ca2+ ions, and even more preferred, Calcium-reduced or Ca-free media contain 0 μmol/L-100 μmol/L or 0.5 μmol/L-100 μmol/L of Ca2+ ions. In AEM medium, the addition of 250 μM/L CaCl₂ or of higher CaCl₂ concentrations significantly impairs the growth of H4-II-E cells already after 3 days in culture. In AEM medium supplemented with 250 μM/L CaCl₂ or of higher CaCl₂ concentrations, more than 80% of the cells are forming compact aggregates. Calcium-reduced media can also be obtained through the addition of EDTA to media originally containing Calcium, thereby reducing the concentration of free Calcium ions in the medium by complex formation with EDTA. EDTA is preferably added in a concentration between 400 μmol/L-1200 μmol/L or 600 μmol/L-1000 μmol/L or 700 μmol/L-900 μmol/L. EDTA is preferably added in a concentration of around 800 μmol/L.

Examples of commercially available Calcium-free media are MEM Joklik Modification (Sigma) or MEM Spinner Modification (Sigma). A typical Calcium-containing medium used for the adherent cultivation of H4-II-E cells in the presence of serum is Eagle's Minimum Essential Medium (Sigma) which contains 1360 μmol/L of Ca2+ ions. A serum-free, calcium-reduced medium used for suspension cultivation of H4-II-E cells is AEM (Invitrogen).

In contrast to calcium ions, magnesium ions do not have an effect on the aggregation rate of rat hepatoma cells/H4-II-E cells. Thus, rat hepatoma cell/H4-II-E cell aggregation is magnesium (Mg2+) ion independent, but calcium (Ca2+) ion dependent.

“Glycosylation sites” refer to amino acid residues which are recognized by a eukaryotic cell as locations for the attachment of sugar residues. The term “glycosidic structure” or “glycan” refers to the sugar residues attached to a glycosylation site. The amino acids where carbohydrates, such as oligosaccharides, are attached are typically asparagine (N-linkage), serine (O-linkage) and threonine (O-linkage) residues. The specific site of attachment is typically defined by a sequence of amino acids, referred to herein as a “glycosylation site sequence”.

The glycosylation site sequence for N-linked glycosylation is -Asn-X-Ser- or -Asn-X-Thr-, where X may be any of the conventional amino acids, other than proline. The predominant glycosylation site for O-linked glycosylation is -(Thr or Ser)-X-X-Pro- (SEQ ID NO 1), where X is any conventional amino acid. The term “N-linked” and “O-linked” refer to the chemical group that serves as the attachment site between the sugar molecule and the amino acid residue. N-linked sugars are attached through an amino group; O-linked sugars are attached through a hydroxyl group. However, not all glycosylation site sequences in a protein are necessarily glycosylated. Some proteins are secreted in both glycosylated and nonglycosylated forms, while others are fully glycosylated at one glycosylation site sequence but contain another glycosylation site sequence that is not glycosylated. Therefore, not all glycosylation site sequences that are present in a polypeptide are necessarily glycosylation sites where sugars are actually attached. The initial N-glycosylation during biosynthesis inserts the “core carbohydrate” or “core oligosaccharide”.

In the N-linked glycosylation the carbohydrate moiety is attached via GlcNAc to an asparagine residue in a polypeptide chain. The N-linked carbohydrates have various structures, but all contain a common core structure in which the terminal GlcNAc which binds to asparagines is called the reducing end and the opposite side is called the non-reducing end:

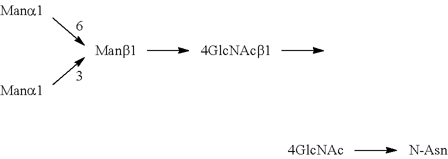

The skilled person will recognize that, for example, each of murine IgG3, IgG1, IgG2B, IgG2A and human IgD, IgG3, IgG1, IgA1, IgG2 and IgG4 CH2 domains have a single, conserved site for N-linked glycosylation at amino acid residue 297 according to the Kabat EU nomenclature (Kabat et al., 1991). The residues in antibody domains are conventionally numbered according to a system set forth by Kabat (Kabat et al., 1991), which refers to the numbering of the EU antibody (Edelman et al., 1969). It should be noted that the Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component. The correct Kabat numbering of residues may be determined for a given antibody by alignment of residues of homology in the sequence of the antibody with a “standard” Kabat numbered sequence. The person skilled in the art will appreciate that these conventions consist of nonsequential numbering in specific regions of an immunoglobulin sequence, enabling a normalized reference to conserved positions in immunoglobulin families.

Of the N-linked carbohydrates the most important are the “complex” N-linked carbohydrates. According to the present invention such complex carbohydrates will be of the “biantennary” structures described herein. The core biantennary structure is typical of biantennary oligosaccharides and can be represented schematically as follows.

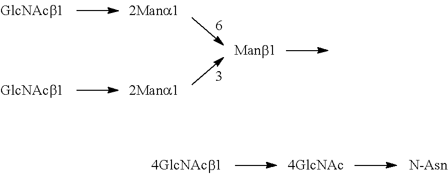

Since each biantennary structure may have a bisecting N-acetylglucosamine (GlcNAc), outer galactose and sialic acid saccharides added to one or both branches at the non-reducing terminal side, and fucose added to the GlcNAc at the reducing end of the core, there are a total of 36 structurally unique complex type oligosaccharides which may occupy the N-linked Asn 297 site.

It will be also recognized that within a particular CH2 domain, glycosylation at Asn 297 may be asymmetric owing to different oligosaccharide chains attached at either Asn 297 residue within the two chain Fc domain (or post-translational trimming). For example, while the heavy chain synthesized within a single antibody-secreting cell may be homogeneous in its amino acid sequence, it is generally differentially glycosylated resulting in a large number of structurally unique immunoglobulin glycoforms (with different biological activity and biophysical properties). The major types of complex oligosaccharide structures found in the CH2 domain of the IgG are shown below.

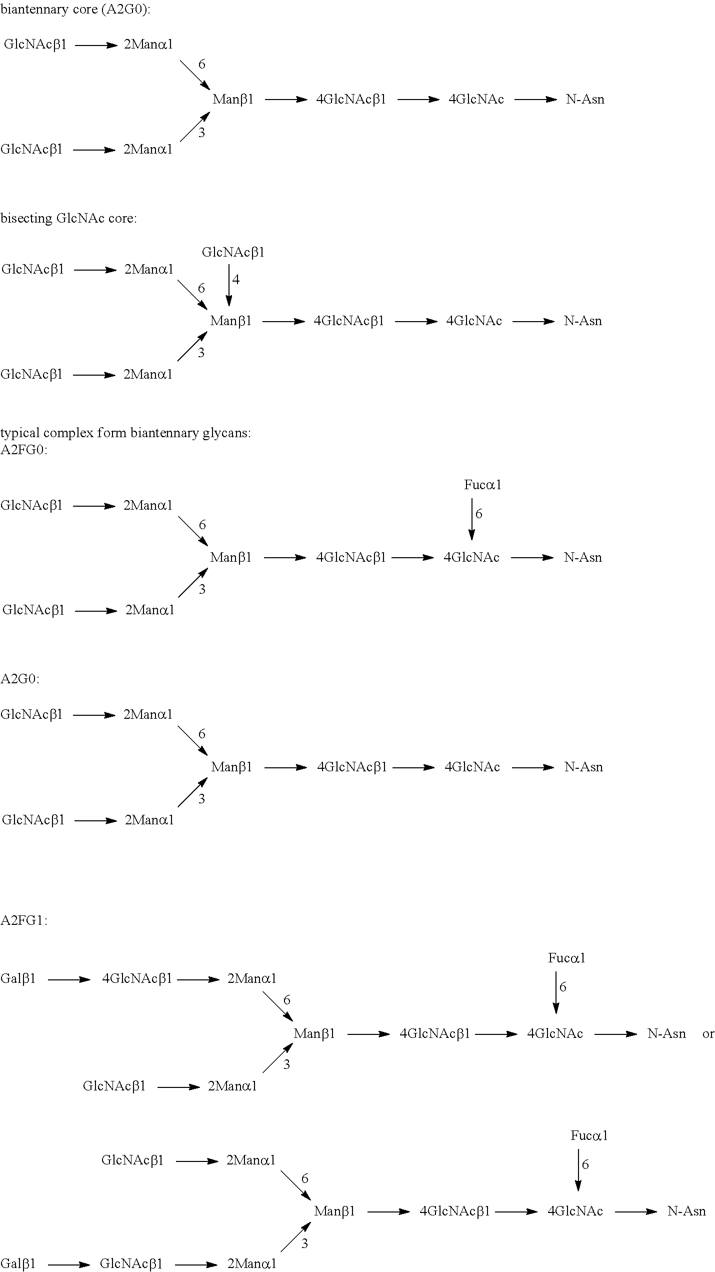

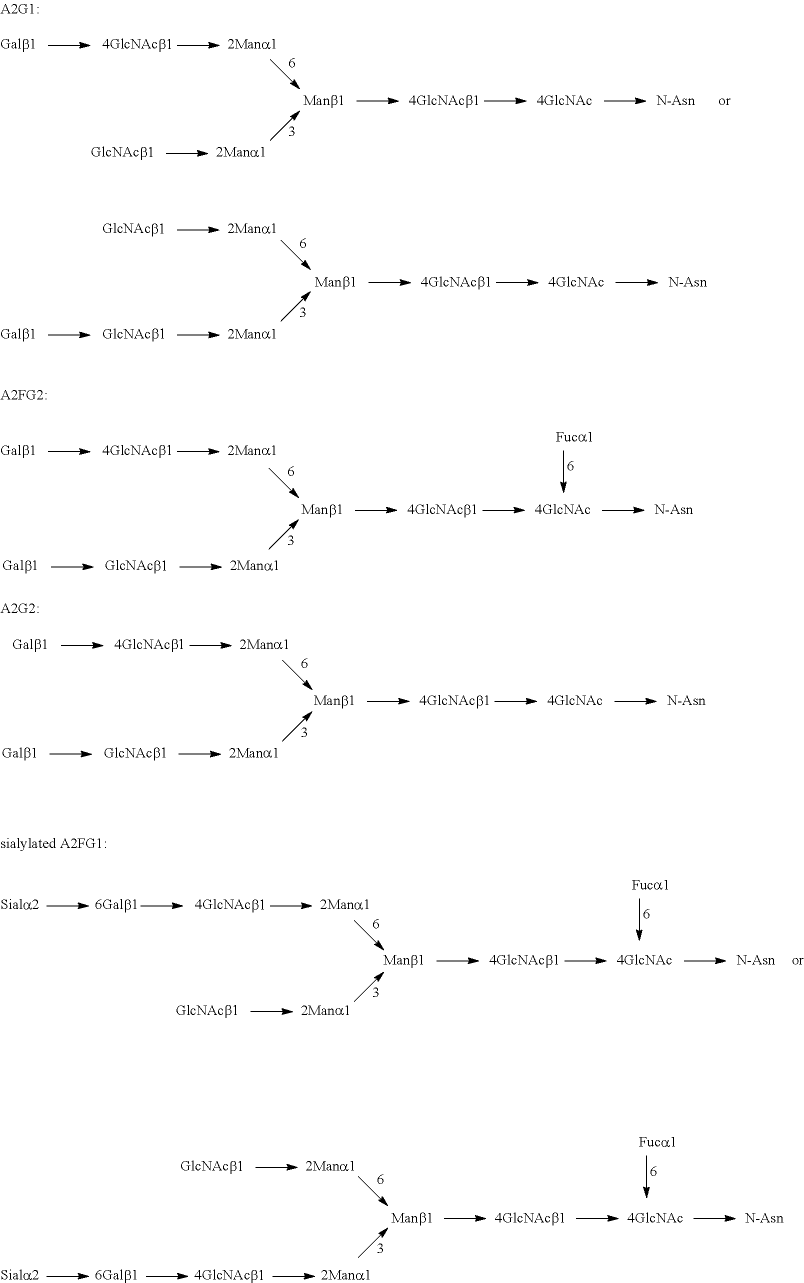

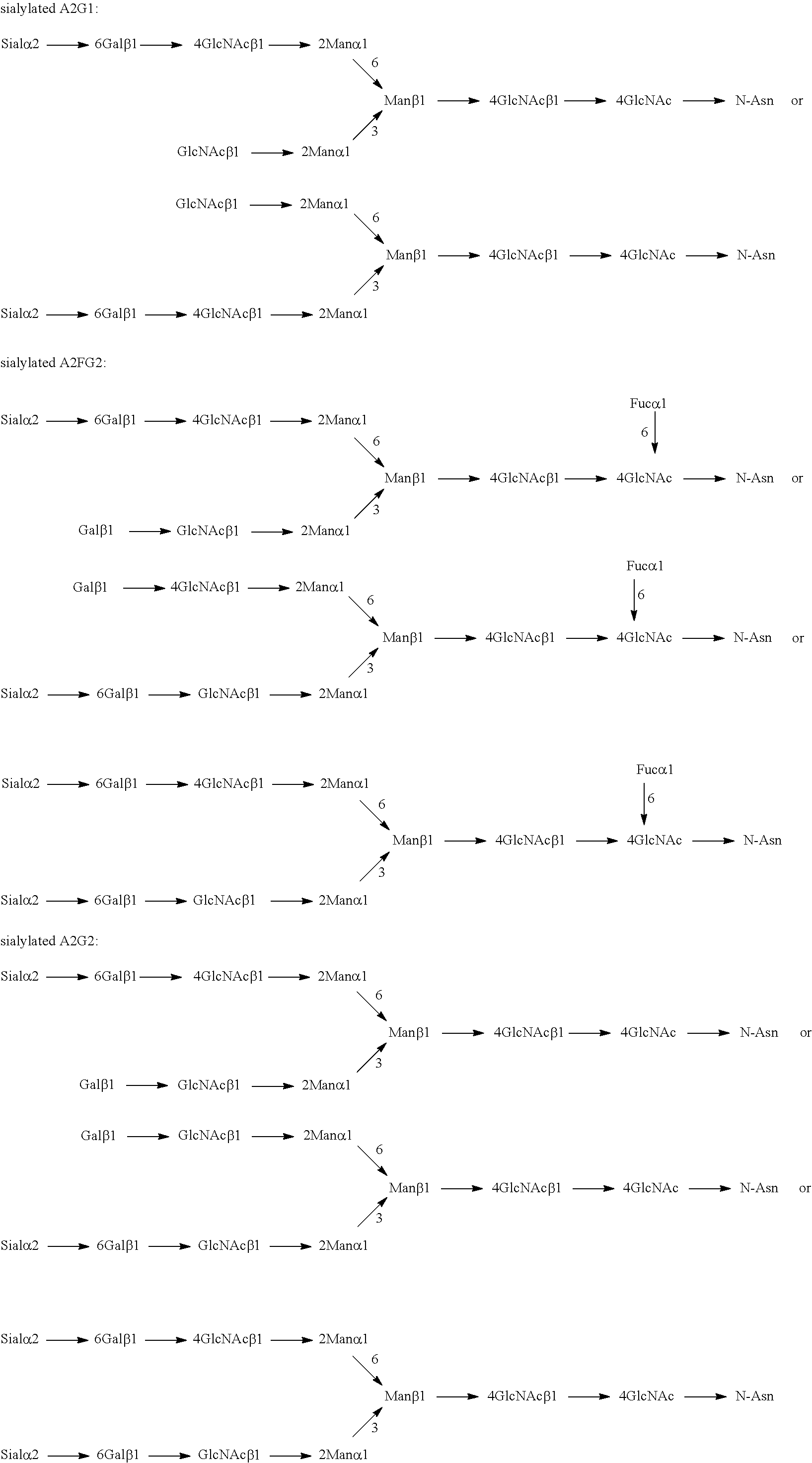

Beside the complex types of oligosaccharides, other examples of N-glycoside linked sugar chains include the high mannose type, in which only mannose binds to the non-reducing terminal of the core structure; a hybrid type, in which the non-reducing terminal side of the core structure has both, branches of the high mannose N-glycoside-linked sugar chain and complex N-glycoside-linked sugar chains; and the like.

Fucose may be found on different sites within the N-glycan tree, yet the vast majority of fucose on antibody attached N-glycans is linked α1,6 to the terminal GlcNAc at the reducing end. Fucose α1,3 linked to the terminal reducing GlcNAc are not found in human derived recombinant proteins but are e.g. produced in plants and hold a risk of severe immunogenicity. Other possible but rarely found linkages of fucose are α1,3 and α1,4 to antennary located GlcNAc or α1,2 to antennary located Gal residues (H-antigen, a substructure of the A and B blood group antigens).

The majority of sugar chains produced in H4-II-E cells described in the present invention are of the “complex biantennary type” and do not contain fucose bound to N-acetylglucosamine at the reducing end.

The term “low fucosylation” means that less than 20% of glycans/glycosidic structures contained in the glycoprotein/contained in the antibody or Fc-fusion protein contain fucose bound to the terminal reducing GlcNAc. More preferred, less than 10% or even more preferred, less than 5% of all complex, biantennary glycans contain fucose bound to the terminal reducing GlcNAc of the glycans. Low fucosylation describes a range between 0% to 20% fucosylation, 0% to 10% fucosylation, preferably 0% to 5% fucosylation. Low fucosylation specifically describes a range between 0.5% to 20% fucosylation, 0.5% to 10% fucosylation, preferably 0.5% to 5% fucosylation. Low fucosylation furthermore describes a range between 1% to 20% fucosylation, 1% to 10% fucosylation, 1% to 5% fucosylation. Therefore, the degree of defucosylation is more than 80%. This means that more than 80% of glycans contain no fucose bound to the terminal reducing GlcNAc. In a specific embodiment more than 90% or preferably more than 95% of glycans contain no fucose bound to the terminal reducing GlcNAc. The degree of defucosylation thus ranges between 80% to 100%, 90% to 100%, preferably 95% to 100%. The degree of defucosylation specifically ranges between 80% to 99.5%, 90% to 99.5%, preferably 95% to 99.5%. The degree of defucosylation preferably ranges between 80% to 99%, 90% to 99%, preferably 95% to 99%.

Having “high galactosylation” means that more than 40% of all glycans/glycosidic structures contained in the glycoprotein/contained in the antibody or Fc-fusion protein of the complex type contain one or two galactose residues linked to GlcNAc residues at the terminal non-reducing ends of the core structure. More preferred, more than 45% or even more preferred, more than 50% of all glycans of the complex biantennary type are galactosylated. High galactosylation describes a range between 40% to 100% galactosylation, 45% to 100% galactosylation, preferably 50% to 100% galactosylation or 51% to 100% galactosylation. High galactosylation specifically describes a range between 40% to 99.5% galactosylation, 45% to 99.5% galactosylation, preferably 50% to 99.5% galactosylation or 51% to 99.5% galactosylation. High galactosylation preferably describes a range between 40% to 99% galactosylation, 45% to 99% galactosylation, preferably 50% to 99% galactosylation or 51% to 99% galactosylation.

High galactosylation preferably describes the presence of at least one galactose residue (G1) in the glycan/glycosidic structure, more preferably one or two galactose residues in the glycan/glycosidic structure (G1 or G2). Preferably high galactosylation means that 50% of the glycosidic structures contain at least one galactose residue. Specifically, high galactosylation means the presence of either G1 or G2 glycosidic structures, but little or no G0 glycosidic structures.

Therefore, the degree of degalactosylation is less than 60%. This means that less than 60% of all glycans of the complex type contain no residues linked to GlcNAc at the terminal non-reducing end of the core structure. In a specific embodiment less than 55% or preferably less than 50% or 49% of all glycans of the complex type contain no galactose residues linked to GlcNAc at the terminal non-reducing end of the core structure. The degree of degalactosylation thus ranges between 60% to 0%, 55% to 0%, preferably 50% to 0% or 49% to 0%. The degree of degalactosylation specifically ranges between 60% to 0.5%, 55% to 0.5%, preferably 50% to 0.5% or 49% to 0.5%. The degree of degalactosylation preferably ranges between 60% to 1%, 55% to 1%, preferably 50% to 1% or 49% to 1%.

Having high sialylation or high activity for adding sialic acid or neuraminic acid residues to galactosylated glycosidic structures such as biantennary glycans, means that more than 5% of all glycans contain terminal sialic acid residues. More preferred, 5-10% or 0-8% or 1-8% of all Fc-glycans contain sialic acid or neuraminic acid residues. In a specific embodiment more than 10% or 10%-50% or 10%-45% of all Fc-glycans are sialylated. Therefore, the degree of glycans not containing terminal sialic acid residues is less than 95%, or it ranges between 95-90% or 92-100%. In a specific embodiment the degree of glycans not containing terminal sialic acid residues is less than 90% or it ranges between 50%-90% or 55%-90%.

The use of a H4-II-E cell as a “host cell” for the production of a recombinant glycoprotein, especially antibodies or Fc-fusion proteins is the subject matter of the present invention. The H4-II-E cell is not a standard host cell.

Standard “host cells” or commonly used host cells for the production of biopharmaceutical proteins in the meaning of the present invention are e.g. BHK21, BHK TK-, CHO, CHO-K1, CHO-DUKX, CHO-DUKX B1, CHO-DG44, murine myeloma cells, preferably NS0 and Sp2/0 cells or the derivatives/progenies of any of such cell line. Particularly preferred standard host cells are CHO-DG44, CHO-DUKX, CHO-K1 and BHK21, and even more preferred CHO-DG44 and CHO-DUKX cells. Most preferred standard host cells are CHO-DG44 cells. Examples of murine and hamster cells are also summarized in Table 1. However, derivatives/progenies of those cells, other mammalian cells, including but not limited to human, mice, rat, monkey, and avian or preferably rodent cell lines, or eukaryotic cells, including but not limited to yeast, insect and plant cells, are also used as standard host cells, particularly for the production of biopharmaceutical proteins. Typically, the cells are capable of expressing and secreting large quantities of a particular glycoprotein of interest into the culture medium.

TABLE 1
Eukaryotic standard host cells/commonly used production cell lines

| CELL LINE | ORDER NUMBER |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (= CHO duk⁻, CHO/dhfr⁻) | |
| CHO-DUKX B11 | ATCC CRL-9010 |
| CHO-DG44 | (Urlaub et al., 1983) |
| CHO Pro-5 | ATCC CRL-1781 |
| CHO Lec13 | |
| CHO Lec10 | |
| FUT8 knock-out | |
| V79 | ATCC CCC-93 |
| B14AF28-G3 | ATCC CCL-14 |
| PER.C6 | (Fallaux et al., 1998) |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |

In general, host cells are most preferred, when being established, adapted, and completely cultivated under serum free conditions, and optionally in media which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma), EX-CELL Media (SAFC), CDM4-CHO and SFM4CHO (HyClone) are exemplary appropriate nutrient solutions. Any of the media may be supplemented as necessary with a variety of compounds examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that would be known to those skilled in the art. In the present invention the use of serum-free medium is preferred, but media supplemented with a suitable amount of serum can also be used for the cultivation of H4-II-E cells and for the growth and selection of stable producer cells. For the growth and selection of genetically modified cells expressing the selectable gene, a suitable selection agent is added to the culture medium.

To adapt cells grown in serum-containing media to serum-free growth and to culture cells in single cell suspension cultures, serum-free media are used. To facilitate growth in suspension, serum-free and Calcium ion free or Calcium-reduced media are preferred. According to the present invention, H4-II-E cells are adapted to and subsequently cultured in suspension in serum-free and Ca-reduced media. A method to adapt and culture H4-II-E cells in suspension and serum-free media comprises the use of Serum-free and Calcium ion (Ca2+)-free or Calcium-reduced media. The term calcium-reduced or preferably calcium-free medium means media that are defined to contain 1 μmol/L-500 μmol/L of Ca2+ ions, more preferred Calcium-reduced media contain 1 μmol/L-250 μmol/L of Ca2+ ions, and even more preferred, Calcium-reduced or Ca-free media contain 0 μmol/L-100 μmol/L or 0.5 μmol/L-100 μmol/L of Ca2+ ions. Examples of commercially available Calcium-free media are MEM Joklik Modification (Sigma) or MEM Spinner Modification (Sigma). A typical Calcium-containing medium used for the adherent cultivation of H4-II-E cells in the presence of serum is Eagle's Minimum Essential Medium (Sigma) which contains 1360 μmol/L of Ca2+ ions. The term “host cell” according to the present invention encompasses a cell comprising a heterologous nucleic acid sequence as well as a cell not (yet) comprising a heterologous nucleic acid sequence. In a specific embodiment of the present invention said host cell comprises a heterologous nucleic acid sequence, which encodes a (recombinant) protein, preferably a glycoprotein of interest, e.g. an antibody or Fc-fusion protein, which is expressed by said host cell. To this effect, said host cell is cultivated under conditions which allow for the expression of said (recombinant) protein.

The term “protein” is used interchangeably with amino acid residue sequences or polypeptide and refers to polymers of amino acids of any length. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with like properties.

The term “polypeptide” means a sequence with more than 10 amino acids and the term “peptide” means sequences up to 10 amino acids length.

The term “nucleic acid sequence”, “gene of interest” (GOI), “selected sequence”, or “product gene” have the same meaning herein and refer to a polynucleotide sequence of any length that encodes a “product of interest” or “protein of interest”, also mentioned by the term “desired product”. In a preferred embodiment of the present invention the nucleic acid sequence or gene of interest encodes a glycoprotein, preferably an antibody or Fc fusion protein. The nucleic acid sequence or gene of interest can be full length or a truncated gene, a fusion or tagged gene, and can be a cDNA, a genomic DNA, or a DNA fragment, preferably, a cDNA. It can be the native sequence, i.e. naturally occurring form(s), or can be mutated or otherwise modified as desired. These modifications include codon optimizations to optimize codon usage in the selected host cell, humanization or tagging. The selected sequence can encode a secreted, cytoplasmic, nuclear, membrane bound or cell surface polypeptide.

The “glycoprotein of interest” includes proteins, polypeptides, fragments thereof, peptides, all of which can be expressed in the H4-II-E host cell. Desired proteins can be for example antibodies, enzymes, cytokines, lymphokines, adhesion molecules, receptors and derivatives or fragments thereof, and any other polypeptides that can serve as agonists or antagonists and/or have therapeutic or diagnostic use. Examples for a desired protein/polypeptide are also given below.

In the case of more complex molecules such as monoclonal antibodies the gene of interest encodes one or both of the two antibody chains.

The term “antibody” refers to a protein consisting of one or more polypeptides substantially encoded by immunoglobulin genes. The recognized immunoglobulin genes include the kappa, lambda, alpha, gamma, delta, epsilon and mu constant regions genes as well as the myriad immunoglobulin variable region genes.

As used herein, the term “antibody” includes a polyclonal, monoclonal, bi-specific, multi-specific, human, humanized, or chimeric antibody.

The terms “antibody” and “immunoglobulin” are used interchangeably and are used to denote glycoproteins having the structural characteristics noted above for immunoglobulins.

The term “antibody” is used in the broadest sense and specifically covers single monoclonal antibodies (including agonist and antagonist antibodies) and antibody compositions with polyepitopic specificity. The term “antibody” specifically covers monoclonal antibodies (including full length monoclonal antibodies), polyconal antibodies, multispecific antibodies (e.g. bispecific antibodies) and antibody fragments so long as they contain or are modified to contain at least the portion of the CH2 domain of the heavy chain immunoglobulin constant region comprising the N-linked glycosylation site. Exemplary antibodies within the scope of the present invention include but are not limited to anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 or anti-IgE antibodies.

The term “monoclonal antibody” (mAb) as used herein refers to an antibody obtained from a population of substantially homogeneous antibodies based on the amino acid sequence. Monoclonal antibodies are highly specific, being direct against a single antigenic site. Furthermore, in contrast to conventional (polyclonal) antibody preparations which typically include different antibodies directed against different determinants (epitopes), each mAb is directed against a single determinant on the antigen. In addition to their specificity, the mAbs are advantageous in that they can be synthesized by cell culture (hybridomas, recombinant cells or the like) uncontaminated by other immunoglobulins. The mAbs herein include chimeric, humanized and human antibodies.

“Chimeric antibodies” are antibodies whose light and/or heavy chain genes have been constructed, typically by genetic engineering, from immunoglobulin variable and constant regions belonging to identical or homologous to corresponding sequences of different species, such as mouse and human. Or alternatively, whose heavy chain genes are belong to a particular antibody class or subclass while the remainder of the chain is from another antibody class or subclass of the same or from another species. It covers also fragments of such antibodies, so long as they contain or are modified to contain at least one CH2 domain. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant or effector domain from a human antibody (e.g. ATCC Accession No. CRL 9688 secretes an anti-Tac chimeric antibody), although other mammalian species may be used.

The term “humanized antibodies” according to the present invention refers to specific chimeric antibodies, immunoglobulin chains or fragments thereof (such as Fv, Fab, Fab′, F(ab)₂ or other antigen-binding subsequences of antibodies) so long as they contain or are modified to contain at least the portion of the CH2 domain of the heavy chain immunoglobulin constant region comprising the N-linked glycosylation site, and which contain minimal sequence derived from non-human immunoglobulin. For the most part, humanized antibodies are human immunoglobulins (recipient antibody) in which residues from a complementary—determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by the corresponding non-human residues. Furthermore, humanized antibodies can comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all off the CDR regions correspond to those of a non-human immunoglobulin and all or substantially all of the framework regions are those of a human immunoglobulin consensus sequence. The humanized antibody optimally also will comprise at least a portion of an immunoglobulin constant region, typically that of a human immunoglobulin.

humanized antibody: comprising a human framework region and one or more CDRs from a non-human (usually a mouse or rat) antibody. Adjustments in framework amino acids might be required to keep antigen binding specificity, affinity and or structure of domain.

The term “CH2 domain” according to the present invention is meant to describe the CH2 domain of the heavy chain immunoglobulin constant region comprising the N-linked glycosylation site. In defining an immunoglobulin CH2 domain reference is made to immunoglobulins in general and in particular to the domain structure of immunoglobulins as applied to human IgG1 by Kabat, E. A. (Kabat, 1988; Kabat et al., 1991). Accordingly, immunoglobulins are generally heterotetrameric glycoproteins of about 150 kDa, composed of two identical light and two identical heavy chains. Each light chain is linked to a heavy chain by one covalent disulfide bond, while the number of disulfide linkages varies between the heavy chains of different immunoglobulins isotypes. Each heavy and light chain also has regularly spaced intrachain disulfide bridges. Each heavy chain has an amino terminal variable domain (VH) followed by carboxy terminal constant domains (CH). Each light chain has a variable N-terminal domain (VL) and a C-terminal constant domain (CL).

Depending on the amino acid sequence of the constant domain of the heavy chains, antibodies can be assigned to different classes. There are five major classes: IgA, IgD, IgE, IgG and IgM. The heavy chain constant domains that correspond to the different classes of antibodies are called alpha, delta, epsilon, gamma and mu domains, respectively. The mu chain of IgM contains five domains (VH, CHmu1, CHmu2, CHmu3 and CHmu4). The heavy chain of IgE also contains five domains while the heavy chain of IgA has four domains. The immunoglobulin class can be further divided into subclasses (isotypes), e.g. IgG1, IgG2, IgG3, IgG4, IgA1 and IgA2.

The subunit structures and three-dimensional configuration of different classes of immunoglobulins are well known. Of these IgA and IgM are polymeric and each subunit contains 2 light and two heavy chains. The heavy chain of IgG contains a length of polypeptide chain lying between the CHgamma1 and CHgamma2 domains known as the hinge region. The alpha chain of IgA has a hinge region containing an O-linked glycosylation site and the mu and epsilon chains do not have a sequence analogous to the hinge region of the gamma and alpha chains, however, they contain a fourth constant domain lacking in the others.

A CH2 domain therefore is an immunoglobulin heavy chain constant region domain. The Fc region of a full antibody usually comprises two CH2 domains and two CH3 domains. According to the present invention, the CH2 domain is preferably the CH2 domain of one of the five immunoglobulin classes indicated above. Preferred are mammalian immunoglobulin CH2 domains such as primate or murine immunoglobulin with the primate and especially human immunoglobulin CH2 domains being preferred. The amino acid sequences of immunoglobulin CH2 domains are known or are generally available to the skilled artisan (Kabat et al., 1991). A preferred immunoglobulin CH2 domain within the context of the present invention is a human IgG and preferably from IgG1, IgG2, IgG3, IgG4, more preferably a human IgG1 and IgG3 and even more preferred a human IgG1. Using the numbering system of Edelman (Edelman et al., 1969), the immunoglobulin CH2 domain preferably begins at amino acid position equivalent to glutamine 233 of human IgG1 and extends through amino acid equivalent to lysine 340 (Ellison and Hood, 1982).

With respect to human antibody molecules reference is made to the IgG class in which an N-linked oligosaccharide is attached to the amide side chain of Asn 297 of the beta-4 bend to the inner face of the CH2 domain of the Fc region. It is characteristic of the glycoprotein, especially the antibody or Fc-fusion protein of the present invention that it contains or be modified to contain at least a CH2 domain. The CH2 domain is a CH2 domain of an immunoglobulin having a single N-linked oligosaccharide of a human IgG CH2 domain. The CH2 domain is preferably the CH2 domain of human IgG1.

The “glycoproteins of interest”, “polypeptide of interest”, “protein of interest” or “product of interest” are those mentioned above and include antibodies or Fc-fusion proteins all of which can be expressed in the H4-II-E host cell. Furthermore, desired proteins or glycoproteins of interest can be for example enzymes, cytokines, lymphokines, adhesion molecules, receptors and derivatives or fragments thereof, and any other polypeptides that can serve as agonists or antagonists and/or have therapeutic or diagnostic use, which is glycosylated.

Especially, desired glycoproteins/polypeptides or proteins of interest are for example, but not limited to insulin, insulin-like growth factor, hGH, tPA, cytokines, such as interleukines (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF), such as TNF alpha and TNF beta, TNF gamma, TRAIL; G-CSF, GM-CSF, M-CSF, MCP-1, VEGF and nanobodies. Also included is the production of erythropoietin or any other hormone growth factors and any other polypeptides that can serve as agonists or antagonists and/or have therapeutic or diagnostic use. The H4-II-E cell according to the invention can be advantageously used for production of antibodies such as monoclonal, polyclonal, multispecific antibodies, or fragments thereof which comprise a CH2 domain, Fc and Fc′-fragments, heavy and light immunoglobulin chains and their constant fragments. Furthermore, the method for producing a (recombinant) glycoprotein according to the invention can be advantageously used for production of antibodies such as monoclonal, polyclonal, multispecific antibodies, or fragments thereof which comprise a CH2 domain, Fc and Fc′-fragments, heavy and light immunoglobulin chains and their constant fragments as well as Fc-fusion proteins.

“Fc-fusion proteins” are defined as proteins which contain or are modified to contain at least the portion of the CH2 domain of the heavy chain immunoglobulin constant region comprising the single N-linked glycosylation site. According to the Kabat EU nomenclature (Kabat et al., 1991) this position is Asn297 in an IgG1, IgG2, IgG3 or IgG4 antibody.

The other part of the fusion protein can be the complete sequence or any part of the sequence of a natural or modified heterologous protein or a composition of complete sequences or any part of the sequence of natural or modified heterologous protein proteins. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG3. Examples of Fc fusion proteins comprise MCP1-Fc, ICAM-Fc, EPO-Fc, scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, effector domains. Thus, a Fc fusion protein according to the present invention comprises also a single chain Fv fragment linked to the CH2 domain of the heavy chain immunoglobulin constant region comprising the N-linked glycosylation site.

The glycoprotein of interest, especially the antibody or Fc-fusion protein is preferably recovered/isolated from the culture medium as a secreted polypeptide, or it can be recovered/isolated from host cell lysates if expressed without a secretory signal. It is necessary to purify the glycoprotein of interest, especially the antibody or Fc-fusion protein from other recombinant proteins and host cell proteins in a way that substantially homogenous preparations of the protein of interest are obtained. As a first step, cells and/or particulate cell debris are removed from the culture medium or lysate for example by centrifugation or filtration. The glycoprotein of interest, especially the antibody or Fc-fusion protein, thereafter purified from contaminant soluble proteins, polypeptides and nucleic acids, for example, by fractionation on immunoaffinity or ion-exchange columns, ethanol precipitation, ammonium sulfate precipitation, reverse phase HPLC, chromatofocusing, Sephadex chromatography, chromatography on silica or on a cation exchange resin such as DEAE, gel filtration or specifically by protein A affinity chromatography. In general, methods teaching a skilled person how to purify a protein heterologous expressed by host cells, are well known in the art. By definition any sequences or genes introduced into a host cell are called “heterologous sequences” or “heterologous genes” or “transgenes” with respect to the host cell, even if the introduced sequence or gene is identical to an endogenous sequence or gene in the host cell. A “heterologous” protein is thus a protein expressed from a heterologous sequence.

The term “recombinant” is used exchangeably with the term “heterologous” throughout the specification of this present invention, especially in the context with protein expression. Thus, a “recombinant” protein is a protein expressed from a heterologous sequence.

Heterologous gene sequences can be introduced into a target cell by using an “expression vector”, preferably a eukaryotic, and even more preferably a mammalian expression vector. Methods used to construct vectors are well known to a person skilled in the art and described in various publications. In particular techniques for constructing suitable vectors, including a description of the functional components such as promoters, enhancers, termination and polyadenylation signals, selection markers, origins of replication, and splicing signals, are reviewed in considerable details in (Sambrook et al., 1989) and references cited therein. Vectors may include but are not limited to plasmid vectors, phagemids, cosmids, articificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif.

The prerequisite for stable expression of a (glyco)protein of interest, preferably an antibody or Fc fusion protein, in H4-II-E rat hepatoma cells is that the cells are transfected with a nucleic acid sequence encoding the protein of interest wherein said nucleic acid sequence is operatively linked to at least one regulatory sequence allowing for expression of said nucleic acid sequence. The prerequisite for stable glycoprotein expression in H4-II-E cells is that the cells are transfected with a DNA in which the gene encoding the protein of interest is functionally linked to genetic elements controlling gene transcription.

Such elements controlling gene transcription or “regulatory sequences” are for example enhancer, promoter and 5′UTR sequences. Examples include but are not limited to the SV40 enhancer, CMV enhancer, albumin enhancer, hepatitis B enhancer, aldolase enhancer, Ig enhancer, tyrosinase enhancer, SV40 promoter, EF1-alpha promoter, chicken beta-actin promoter, CMV promoter, HSV TK promoter, Phosphoglycerokinase (PGK) promoter, Polymerase II promoter, Ubiquitin C promoter, albumin promoter, alpha1-antitrypsin promoter, alpha-fetoprotein (AFP) promoter, aldolase promoter, alpha1 microglobulin promoter, phosphoenolpyruvate carboxykinase promoter, RSV promoter, GAPDH promoter, beta globin promoter, and MT1 Promoter.

Therefore, in a preferred embodiment the H4-II-E cell of the present invention comprises an expression vector comprising at least one nucleic acid sequence which is a regulatory sequence necessary for transcription and translation of a nucleic acid sequence that encodes a glycoprotein of interest. In a specific embodiment, the expression vector comprises at least one regulatory sequence allowing the transcription and translation (expression) of the nucleic acid sequence or gene of interest encoding the glycoprotein of interest, which is preferably an antibody or Fc fusion protein.

“Regulatory sequences” furthermore include promoters, enhancers, termination and polyadenylation signals, and other expression control elements. Both inducible and constitutive regulatory sequences are known in the art to function in various cell types. Transcriptionally regulatory elements normally comprise a promoter upstream of the gene sequence to be expressed, transcriptional initiation and termination sites, and a polyadenylation signal sequence. The term transcriptional initiation site refers to the nucleic acid in the construct corresponding to the first nucleic acid incorporated into the primary transcript, i.e., the mRNA precursor; the transcriptional initiation site may overlap with the promoter sequences. The term transcriptional termination site refers to a nucleotide sequence normally represented at the 3′end of a gene of interest or the stretch of sequences to be transcribed, that causes RNA polymerase to terminate transcription. The polyadenylation signal sequence or poly-A addition signal provides the signal for the cleavage at a specific site at the 3′end of eukaryotic mRNA and the post-transcriptional addition in the nucleus of a sequence of about 100-200 adenine nucleotides (polyA tail) to the cleaved 3′end. The polyadenylation signal sequence includes the sequence AATAAA located at about 10-30 nucleotides upstream from the site of cleavage, plus a downstream sequence. Various polyadenylation elements are known, e.g. SV40 late and early polyA, or BGH polyA. Translational regulatory elements include a translational initiation site (AUG), stop codon and poly A signal for each individual polypeptide to be expressed. An internal ribosome entry site (IRES) is included in some constructs. IRES is defined below. In order to optimize expression it may be necessary to remove, add or alter 5′ and/or 3′untranslated portions of the nucleic acid sequence to be expressed to eliminate potentially extra inappropriate alternative translation initiation codons or other sequences that may interfere with or reduce expression, either at the level of transcription or translation. Alternatively consensus ribosome binding sites can be inserted immediately 5′ of the start codon to enhance expression. To produce a secreted polypeptide, the selected sequence will generally include a signal sequence encoding a leader peptide that directs the newly synthesized polypeptide to and through the ER membrane where the polypeptide can be routed for secretion. The leader peptide is often but not universally at the amino terminus of a secreted protein and is cleaved off by signal peptidases after the protein crosses the ER membrane. The selected sequence will generally, but not necessarily, include its own signal sequence. Where the native signal sequence is absent, a heterologous signal sequence can be fused to the selected sequence. Numerous signal sequences are known in the art and available from sequence databases such as GenBank and EMBL.

A “promoter” refers to a polynucleotide sequence/a nucleic acid sequence that controls transcription of a gene or sequence to which it is operatively linked. A promoter includes signals for RNA polymerase binding and transcription initiation. A promoter used will be functional in the cell type of the host cell in which expression of the selected sequence is contemplated, which is in the present invention the H4-II-E cell. A large number of promoters, including constitutive, inducible and repressible promoters from a variety of different sources, are well known in the art (and identified in databases such as GenBank) and are available as or within cloned polynucleotides (from, e.g. depositories such as ATCC as well as other commercial or individual sources). With inducible promoters, the activity of the promoter increases or decreases in response to a signal. For example, the tetracycline (tet) promoter containing the tetracycline operator sequence (tetO) can be induced by a tetracycline-regulated transactivator protein (tTA). Binding of the tTA to the tetO is inhibited in the presence of tet. For other inducible promoters including jun, fos, metallothionein and heat shock promoters, see, e.g., Sambrook et al., 1989. Among the eukaryotic promoters that have been identified as strong promoters for high-level expression are the SV40 early promoter, adenovirus major late promoter, mouse methallothionein-I promoter, Rous sarcoma virus long terminal repeat and human cytomegalovirus immediate early promoter (CMV). Other heterologous mammalian promoters include, e.g., actin promoter, immunoglobulin promoter, heat-shock promoters. The aforementioned promoters are well known in the art.

An “enhancer”, as used herein, refers to a polynucleotide sequence/a nucleic acid sequence that acts on a promoter to enhance transcription of a gene or coding sequence to which it is operatively linked. Unlike promoters, enhancers are relatively orientation and position independent and have been found 5′or 3′to the transcription unit, within an intron as well as within the coding sequence itself. Therefore, enhancers may be placed upstream or downstream from the transcription initiation site or at considerable distances from the promoter, although in practice enhancers may overlap physically and functionally with promoters. A large number of enhancers from a variety of different sources are well known in the art (and identified in databases such as GenBank, e.g. SV40 enhancer, CMV enhancer, polyoma enhancer, adenovirus enhancer) and available as or within cloned polynucleotide sequences (from, e.g., depositories such as the ATCC as well as other commercial or individual sources). A number of polynucleotides comprising promoter sequences (such as the commonly used CMV promoter) also comprise enhancer sequences. For example, all of the strong promoters listed above also contain strong enhancers.

The term “operatively linked” means that two or more nucleic acid sequences or sequence elements are positioned in a way that permits them to function in their intended manner. For example, a promoter and/or enhancer is operatively linked to a coding sequence if it acts in cis to control or modulate the transcription of the linked sequence. Generally, but not necessarily, the DNA sequences that are operatively linked are contiguous and, where necessary to join two protein coding regions or in the case of a secretory leader, contiguous and in reading frame.

However, although an operatively linked promoter is generally located upstream of the coding sequence, it is not necessarily contiguous with it. Enhancers do not have to be contiguous as long as they increase the transcription of the coding sequence. For this they can be located upstream or downstream of the coding sequence and even at some distance. A polyadenylation site is operatively linked to a coding sequence if it is located at the 3′end of the coding sequence in a way that transcription proceeds through the coding sequence into the polyadenylation signal. Linking is accomplished by recombinant methods known in the art, e.g. using PCR methodology, by ligation at suitable restrictions sites or by annealing. Synthetic oligonucleotide linkers or adaptors can be used in accord with conventional practice if suitable restriction sites are not present.

A “transcription unit” defines a region within a construct that contains one or more genes to be transcribed, wherein the genes contained within the segment are operatively linked to each other and transcribed from a single promoter, and as result, the different genes are at least transcriptionally linked. More than one protein or product can be transcribed and expressed from each transcription unit. Each transcription unit will comprise the regulatory elements necessary for the transcription and translation of any of the selected sequence that are contained within the unit.

The term “expression” as used herein refers to transcription and/or translation of a heterologous nucleic acid sequence within a host cell. The level of expression of a desired product/protein of interest in a host cell may be determined on the basis of either the amount of corresponding mRNA that is present in the cell, or the amount of the desired polypeptide/protein of interest encoded by the selected sequence as in the present examples. For example, mRNA transcribed from a selected sequence can be quantitated by Northern blot hybridization, ribonuclease RNA protection, in situ hybridization to cellular RNA or by PCR. Proteins encoded by a selected sequence can be quantitated by various methods, e.g. by ELISA, by Western blotting, by radioimmunoassays, by immunoprecipitation, by assaying for the biological activity of the protein, by immunostaining of the protein followed by FACS analysis or by homogeneous time-resolved fluorescence (HTRF) assays.

An “expression cassette” defines a region within a construct that contains one or more genes to be transcribed, wherein the genes contained within the segment are operatively linked to each other and transcribed from a single promoter, and as result, the different genes are at least transcriptionally linked. More than one protein or product can be transcribed and expressed from each transcription unit. Each transcription unit will comprise the regulatory elements necessary for the transcription and translation of any of the selected sequence that are contained within the unit.

“Transfection” of eukaryotic host cells with a polynucleotide or expression vector, resulting in genetically modified cells or transgenic cells, can be performed by any method well known in the art (see e.g. (Sambrook et al., 1989)). Transfection methods include but are not limited to liposome-mediated transfection, calcium phosphate co-precipitation, electroporation, nucleofection, nucleoporation, microporation, polycation (such as DEAE-dextran)-mediated transfection, protoplast fusion, viral infections and microinjection. Preferably, the transfection is a stable transfection. The transfection method that provides optimal transfection frequency and expression of the heterologous genes in the particular host cell line and type is favoured. Suitable methods can be determined by routine procedures. For stable transfectants the constructs are either integrated into the host cell's genome or an artificial chromosome/mini-chromosome or located episomally so as to be stably maintained within the host cell.

A “selectable marker gene” or “selection marker gene” is a gene which allows the specific selection of cells which contain this gene by the addition of a corresponding selecting agent to the cultivation medium. As an illustration, an antibiotic resistance gene may be used as a positive selectable marker. Only cells which have been transformed with this gene are able to grow in the presence of the corresponding antibiotic and are thus selected. Untransformed cells, on the other hand, are unable to grow or survive under these selection conditions. There are positive, negative and bifunctional selectable markers. Positive selectable markers permit the selection and hence enrichment of transformed cells by conferring resistance to the selecting agent or by compensating for a metabolic or catabolic defect in the host cell. By contrast, cells which have received the gene for the selectable marker can be selectively eliminated by negative selectable markers. An example of this is the thymidine kinase gene of the Herpes Simplex virus, the expression of which in cells with the simultaneous addition of acyclovir or gancyclovir leads to the elimination thereof. The selectable markers used in this invention, including the amplifiable selectable markers, include genetically modified mutants and variants, fragments, functional equivalents, derivatives, homologues and fusions with other proteins or peptides, provided that the selectable marker retains its selective qualities. Such derivatives display considerable homology in the amino acid sequence in the regions or domains which are deemed to be selective. The literature describes a large number of selectable marker genes including bifunctional (positive/negative) markers (see for example WO 92/08796 and WO 94/28143). Examples of selectable markers which are usually used in eukaryotic cells include the genes for aminoglycoside phosphotransferase (APH), hygromycine phosphostransferase (HYG), dihydrofolate reductase (DHFR), thymidine kinase (TK), glutamine synthetase, asparagin synthetase and genes which confer resistance to neomycin (G418/Geneticin), puromycin, histidinol D, bleomycin, phleomycin and zeocin.

Selection may be made by fluorescence activated cell sorting (FACS) using for example a cell surface marker, bacterial β-galactosidase or fluorescent proteins (e.g. green fluorescent proteins (GFP) and their variants from Aequorea victoria and Renilla reniformis or other species; red fluorescent proteins, fluorescent proteins and their variants from non-bioluminescent species (e.g. Discosoma sp., Anemonia sp., Clavularia sp., Zoanthus sp.) to select for recombinant cells.

The term “selection agent” refers to a substance that interferes with the growth or survival of a H4-II-E cell that is deficient in a particular selectable gene. For example, to select for the presence of an antibiotic resistance gene like APH (aminoglycoside phosphotransferase) in a transfected cell the antibiotic Geneticin (G418) is used.

The term “modified neomycin-phosphotransferase (NPT)” covers all the mutants described in WO2004/050884, particularly the mutant D227G (Asp227Gly), which is characterised by the substitution of aspartic acid (Asp, D) for glycine (Gly, G) at amino acid position 227 and particularly preferably the mutant F240I (Phe240Ile), which is characterised by the substitution of phenylalanine (Phe, F) for isoleucine (Ile, I) at amino acid position 240.

The present invention further includes a method of preparing and selecting recombinant H4-II-E cells which comprises the following steps: (i) transfecting the H4-II-E cells with genes which code for at least one glycoprotein/product of interest and a selection marker, preferably neomycin-phosphotransferase, wherein in order to enhance the transcription or expression at least the gene (or genes) of interest are linked to enhancer, promoter and 5′-UTR sequences driving the stable expression of the genes and/or is optionally functionally linked to at least one TE element (see WO2008/012142, which is incorporated herein by reference); (ii) cultivating the cells under conditions that enable expression of the different genes; and (iii) selecting these co-integrated genes by cultivating the cells in the presence of a selecting agent such as e.g. G418, MTX or MSX. Preferably, the transfected cells are cultivated in medium in the absence of serum. Preferably the concentration of G418 is at least 200 μg/mL. However, the concentration may also be at least 400 μg/mL.

### Amplifiable Selectable Marker Gene:

In addition, the cells according to the invention may optionally also be subjected to one or more gene amplification steps in which they are cultivated in the presence of a selecting agent which leads to amplification of an amplifiable selectable marker gene.

The prerequisite is that the H4-II-E cells are additionally transfected with a gene which codes for an amplifiable selectable marker. It is conceivable for the gene which codes for an amplifiable selectable marker to be present on one of the expression vectors according to the invention or to be introduced into the host cell by means of another vector.

The amplifiable selectable marker gene usually codes for an enzyme which is needed for the growth of eukaryotic cells under certain cultivation conditions. For example, the amplifiable selectable marker gene may code for dihydrofolate reductase (DHFR) or glutamine synthetase (GS). In this case the gene is amplified if a host cell transfected therewith is cultivated in the presence of the selecting agent methotrexate (MTX) or methionine sulphoximine (MSX).

The following Table 2 gives examples of amplifiable selectable marker genes and the associated selecting agents which may be used according to the invention, which are described in an overview by Kaufman (Kaufman, 1990).

TABLE 2
Amplifiable selectable marker genes

| Amplifiable selectable marker gene | Accession number | Selecting agent |
|---|---|---|
| dihydrofolate reductase | M19869 (hamster) | methotrexate (MTX) |
| | E00236 (mouse) | |
| metallothionein | D10551 (hamster) | cadmium |
| | M13003 (human) | |
| | M11794 (rat) | |
| CAD (carbamoylphosphate | M23652 (hamster) | N-phosphoacetyl-L-aspartate |
| synthetase:aspartate | D78586 (human) | |
| transcarbamylase: | | |
| dihydroorotase) | | |
| adenosine-deaminase | K02567 (human) | Xyl-A- or adenosine, |
| | M10319 (mouse) | 2′deoxycoformycin |
| AMP (adenylate)-deaminase | D12775 (human) | adenine, azaserin, coformycin |
| | J02811 (rat) | |
| UMP-synthase | J03626 (human) | 6-azauridine, pyrazofuran |
| IMP 5′-dehydrogenase | J04209 (hamster) | mycophenolic acid |
| | J04208 (human) | |
| | M33934 (mouse) | |
| xanthine-guanine- | X00221 (E. coli) | mycophenolic acid with |
| phosphoribosyltransferase | | limiting xanthine |
| mutant HGPRTase or mutant | J00060 (hamster) | hypoxanthine, aminopterine |
| thymidine-kinase | M13542, K02581 (human) | and thymidine (HAT) |
| | J00423, M68489(mouse) | |
| | M63983 (rat) | |
| | M36160 (Herpes virus) | |
| thymidylate-synthetase | D00596 (human) | 5-fluorodeoxyuridine |
| | M13019 (mouse) | |
| | L12138 (rat) | |
| P-glycoprotein 170 (MDR1) | AF016535 (human) | several drugs, e.g. |
| | J03398 (mouse) | adriamycin, vincristin, |
| | | colchicine |
| ribonucleotide reductase | M124223, K02927 | aphidicoline |
| | (mouse) | |
| glutamine-synthetase | AF150961 (hamster) | methionine sulphoximine |
| | U09114, M60803 (mouse) | (MSX) |
| | M29579 (rat) | |
| asparagine-synthetase | M27838 (hamster) | β-aspartylhydroxamate, |
| | M27396 (human) | albizziin, 5′azacytidine |
| | U38940 (mouse) | |
| | U07202 (rat) | |
| argininosuccinate-synthetase | X01630 (human) | canavanin |
| | M31690 (mouse) | |
| | M26198 (bovine) | |
| ornithine-decarboxylase | M34158 (human) | α-difluoromethylornithine |
| | J03733 (mouse) | |
| | M16982 (rat) | |
| HMG-CoA-reductase | L00183, M12705 (hamster) | compactin |
| | M11058 (human) | |
| N-acetylglucosaminyl- | M55621 (human) | tunicamycin |
| transferase | | |
| threonyl-tRNA-synthetase | M63180 (human) | borrelidin |
| Na⁺K⁺-ATPase | J05096 (human) | ouabain |
| | M14511 (rat) | |

According to the invention the amplifiable selectable marker gene used is preferably a gene which codes for a polypeptide with the function of GS or DHFR.

The term “transformation” or “to transform”, “transfection” or “to transfect” as used herein means any introduction of a nucleic acid sequence into a cell, resulting in genetically modified, recombinant, transformed or transgenic cells. The introduction can be performed by any method well known in the art. Methods include but are not limited to lipofection, electroporation, polycation (such as DEAE-dextran)-mediated transfection, protoplast fusion, viral infections and microinjection or may be carried out by means of the calcium method, electroshock method, intravenous/intramusuclar injection, aerosol inhalation or an oocyte injection. The transformation may result in a transient or stable transformation of the host cells. The term “transfection” or “to transfect”, “transformation” or “to transform” also means the introduction of a viral nucleic acid sequence in a way which is for the respective virus the naturally one. The viral nucleic acid sequence needs not to be present as a naked nucleic acid sequence but may be packaged in a viral protein envelope. Thus, the term relates not only to the method which is usually known under the term “transfection” or “to transfect”, “transformation” or “to transform”. Transfection methods that provide optimal transfection frequency and expression of the introduced nucleic acid are favored. Suitable methods can be determined by routine procedures. For stable transfectants the constructs are either integrated into the host cell's genome or an artificial chromosome/mini-chromosome or located episomally so as to be stably maintained within the host cell.

### SPECIFIC EMBODIMENTS

The present invention describes a rat hepatoma cell comprising a nucleic acid sequence encoding an antibody or Fc-fusion protein, whereby said nucleic acid sequence is operatively linked to at least one regulatory sequence allowing for expression of said nucleic acid sequence encoding an antibody or Fc-fusion protein. The present invention further describes a rat hepatoma cell characterized by carrying a nucleic acid sequence encoding an antibody or Fc-fusion protein, whereby said nucleic acid sequence is operatively linked to at least one regulatory sequence allowing for expression of said nucleic acid sequence encoding an antibody or Fc-fusion protein.

In a specific embodiment the rat hepatoma cell is a H4-II-E cell. In a further specific embodiment the rat hepatoma cell is a cell deposited with the DSMZ under the accession number DSM ACC3129 (H4-II-E) or DSM ACC3130 (H4-II-Es).

The present invention describes a H4-II-E rat hepatoma cell comprising/characterized by carrying a nucleic acid sequence encoding an antibody or Fc-fusion protein, whereby said nucleic acid sequence is operatively linked to at least one regulatory sequence allowing for expression of said nucleic acid sequence encoding an antibody or Fc-fusion protein.

The present invention describes a H4-II-E rat hepatoma cell genetically modified by introducing a nucleic acid sequence/a gene of interest encoding a glycoprotein, preferably an antibody or Fc fusion protein operatively linked to at least one regulatory sequence allowing for expression of said nucleic acid sequence/gene of interest encoding an antibody or Fc-fusion protein.

The present invention describes a H4-II-E rat hepatoma cell or derivatives or progenies thereof comprising/characterized by carrying a nucleic acid sequence encoding an antibody or Fc-fusion protein, whereby said nucleic acid sequence is operatively linked to at least one regulatory sequence allowing for expression of said nucleic acid sequence encoding an antibody or Fc-fusion protein.

The present invention describes a H4-II-E rat hepatoma cell or derivatives or progenies thereof genetically modified by introducing a nucleic acid sequence/a gene of interest encoding a glycoprotein, preferably an antibody or Fc fusion protein operatively linked to at least one regulatory sequence allowing for expression of said nucleic acid sequence/gene of interest encoding an antibody or Fc-fusion protein.

In a specific embodiment said rat hepatoma cell or said H4-II-E cell is a cell derived from the European Collection of Cell Cultures (ECACC, Cat. no. 87031301) or from the American Type Culture Collection (ATCC, deposit no. CRL-1548) or said cell is a cell which is deposited with the European Collection of Cell Cultures under the number ECACC, Cat. no. 87031301 or is a derivative or progeny thereof or whereby said cell is deposited with the American Type Culture Collection ATCC under the deposit no. CRL-1548 or is a derivative or progeny thereof. In a further specific embodiment said rat hepatoma cell or said H4-II-E cell is a cell having the ECACC, Cat. no. 87031301 or the ATCC no. CRL-1548. In another specific embodiment said rat hepatoma cell is:

a) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H411 cell line (HPACC Nr. 89042702), H4-TG cell line (CRL-1578), H5 cell line (HPACC, Nr. 94101905) and H4-S cell line (HPACC Nr. 89102001), or
b) a cell which is deposited with the European Collection of Cell Cultures under the number ECACC, Cat. no. 87031301 or the American Type Culture Collection ATCC under the deposit no. CRL-1548 or
c) a cell which is deposited with the DSMZ (Deutsche Sammlung von Mikroorganismen und Zellkulturen GmbH) under the accession number DSM ACC3129 (H4-II-E), or
d) a cell which is deposited with the DSMZ (Deutsche Sammlung von Mikroorganismen und Zellkulturen GmbH) under the accession number DSM ACC3130 (H4-II-Es), or
e) a derivative or progeny of any one cell of a) or b) or c) or d). In a specific embodiment said rat hepatoma or said H4-II-E cell is a cell deposited with the DSMZ under the accession number DSM ACC3129 (H4-II-E) or DSM ACC3130 (H4-II-Es).

In another specific embodiment said rat hepatoma cell or said H4-II-E rat hepatoma cell has low fucosylation activity for adding fucose to glycosidic structures such as biantennary glycans, e.g. N-acetylglucosamine.

Specifically, the rat hepatoma cell or the H4-II-E rat hepatoma cell according to the invention further characterized in that i) the degree (or fraction) of glycosidic structures contained in the antibody or Fc-fusion protein expressed by said cell, which contain fucose, is less than 20%, 10% or 5% (of all glycans/glycosidic structures) or ii) the degree of glycosidic structures contained in said antibody or Fc-fusion protein expressed by said cell, which contain fucose, ranges between 0% to 20%, 0% to 10%, 0% to 5%, 0.5% to 20%, 0.5% to 10%, 0.5% to 5%, 1% to 20%, 1% to 10% or 1% to 5% (of all glycans/glycosidic structures). Specifically, the rat hepatoma cell or the H4-II-E rat hepatoma cell according to the invention further characterized in that less than 20% of glycans/glycosidic structures of said antibody or Fc-fusion protein contain fucose bound to the terminal reducing N-acetylglucosamine (GlcNAc) residue. Preferably said defucosylated glycosidic structures/glycans are N-linked, most preferably said glycosidic structures/glycans are attached by N-linked glycosylation at amino acid residue 297 according to the Kabat EU nomenclature for IgG1, IgG2, IgG3 and IgG4 antibodies (Kabat et al., 1991).

In a specific embodiment said rat hepatoma cell or said H4-II-E rat hepatoma cell has high galactosylation activity for adding galactose to glycosidic structures such as biantennary glycans, e.g. N-acetylglucosamine.

Specifically, the rat hepatoma cell or the H4-II-E rat hepatoma cell according to the invention further characterized in that i) the degree of glycans/glycosidic structures, preferably of the complex type, contained in said antibody or Fc-fusion protein expressed by said cell, which contain at least one, preferably one or two or one or more galactose residues, is more than 40%, 45% or 50% (of all glycans/glycosidic structures of the complex type) or ii) the degree of glycans/glycosidic structures, preferably of the complex type, contained in said antibody or Fc-fusion protein expressed by said cell, which contain at least one, preferably one or two or one or more galactose residues ranges between 40% to 100%, 45% to 100%, 50% to 100%, 51% to 100%, 40% to 99.5%, 45% to 99.5%, 50% to 99.5% or 51% to 99.5%, 40% to 99%, 45% to 99%, 50% to 99% or 51% to 99% (of all glycans/glycosidic structures of the complex type).

Preferably said galactosylated glycosidic structures/glycans contain one or two galactose residues (G1 or G2), preferably linked to N-acetylglucosamine (GlcNAc) at the terminal non-reducing end of said glycosidic structures. Preferably said glycosidic structures/glycans are N-linked at amino acid residue 297 according to the Kabat EU nomenclature for IgG1, IgG2, IgG3 and IgG4 antibodies (Kabat et al., 1991).

In a specific embodiment of the present invention the glycan/glycosidic structures are either G1 or G2. The glycan/glycosidic structures are preferably not G0.

In a specific embodiment said rat hepatoma cell or said H4-II-E rat hepatoma cell has high sialylation activity for adding sialic acid or neuraminic acid residues to glycosidic structures such as galactosylated biantennary glycans.

Specifically, the rat hepatoma cell or the H4-II-E rat hepatoma cell according to the invention further characterized in that i) the degree of (galactosylated) glycosidic structures contained in said antibody or Fc-fusion protein expressed by said cell, which contain terminal sialic acid or neuraminic acid residues, is more than 5% or more than 10% or ii) the degree of (galactosylated) glycosidic structures contained in said antibody or Fc-fusion protein expressed by said cell, which contain terminal sialic acid or neuraminic acid residues, ranges between 0-8%, 1-8%, 5-10%, 10-50% or 10-45%.

Preferably said glycosidic structures/glycans, which contain terminal sialic acid or neuraminic acid residues, are N-linked, most preferably they are attached by N-linked glycosylation at amino acid residue 297 according to the Kabat EU nomenclature for IgG1, IgG2, IgG3 and IgG4 antibodies (Kabat et al., 1991).

In a specific embodiment the rat hepatoma cell or the H4-II-E rat hepatoma cell according to the invention is isolated.

In a further specific embodiment the rat hepatoma cell or the H4-II-E rat hepatoma cell according to the invention is further characterized by carrying a selection marker gene such as neomycin-phosphotransferase (NPT), resistance genes against puromycin, hygromycin or zeocin or an amplifyable selection marker gene such as dihydrofolate reductase (DHFR) or glutamine synthetase (GS). In a specific embodiment, said NPT is the wild type neomycin-phosphotransferase.

In another specific embodiment of the rat hepatoma cell or the H4-II-E rat hepatoma cell according to the invention said regulatory sequence allowing for expression of said nucleic acid sequence encoding an antibody or Fc-fusion protein is a) a promoter or b) an enhancer or c) a 5′-UTR sequence, or d) a transcription enhancing (TE) element.

In a further embodiment of the invention said antibody or Fc fusion protein contains a glycosidic structure comprising the following sugar chain:

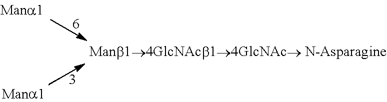

In another embodiment of the invention said antibody or Fc fusion protein contains a glycosidic structure linked to an N-Asparagine (N-Asn) residue, wherein said glycosidic structure comprises the following sugar chain:

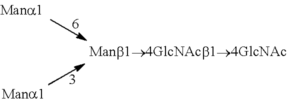

Specifically, the glycosidic structure comprises the following sugar chain:

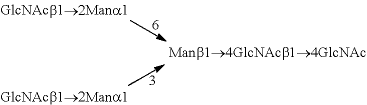

Preferably, said N-Asn in the embodiments described above is N-Asn (297) according to the Kabat EU nomenclature (Kabat et al., 1991).

In a specific embodiment less than 20%, preferably less than 10% or less than 5% of the GlcNAc residues at the reducing end of the glycan have fucose bound, more preferably no fucose is bound to the GlcNAc residue at the reducing end of the glycan as depicted by the following structure:

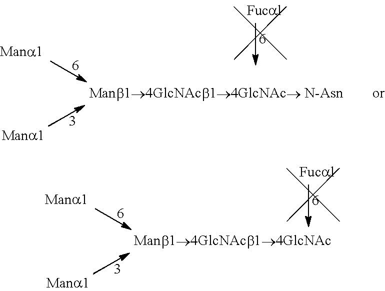

In another specific embodiment the rat hepatoma cell or the H4-II-E rat hepatoma cell according to the invention is adapted to growth in serum-free and calcium-reduced or preferably calcium-free medium.

In a further specific embodiment the rat hepatoma cell or the H4-II-E rat hepatoma cell according to the invention is adapted to growth in suspension culture. In another specific embodiment the rat hepatoma cell or the H4-II-E rat hepatoma cell according to the invention is additionally adapted to growth in medium which is free of any protein/peptide of animal origin. In another specific embodiment the rat hepatoma cell or the H4-II-E rat hepatoma cell according to the invention has low sensitivity to apoptosis and/or high robustness towards cellular stresses in comparison to YB2/0 cells.

The invention further describes a method for producing a glycoprotein of interest characterized by the following steps:

a) providing a rat hepatoma cell,
b) optionally adapting said cell of step a) to growth in suspension culture,
c) optionally adapting said cell of step a) and/or step b) to growth in serum-free medium,
d) optionally adapting said cell of step a) and/or step b) and/or step c) to growth in calcium-reduced or calcium-free medium,
e) transfecting this optionally adapted rat hepatoma cell with a nucleic acid sequence encoding a recombinant glycoprotein of interest,
f) cultivating said transfected cell of step e) under conditions which allow expression of said glycoprotein of interest,
g) optionally isolating and purifying said (glyco)protein of interest.

In a further embodiment said rat hepatoma cell is an H4-II-E cell, preferably said cell is:

i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548 or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

Preferably said cell is a cell having the ECACC, Cat. no. 87031301 or the ATCC no. CRL-1548. In a specifically preferred embodiment said rat hepatoma cell or said H4-II-E cell is a cell deposited with the DSMZ under the accession number DSM ACC3129 (H4-II-E) or DSM ACC3130 (H4-II-Es).

In a specific embodiment said medium of step b), c) or d) is additionally free of any protein/peptide of animal origin.

In a further specific embodiment said method is further characterized in that the transfection step e) comprises introducing an expression vector comprising a nucleic acid sequence encoding for said glycoprotein of interest operatively linked to at least one regulatory sequence allowing for expression of said nucleic acid sequence encoding a glycoprotein of interest into said rat hepatoma cell.

In another specific embodiment said method for producing a glycoprotein of interest is further characterized in that the cultivation step f) comprises adapting said transfected cell to growth in suspension culture, or to growth in serum-free medium, or to growth in calcium-reduced or calcium-free medium, or to growth in suspension culture in serum-free and calcium-reduced/calcium-free medium.

In a further specific embodiment the method according to the invention is further characterized in that said glycoprotein of interest is an antibody or Fc-fusion protein, preferably an antibody or Fc-fusion protein having

a) FcγRIIIa binding activity and preferably ADCC, or
b) complement binding activity and preferably CDC, or
c) binding activity to the neonatal Fc receptor FcRn and preferably serum stability, specifically long half life.

The invention further describes a method for producing a (recombinant) antibody or Fc fusion protein having

a) FcγRIIIa binding activity and/or
b) complement binding activity and/or
c) binding activity of the neonatal Fc receptor FcRn, comprising producing said antibody or Fc fusion protein in a rat hepatoma cell, whereby said rat hepatoma cell is preferably an H4-II-E cell, more preferably said cell is:
i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548 or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

Preferably said cell is a cell having the ECACC, Cat. no. 87031301 or the ATCC no. CRL-1548. In a specifically preferred embodiment said rat hepatoma cell or said H4-II-E cell is a cell deposited with the DSMZ under the accession number DSM ACC3129 (H4-II-E) or DSM ACC3130 (H4-II-Es).

Preferably, the (recombinant) antibody or Fc fusion protein is encoded by a nucleic acid sequence which is operatively linked to at least one regulatory sequence allowing for expression of said nucleic acid sequence encoding an antibody or Fc-fusion protein.

### IN A SPECIFIC EMBODIMENT

i) said antibody or Fc fusion protein of previous step a) has (increased) antibody dependent cellular cytotoxicity (ADCC) or
ii) said antibody or Fc fusion protein of previous step b) has (increased) complement dependent cytotoxicity (CDC) or
iii) said antibody or Fc fusion protein of previous step c) has serum stability. Specifically, iii) means that terminal sialylation of the glycosidic structures produced in H4-II-E cells, has a positive effect on the serum stability and catabolic half-life of therapeutic antibodies or Fc-fusion proteins. Thus, said antibodies or Fc fusion proteins of iii) have an increased half life/an increased serum stability/an increased catabolic half-life in comparison to antibodies or Fc fusion proteins produced in other cells, for example in CHO cells.

The increase in ADCC, CDC activity or the increase in half life can be measured by comparing the respective activity or half life of the antibody or Fc fusion protein produced in rat hepatoma cells or H4-II-E rat hepatoma cells with the activity of a corresponding antibody or Fc fusion protein produced in CHO cells, specifically in CHO DG44 cells.

The invention furthermore relates to a method for producing an antibody or Fc fusion protein having a promoted ADCC comprising introducing a DNA encoding said antibody in a rat hepatoma or a H4-II-E rat hepatoma cell, furthermore comprising cultivating and producing said antibody in said cell.

The invention furthermore relates to a method for producing an antibody or Fc fusion protein having a promoted CDC comprising introducing a DNA encoding said antibody in a rat hepatoma cell or a H4-II-E rat hepatoma cell, the method furthermore comprising cultivating and producing said antibody in said cell.

The invention further relates to a method for producing an antibody or Fc fusion protein having (promoted/increased) serum stability and half-life (especially if compared to antibody or Fc fusion protein produced in other cells, for example CHO cells) comprising introducing a DNA encoding said antibody in a H4-II-E rat hepatoma cell, the method further comprising cultivating and producing said antibody in said cell.

Producing specifically means cultivating the rat hepatoma cell under conditions which allow the expression of the antibody or Fc fusion protein and optionally purifying and isolating it, whereby said rat hepatoma cell is preferably an H4-II-E cell, more preferably said cell is:

i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548 or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

Preferably said cell is a cell having the ECACC, Cat. no. 87031301 or the ATCC no. CRL-1548. In a specifically preferred embodiment said rat hepatoma cell or said H4-II-E cell is a cell deposited with the DSMZ under the accession number DSM ACC3129 (H4-II-E) or DSM ACC3130 (H4-II-Es).

In a specific embodiment of the (production) methods according to the invention the glycoprotein of interest is further characterized in that i) the degree (or fraction) of glycosidic structures contained in the glycoprotein of interest, e.g. the antibody or Fc-fusion protein, which contain fucose, is less than 20%, less than 10% or less than 5% (of all glycans/glycosidic structures) or ii) the degree of glycosidic structures contained in said glycoprotein of interest, e.g. said antibody or Fc-fusion protein, which contain fucose, ranges between 0% to 20%, 0% to 10%, 0% to 5%, 0.5% to 20%, 0.5% to 10%, 0.5% to 5%, 1% to 20%, 1% to 10% or 1% to 5% (of all glycans/glycosidic structures). Specifically, the glycoprotein of interest, e.g. the antibody or Fc-fusion protein is further characterized in that less than 20% of glycans/glycosidic structures of said glycoprotein of interest, e.g. said antibody or Fc-fusion protein, contain fucose bound to the terminal reducing N-acetylglucosamine (GlcNAc) residue. Preferably said defucosylated glycosidic structures/glycans are N-linked, most preferably said glycosidic structures/glycans are attached by N-linked glycosylation at amino acid residue 297 according to the Kabat EU nomenclature for IgG1, IgG2, IgG3 and IgG4 antibodies (Kabat et al., 1991).

In another specific embodiment of the (production) methods according to the invention the glycoprotein of interest is further characterized in that i) the degree of glycans/glycosidic structures, preferably of the complex type, contained in said glycoprotein of interest, e.g. said antibody or Fc-fusion protein, which contain at least one, preferably one or two or one or more galactose residues, is more than 40%, 45% or 50% (of all glycans/glycosidic structures of the complex type) or ii) the degree of glycans/glycosidic structures, preferably of the complex type, contained in said glycoprotein of interest, e.g. said antibody or Fc-fusion protein, which contain at least one, preferably one or two or one or more galactose residues ranges between 40% to 100%, 45% to 100%, 50% to 100%, 51% to 100%, 40% to 99.5%, 45% to 99.5%, 50% to 99.5% or 51% to 99.5%, 40% to 99%, 45% to 99%, 50% to 99% or 51% to 99% (of all glycans/glycosidic structures of the complex type).

Preferably said galactosylated glycosidic structures/glycans contain one or two galactose residues (G1 or G2), preferably linked to N-acetylglucosamine (GlcNAc) at the terminal non-reducing end of said glycosidic structures. Preferably said glycosidic structures/glycans are N-linked at amino acid residue 297 according to the Kabat EU nomenclature for IgG1, IgG2, IgG3 and IgG4 antibodies (Kabat et al., 1991). In a specific embodiment of the present invention the glycan/glycosidic structures are either G1 or G2. The glycan/glycosidic structures are preferably not G0.

In a further specific embodiment of the (production) methods according to the invention the glycoprotein of interest is further characterized in that i) the degree of (galactosylated) glycosidic structures contained in said glycoprotein, e.g. said antibody or Fc-fusion protein, which contain terminal sialic acid or neuraminic acid residues, is more than 5% or more than 10% or ii) the degree of (galactosylated) glycosidic structures contained in said glycoprotein of interest, e.g. said antibody or Fc-fusion protein, which contain terminal sialic acid or neuraminic acid residues, ranges between 0-8%, 1-8%, 5-10%, 10-50% or 10-45%. Preferably said glycosidic structures/glycans, which contain terminal sialic acid or neuraminic acid residues, are N-linked, most preferably they are attached by N-linked glycosylation at amino acid residue 297 according to the Kabat EU nomenclature for IgG1, IgG2, IgG3 and IgG4 antibodies (Kabat et al., 1991).

The invention further describes a method of generating a (host) cell for production of recombinant glycoprotein comprising:

a) providing a rat hepatoma cell,
b) adapting said rat hepatoma cell of step a) to growth in suspension culture, and
c) adapting said rat hepatoma cell of step a) to growth in serum-free medium, and
d) adapting said rat hepatoma cell of step a) to growth in calcium-reduced or calcium-free medium, and
e) optionally adapting said rat hepatoma cell of step a) to growth in medium free of any protein/peptide of animal origin, and
f) optionally selecting a single cell clone,
g) obtaining a (production) (host) cell. Preferably said rat hepatoma cell is an H4-II-E cell, more preferably said cell is:
i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548 or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

Preferably said cell is a cell having the ECACC, Cat. no. 87031301 or the ATCC no. CRL-1548. In a specifically preferred embodiment said rat hepatoma or said H4-II-E cell is a cell deposited with the DSMZ under the accession number DSM ACC3129 (H4-II-E) or DSM ACC3130 (H4-II-Es).

The invention further describes a method of generating a (host) cell for production of recombinant glycoprotein comprising:

a) providing a H4-II-E rat hepatoma cell,
b) adapting said H4-II-E rat hepatoma cell of step a) to growth in suspension culture, and
c) adapting said H4-II-E rat hepatoma cell of step a) to growth in serum-free medium, and
d) adapting said H4-II-E rat hepatoma cell of step a) to growth in calcium-reduced or calcium-free medium, and
e) optionally adapting said H4-II-E rat hepatoma cell of step a) to growth in medium free of any protein/peptide of animal origin, and
f) optionally selecting a single cell clone,
g) obtaining a (host) cell.

In a specific embodiment said method further comprises:

h) transfecting said obtained H4-II-E rat hepatoma host cell of step g) with a nucleic acid sequence encoding a glycoprotein of interest, and
i) optionally cultivating said transfected cell of step h) under conditions which allow expression of said glycoprotein of interest.

Preferably, said glycoprotein of interest is an antibody or Fc fusion protein, most preferably an antibody or Fc fusion protein having ADCC and/or CDC and/or serum stability, and/or specifically long half-life.

Preferably, the antibody is a human antibody, a humanized antibody, a human chimeric antibody or a human CDR-grafted antibody.

The invention further relates to a (host) cell generated according to said method of generating a host cell for production of recombinant glycoprotein as described above.

The invention further relates to a (rat hepatoma) cell deposited with the DSMZ (Deutsche Sammlung von Mikroorganismen and Zellkulturen GmbH) under the accession number DSM ACC3130 (H4-II-Es).

The invention further relates to a (rat hepatoma) cell deposited with the DSMZ (Deutsche Sammlung von Mikroorganismen und Zellkulturen GmbH) under the accession number DSM ACC3129 (H4-II-E).

The invention furthermore relates to the use of a rat hepatoma cell as a host cell for biopharmaceutical production or as a host cell for the production of (recombinant) glycoprotein, preferably antibody or Fc fusion protein. Preferably said rat hepatoma cell is an H4-II-E cell, more preferably said cell is:

i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548 or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

Preferably said cell is a cell having the ECACC, Cat. no. 87031301 or the ATCC no. CRL-1548. In a specifically preferred embodiment said rat hepatoma or said H4-II-E cell is a cell deposited with the DSMZ under the accession number DSM ACC3129 (H4-II-E) or DSM ACC3130 (H4-II-Es).

The practice of the present invention will employ, unless otherwise indicated, conventional techniques of cell biology, molecular biology, cell culture, immunology and the like which are in the skill of one in the art. These techniques are fully disclosed in the current literature. All publications and patent applications mentioned in this specification are indicative of the level of skill of those skilled in the art to which this invention pertains. All publications and patent applications cited herein are hereby incorporated by reference in their entirety in order to more fully describe the state of the art to which this invention pertains. The invention generally described above will be more readily understood by reference to the following example, which is hereby included merely for the purpose of illustration of certain embodiments of the present invention and is not intended to limit the invention in any way.

### EXPERIMENTAL

### Materials and Methods

### DNA Constructs

The plasmid DNA used for the stable transfection of different cell lines encodes an IgG1 antibody heavy and light chain under the control of viral or ubiquitous promoters, as well as the selection markers neo and dhfr.

### Cell Lines and Cell Cultivation

The dihydrofolate reductase-deficient CHO cell line CHO/DG44 (Urlaub and Chasin, 1980) and the variant CHO cell line deficient in endogenous GMD, Pro-Lec13.6A (Ripka et al., 1986) have been described previously. The rat hybridoma cell line YB2/0, as well as the rat hepatoma cell line H4-II-E are derived from the European Collection of Cell Cultures (ECACC, Cat. no 85110501, and 87031301).

The H4-II-E cell line of the present invention has been deposited under the Budapest treaty with the Deutsche Sammlung von Mikroorganismen und Zellkulturen GmbH (DSMZ), Inhoffenstrasse 7B, D-38124 Braunschweig, Germany under the accession number DSM ACC3129 (H4-II-E) on 28 Jun. 2011. H4-II-E cells, such as the cells deposited with the DSMZ under the accession number DSM ACC3129, are cultured in MEMalpha (Invitrogen) or EMEM containing 5% FCS. Adherent cells are trypsinized with Trypsin/EDTA (Invitrogen) every 3-4 days and seeded in fresh medium at cell density of approximately 20.000-30.000 cell/cm² in tissue culture treated plates or T-flasks (Nunc, Denmark).

The H4-II-E cell line adapted to the growth in suspension in serum-free, Ca2+-free medium described in the present invention has been deposited under the Budapest treaty with the Deutsche Sammlung von Mikroorganismen und Zellkulturen GmbH (DSMZ), Inhoffenstrasse 7B, D-38124 Braunschweig, Germany under the accession number DSM ACC3130 (H4-II-Es) on 28 Jun. 2011. H4-II-E cells adapted to the growth in suspension in serum-free, Ca2+-free medium, such as the cells deposited with the DSMZ under the accession number DSM ACC3130, are cultured in shake flasks (Corning) at 100-120 rpm at a temperature of 37° C. and in an atmosphere containing 5% CO₂. H4-II-E suspension cultures are subcultivated every 3-4 days with seeding densities of 300,000-400,000 cells/mL. CHO-DG44 cells as well as adapted CHO-Lec13 cells, YB2/0 cells and H4-II-E cells are cultivated in suspension in serum-free media in incubators (Thermo, Germany) at a temperature of 37° C. and in an atmosphere containing 5% CO₂ in surface-aerated T-flasks (Nunc, Denmark) or shake flasks (Corning) at 100-120 rpm. Suspension cultures are subcultivated every 2-3 days with seeding densities of 200,000-300,000 cells/mL. The cell concentration is determined in all cultures by using a hemocytometer. Viability is assessed by the trypan blue exclusion method. All suspension cultures are cultured in BI-proprietary media or MEM Joklik Modification (Sigma) or MEM Spinner Modification (Sigma).

### Predictive Analysis for Glycosylation Properties

Different cell lines are selected and analysed for the expression level of enzymes involved in the glycosylation machinery. These cell lines are further analysed for the binding of lectins (carbohydrate specific binding proteins) to the glycoprotein containing cell surface. Values obtained for each cell line are normalized to the values obtained for CHO, which is a well known production system for recombinant proteins and displays well characterized glycosylation patterns. The presence of certain glycostructures on the cell surface is an indicator for the glycosylation capacity of the cell.

### Transfection and Isolation of Stably Transfected Cell Populations

Cell lines are transfected with Lipofectamine™ and PLUS™ Reagents (both Invitrogen, Germany) or by Nucleofection (Amaxa/Lonza) according to the guidelines provided by the manufacturer.

To isolate stably transfected, IgG1 producing cell populations, cells are transfected and plated in the presence of the selective antibiotics G418 and MTX after 48 hours. Surviving cell populations are isolated after approximately 3 weeks of selection and are analysed for their productivity by ELISA using samples from the cell culture supernatant.

### Serum-Free Fed-Batch Culture

H4-II-E production cells are adapted to the growth in suspension and in serum-free media. For a serum-free Fed-Batch cultivation, adapted cells are seeded at 400.000 cells/ml or 600.000 cells/ml in BI-proprietary production medium or MEM Spinner Modification (Sigma) and cultures are agitated at 120 rpm in 37° C. and 5% CO₂. Culture parameteres including pH, glucose and lactate concentrations are determined daily. The pH is adjusted to pH 7.0 using NaCO₃ as needed, glucose is feeded to maintain a glucose content of approximately 4 g/L. Cell densities and viability are determined by trypan-blue exclusion using an automated CEDEX cell quantification system (Innovatis). The antibody or Fc-fusion protein concentration in culture supernatant is measured by enzyme-linked immunosorbent assay (ELISA) specific for human IgG as described.

Prior to the suspension adaptation, adherent H4-II-E production clones can be used to produce material in stationary Fed-Batch cultures in serum-free medium. H4-II-E IgG1-producing cells are grown to confluence in Hyperflasks (Corning) in MEMalpha containing 5% FCS. The culture medium is then replaced with serum-free BI-proprietary medium or serum-free commercial medium e.g. HyClone SFM4 (Thermo Fisher Scientific). After culturing for 13 days, antibody is purified from the culture media using MabSelect (Amersham) and stored in 10 mM citrate/0.15M NaCl (pH 6.0).

CHO-DG44, CHO-Lec13 and YB2/0 IgG1 producing cells are seeded at 300.000 cells/ml in BI-proprietary production medium and cultures are agitated at 120 rpm in 37° C. and 5% CO₂ which is later reduced to 2% as cell numbers increase. Culture parameteres including pH, glucose and lactate concentrations are determined daily. The pH is adjusted to pH 7.0 using

NaCO₃ as needed, glucose is feeded to maintain a glucose content of approximately 4 g/L. BI-proprietary feed solution is added every 24 hrs. Cell densities and viability are determined by trypan-blue exclusion using an automated CEDEX cell quantification system (Innovatis). The antibody concentration in culture supernatant is measured by enzyme-linked immunosorbent assay (ELISA) specific for human IgG as described.

### ELISA

Quantification of IgG molecules in the supernatant of the cell cultures is performed via sandwich ELISA technology. ELISA plates are coated using a goat anti-human IgG Fc-Fragment antibody (Dianova, Germany) at 4° C. over night. After washing and blocking of the plates with 1% BSA solution, the samples are added and incubated for 1.5 hours. After washing, the detection antibody (alkaline-phosphatase conjugated goat anti-human kappa light chain antibody) is added and colorimetric detection is performed by incubation with 4-nitrophenyl phosphate disodium salt hexahydrate (Sigma, Germany) as substrate. After 20 min incubation in the dark, the reaction is stopped and the absorbance is immediately measured using an absorbance reader (Tecan, Germany) with 405/492 nm. The concentration calculated according to the standard curve which is present on each plate.

Purification of IgG1 from the Cell Culture Supernatant Recombinant antibodies are purified from the serumfree culture supernatant by Protein A-affinity chromatography using MabSelect™ (Amersham Biosciences) and stored in 10 mM citrate/0.15M NaCl (pH 6.0). The concentration of the purified antibodies is measured by Protein A-HPLC.

### Analysis of the Glycosylation Pattern

To elucidate the structure and composition of the Fc-glycosylation of IgGs produced in different cell lines, the glycans are released from the purified antibody after reduction by enzymatic digestion with PNGase F. Glycans are purified, fluorescently labelled with 2-Aminobenzamide (2-AB) and fractionated on a HPLC column before and after treatment with exoglycosidic enzymes (e.g. α-mannosidase, neuraminidase, β-galactosidase, α-galactosidase, β-hexosaminidase, and α-fucosidase). The percentages of fucosylated vs. non-fucosylated biantennary glycans, and other glycosidic structures are calculated from the chromatographic peak area ratios before and after exoglycosidic digestion and allow the qualitative and quantitative verification of the glycostructures and compostition.

FcγRIIIa-Binding Assay

The binding kinetics of IgG1 produced in different cell lines to FcgRIIIa is measured using a BIAcore T100 instrument and CM5 sensor chips (BIACORE, Uppsala, Sweden) as follows. Soluble recombinant FcgRIIIa is immobilized onto the BIAcore sensor chip. The purified IgG1s are diluted in HBS-EP buffer (0.01 M HEPES, 0.15 M NaCl, 3 mM EDTA, 0.005% Surfactant P20, pH 7.4) at six different concentrations (from 4.17 to 133.3 nM) and each diluted IgG1 is injected over the receptor-captured sensor surface at a flow rate of 5 mL/min The experiments are performed at 25° C. with HBS-EP as the running buffer. Buffer solution without sample IgG1 is injected over the receptor-captured sensor surface as a blank control. Soluble FcgRIIIa and IgG1 bound to the sensor surface are removed by injecting 7.5 mM HCl at a flow rate of 10 mL/min for 30 s. The data obtained by the injection of IgG1 are corrected for the blank control prior to data analysis. An affinity (KD) for FcgRIIIa is calculated by steady-state analysis using BIAcore T100 kinetic evaluation software (BIACORE).

### FcγRIIb-Binding Assay

The binding kinetics of IgG1 produced in different cell lines to FcgRIIb is measured using a BIAcore T100 instrument and CM5 sensor chips (BIACORE, Uppsala, Sweden) as follows. Soluble recombinant FcgRIIIa is immobilized onto the BIAcore sensor chip. The purified IgG1s are diluted in HBS-EP buffer (0.01 M HEPES, 0.15 M NaCl, 3 mM EDTA, 0.005% Surfactant P20, pH 7.4) at six different concentrations (from 4.17 to 133.3 nM) and each diluted IgG1 is injected over the receptor-captured sensor surface at a flow rate of 5 mL/min The experiments are performed at 25° C. with HBS-EP as the running buffer. Buffer solution without sample IgG1 is injected over the receptor-captured sensor surface as a blank control. Soluble FcgRIIb and IgG1 bound to the sensor surface are removed by injecting 7.5 mM HCl at a flow rate of 10 mL/min for 30 s. The data obtained by the injection of IgG1 are corrected for the blank control prior to data analysis. An affinity (KD) for FcgRIIb is calculated by steady-state analysis using BIAcore T100 kinetic evaluation software (BIACORE).

### ADCC Assay

ADCC assays are performed by the lactate dehydrogenase (LDH) release assay using as effector cells human peripheral blood mononuclear cells (PBMC) prepared from healthy donors by Lymphoprep (AXIS SHIELD, Dundee, UK). Aliquots of target tumor cells, the human Burkitt's lymphoma cell line Ramos, expressing human CD20, or HER2-positive breast cancer cell lines are distributed into 96-well U-bottomed plates (10.000 cells in 50 μl/well) and incubated with serial dilutions of antibodies (50 μL) in the presence of the PBMC (100 μL) at an E/T ratio of 20/1. After incubation for 4 h at 37° C., the supernatant LDH activity is measured using a Non-Radioactive Cytotoxicity Assay Kit (Promega, Madison, Wis.). The percent specific cytolysis is calculated from the sample activities according to the formula: specific lysis [%]=100*(E−S_{E}−S_{T})/(M−S_{T}), where E is the experimental release (activity in the supernatant from target cells incubated with antibody and effector cells), S_{E} is the spontaneous release in the presence of effector cells (activity in the supernatant from effector cells with medium alone), S_{T} is the spontaneous release of target cells (activity in the supernatant from target cells incubated with medium alone), and M is the maximum release of target cells (activity released from target cells lysed with 9% Triton X-100).

C1q-Binding Assay

The ability of each purified IgG to bind to the C1 q component of the complement is studied by a flow cytometric assay using purified human complement C1q. Human Burkitt's lymphoma cell line Ramos, expressing human CD20, or HER2-positive breast cancer cell lines are adjusted to 2*10⁶ cells/mL and incubated with serial dilutions of anti-human CD20 IgG or anti-human HER2 for 30 min in PBS containing 1% (w/v) BSA. After washing with PBS containing 1% (w/v) BSA, purified human complement C1q (Biogenesis Ltd, Poole, UK) is added at a final concentration of 20 mg/mL and bound to the cell-bound IgGs at 37° C. for 30 min Cells are then washed and incubated with fluorescein isothiocyanate-conjugated polyclonal antibodies against human C1q (Acris Antibodies GmbH, Hiddenhausen, Germany) for 30 min Stained cells are analyzed by flow cytometry using FACSCalibur.

### CDC Assay

CDC activity is determined by the LDH assay. Briefly, the target human Burkitt's lymphoma cell line Ramos, expressing human CD20, or HER2-positive breast cancer cell lines, 2-fold diluted human serum complement (Sigma-Aldrich), and serial dilutions of anti-human CD20or anti-human HER2-IgG1 are incubated in 96-well flat-bottomed plates (Greiner) for 3 h at 37° C. Cell proliferation LDH reagent (Roche Diagnostics, Basel, Switzerland) is added to the wells (15 μL/well) and incubated for 30 min at 37° C. Absorbance in the wells is measured at 492 nm using a microplate reader (Tecan, Germany) and expressed in relative absorbance units (RAU) as an index of the viable cell number. The percent CDC is calculated according to the formula: CDC activity [%]=100*(RAU_{background}−RAUₜₑₛₜ)/RAU_{background}.

### FcRn-Binding Assay

A recombinant soluble human FcRn-b2 microglobulin complex is expressed in CHO/DG44 cells and purified from the culture supernatant by Ni-NTA chromatography (Qiagen). The kinetics of the human IgG1-FcRn interaction is measured using a BIAcore T100 instrument and CM5 sensor chips. Antihuman b2-microglobulin monoclonal antibody (Abcam, Cambridge, UK) is immobilized onto the chip using an amine-coupling kit (BIACORE). Soluble FcRn-b2 microglobulin complex is captured by the immobilized anti-b2-microglobulin antibody by injecting the complex at a flow rate of 5 mL/min Buffer solution without the complex is injected over the antibody-captured sensor surface as a blank control. Each purified IgG is diluted in HBS-EP+ buffer (0.01 M HEPES, 0.15M NaCl, 3 mM EDTA, 0.05% Surfactant P20) whose pH is adjusted to 6.0 at five different concentrations (from 4.17 to 66.7 nM), and each diluted IgG1 is injected over the complex-captured sensor surface or blank at a flow rate of 5 mL/min Soluble FcRn and IgG1 bound to the sensor surface are removed by injecting 7.5 mM HCl at a flow rate of 60 mL/min for 1 min. The experiments are performed at 25° C. with HBS-EP+ as a running buffer. The data obtained by blank subtraction are used for the data analysis. An apparent association rate constant (ka), a dissociation rate constant (kd), and the binding affinity (KD) are calculated by the bivalent fitting model using BIAcore T100 evaluation software.

### Pharmacokinetic Analysis in Mice

For purified anti-human CD20 IgG1, produced in CHO or H4-II-E cells, three 13-week-old female ddY mice (Charles River Laboratories) are injected into the tail vein with 20 mg of the IgG1. Peripheral blood samples are taken from the tail vein at 0.083 (5 min), 0.5, 1, 6, 24, 60, 120, 216, 312, and 384 h, and the antibody concentration in the plasma is measured by an ELISA specific for human IgG1 as described previously. The serum half-life of the administrated IgG1 is calculated from the slope of the elimination beta-phase.

### EXAMPLES

### Example 1

### Prediction Shows Significant Differences Between Cell Lines in the Relative Content of Glycostructures on Secreted Proteins

Cell lines originating from different species and within these species being derived from different tissues or cell lineages, are selected and analysed for surface structures and enzymatic activities (FIG. 1). Based on the results of the analysis, each cell line can be assigned the capability of synthesizing certain glycosylation patterns. The results of the analysis show that cell lines are different in their glycosylation properties depending on both, the species and tissue or cellular lineage from which they originate. Only few cell lines do naturally show the capacity for low fucosylation. Some cell lines potentially generate sialylated structures and several cell lines form immunogenic glycostructures (Gal-1,3-Gal and NeuGc). Neither the species origin of a given cell line, nor the tissue, organ or cell lineage utterly determine the glycosylation capacity of a cell. Both, the species origin and the tissue cell lineage can influence the capability of a cell to synthesize certain glycosylation patterns. Yet, it is not possible to fully predict the glycosylation properties of a cell line solely based on the knowledge of the glycopattern derived in another cell line originating from the same tissue and/or species (FIG. 1).

The rat hepatoma cell line H4-II-E can be distinguished from all selected and analysed cell lines in its high potential to generate antibodies with advantageous glycoproperties, and is therefore chosen for further evaluation.

The cell line H4-II-E is the only one of all selected and analysed cell lines, which shows no detectable signs of fucosylation in this analysis. This is surprising, since neither other rat cell lines nor other liver cell lines do in the same way accumulate beneficial glycoproperties like reduced fucosylation, lack of immunogenic residues, or the presence of α-2,6 linked sialic acid (FIG. 1).

It is surprising that particularly the rat cell H4-II-E produces glycosylations with a low content of fucose, since antibodies from rat blood serum are heavily fucosylated, while intrinsic antibodies in other species like rabbit or cat have a low content of fucose.

In contrast to the fucose content, there might be a species dependent predisposition for the generation of potential immunogenic glycostructures like Gal-1,3-Gal and NeuGc in certain cell lines. None of the selected and analysed human or rat cell lines shows such residues, while in agreement with published data, cell lines derived from mouse, rabbit and other species, consistently produce such structures, potentially inducing immunogenic reactions in humans (Jenkins et al., 1996; Raju et al., 2000).

### Example 2

### Antibodies Produced in the Rat Hepatoma Cell Line H4-II-E Show a Significantly Reduced Fucosylation of Fc Glycans Compared to Other Known Production Cell Lines

To verify the predicted glycosylation properties produced in H4-II-E cells, stable IgG1 antibody producing H4-II-E cells are generated by transfection with DNA constructs encoding the light and heavy chain of an IgG1 antibody and subsequent antibiotic selection for resistance markers also present on the transfected DNA constructs. IgG1 producing cell populations are obtained and adapted to the growth in serum-free chemically defined medium. The IgG1 antibody produced in H4-II-E cells and purified from the cell culture supernatant by Protein A chromatography is intact, giving discrete bands for heavy and light chain after electrophoretical separation (data not shown). The structure and composition of Fc glycans on Protein A purified IgG1 antibodies produced in H4-II-E cells is analysed and compared to the glycosylation patterns derived on the same antibody produced in CHO-DG44, CHO-Lec13 mutants and YB2/0 rat myeloma cells. In all four IgG1 antibody preparations from the different host cell lines, biantennary glycans make up the largest proportion of all measured Fc glycans. Only for antibodies produced in YB2/0 cells, the proportion of other structures, which are mainly hybrid, uncomplete glycans or high-mannose structures also make up a significant part of approximately 23% (FIG. 2). Regarding the fraction of biantennary glycans, CHO-DG44 cells, as previously reported, produce largely (—95%) the fucosylated structures. Antibodies produced in H4-II-E cells, in contrast to the other cells, clearly carry a large proportion of non-fucosylated biantennary glycans. More than 80% of IgG1 expressed in H4-II-E cells contains non fucosylated biantennary glycans, which is a significantly higher proportion than in antibodies produced in the cell lines YB2/0 or the CHO mutant Lec13 (FIG. 2). YB2/0 and CHO-Lec13 cells also produced fucose-free glycans, yet, the percentual difference between fucose-free and fucose-containing form is not as dramatic as with H4-II-E produced antibodies (FIG. 2). IgG1 antibodies produced in H4-II-E cells, despite not being genetically engineered, clearly show the highest proportion of non-fucosylated biantennary glycans, thereby confirming the prediction after the initial selection and analysis for advantageous glycostructures (FIG. 1). With the known correlation of low fucosylation leading to enhanced ADCC activation, H4-II-E cells are superior to the other host cell lines in producing antibodies with high activity in FcγRIII dependent effector functions.

### Example 3

### Antibodies Produced in the Rat Hepatoma Cell Line H4-II-E Show a Significantly Increased Galactosylation of Fc Glycans Compared to CHO Produced Antibodies

The detailed analysis of the glycosylation pattern of IgG1 produced in H4-II-E cells reveals, that the content of galactosylated glycoforms is elevated compared to antibodies produced in CHO-DG44, the host cell line typically used for biopharmaceutical protein production (FIG. 3). In IgG1 antibodies produced in H4-II-E, >40% of the biantennary glycans are galactosylated, a ratio which is significantly higher than in antibodies which are expressed in CHO cells (FIG. 3). An increased galactosylation of biantennary glycans improves the potential of antibodies to activate the complement system. Together with the reduced fucosylation of Fc glycans produced in H4-II-E cells, this results in a higher efficiency of the H4-II-E produced antibodies in both types of antibody-dependent effector functions, the ADCC and the CDC. Galactosylation of biantennary glycans, besid improving the Fc-binding and activation of components of the complement system, is the basis for further modification of the glycans at the terminal position.

### Example 4

### Antibodies Produced in the Rat Hepatoma Cell Line H4-II-E Show Detectable Sialylation of Fc Glycans in Contrast to CHO Produced Antibodies

Sialic acid or neuraminic acid residues can be found α-2,3 or α-2,6 linked to the preceding galactose residues at the terminal end of the antennas of the complex type N-glycans. Concerning sialylation, the results obtained after analysing the glycostructures of an IgG1 antibody produced in H4-II-E cells are in agreement with the predicted glycostructures (FIG. 1). Approximately 8% of the antibodies produced in H4-II-E cells carry terminal sialic acid residues which can be cleaved by treatment of the released glycans with the exoglycosidase neuraminidase (FIG. 4). In contrast to antibodies produced in H4-II-E cells, and in agreement with the predicted glycosylation pattern, antibodies produced in CHO-DG44 cells do not carry terminal sialic acid residues (FIG. 4). Terminal sialic acid residues can have different effects on the activity and stability of the modified antibodies. Recent publications indicate that sialic acid can inhibit inflammatory activities of antibodies (Burton and Dwek, 2006; Scallon et al., 2007). Other data indicate that the absence of sialic acid leads to an enhanced metabolic rate in the liver of mice, indicating a clearance by liver based receptors (Wright et al., 2000). Derivatives of sialic acid like N-glycolylneuraminic acid (NeuGc) are found to be immunogenic in humans (Noguchi et al., 1995), however, evidence of NeuGc modifications on proteins produced in H4-II-E cells could not be detected (FIG. 1).

### Example 5

### H4-II-E Rat Hepatoma Cells Adapted to Growth in Suspension in Serum-Free Medium

H4-II-E cells cultured in serum containing media (EMEM(EBSS)+2 mM Glutamine+1% Non Essential Amino Acids+10% Foetal Bovine Serum (FBS)) grow as adherent cell layers. For the industrial production of biopharmaceutical proteins, H4-II-E cells are grown in serum-free medium, preferably in chemically defined, animal component free media, and as suspension cultures. To adapt H4-II-E cells to suspension culture in serum-free medium, cells are first expanded adherently and in serum-containing medium showing an average doubling time of 30 hours. Cells are then detached and dissociated by treatment with Trypsin/EDTA, harvested by centrifugation, extensively washed with Calcium and Magnesium-free Dulbecco's Phosphate Buffered Saline (DPBS) and suspended at densities of 200.000 cells/ml to 600.000 cells/ml in a serum-free BI proprietary medium which is specifically designed for the suspension culture of H4-II-E cells or in commercial AEM (Invitrogen) or PEM (Invitrogen) Media. The critical aspect for culturing of H4-II-E cells in suspension with population doubling times of 24 hours to 32 hours is the use of a calcium-free or calcium-reduced medium. The cell suspension is cultured in shake flasks (Corning) at 37° C., 100-120 rpm and 5% CO₂. Cells are passaged every 3-4 days and suspended in fresh medium. After several passages, H4-II-E cells grow constantly as a single cell suspension with a doubling time of 24 hours to 32 hours. H4-II-E cells grow well in different cultivation formats and can be cultured with high viabilities in suspension in FedBatch processes for more than 10 days. In a standard CHO optimized medium, H4-II-E cells reach a maximal density of 8.000.000 cells/ml which can be further improved using H4-II-E optimized medium (FIG. 5). Taken together, adapted H4-II-E cells are well-suited as a (production) host cell (line) for large scale biopharmaceutical protein production.

### Example 6

### The H4-II-E Cell has Low Sensitivity to Apoptosis and/or High Robustness Towards Cellular Stresses in Comparison to YB2/0 Cells

H4-II-E cells in contrast to YB2/0 cells show a high robustness towards cellular stress induced by high temperature, low or high osmolarity, pH-changes, mechanical stimulation, or treatment with chemicals or drugs. Equal cell numbers of H4-II-E rat hepatoma cells and YB2/0 rat myeloma cells in full culture medium (serum-free BI medium (Ca-free) for H4-II-E cells, RPMI+10% FCS for YB2/0 cells) are exposed to a dose of the following stressors (period of stress shown in brackets): +42° C. (for 2 hours, see FIG. 6A), exposure to low or high salt concentrations (for 24 hours, see FIG. 6B), exposure to 2 or 5 μg/ml Puromycin (for 48 hours, see FIG. 6C): After the temperature challenge, the cells are cultured in full medium at 37° C., 5% CO2, for 24 hours before analysing the cell number and viability by trypan blue exclusion staining. H4-II-E cells show a significantly higher robustness and survival after cellular stresses or apoptosis inducing stimuli than YB2/0 cells (FIG. 6). The low sensitivity and high robustness of H4-II-E cells towards cellular stressors, makes H4-II-E a superior system for biopharmaceutical large scale production processes, where high cell viability and survival are required over prolonged culture periods.

### Example 7

### Single Cell Suspension Cultivation of H4-II-E Cells Requires Ca²⁺-Reduced or Ca²⁺-Free, Serum Free Media

The critical aspect for culturing of H4-II-E cells in suspension with population doubling times of 24 hours to 32 hours is the use of a calcium-free or calcium-reduced medium. Two Calcium-reduced or Calcium-free media are identified by analysing the Ca-concentration with a Hitachi 917 (Roche) (FIG. 7A). Both, the Calcium-reduced medium AEM and the Ca-free BI medium allow the single cell suspension cultivation of H4-II-E cells (FIG. 7B, C). If AEM is supplemented with 1 mM CaCl₂ (FIG. 7C) or if cells are seeded in Ca-containing BI medium (FIG. 7E), the cells form large aggregates and single cell suspension growth is blocked. A reduction of free Calcium ions in media containing Calcium can also be achieved by the addition of EDTA to the medium, resulting in reduced aggregation of H4-II-E cells in such EDTA supplemented media (FIG. 7F).

### Example 8

### H4-II-E Cell Aggregation is Ca²⁺-Concentration Dependent, and Mg²⁺-Independent

To address the question if the effect of Calcium ions on H4-II-E cell aggregation can also be achieved by other divalent cations commonly present in cell culture media, suspension cultures of H4-II-E cells are subjected to increasing concentrations of CaCl₂ or MgCl₂ (FIG. 8). An increase in H4-II-E cell aggregation can already be observed if 50 μMol/L CaCl₂ is added to Ca-free BI medium. The aggregation rate and compactness of cell aggregates further increases with increasing concentrations of CaCl₂ added to the Ca-free medium (FIG. 8A). In contrast, the addition of equal concentrations of MgCl₂ to the medium has no visible effect on the single cell suspension or cell aggregation (FIG. 8B). In AEM medium, the addition of 250 μM/L CaCl₂ or of higher CaCl₂ concentrations significantly impairs the growth of H4-II-E cells already after 3 days in culture. In AEM medium supplemented with 250 μM/L CaCl₂ or of higher CaCl₂ concentrations, more than 80% of the cells are forming compact aggregates, which is unwanted in cell culture fermentation processes. In contrast to CaCl₂, increasing concentrations of MgCl₂ do not have an effect on the aggregation rate of H4-II-E cells.

### Example 9

### Anti-CD20 IgG1 Antibodies Produced IN H4-II-E Rat Hepatoma Cells Bind to the Fc Receptors CD16-V158 and CD16-F158 (FCGRIIIA) with Higher Affinity than Anti-CD20 IgG1 Produced in CHO

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

The binding kinetics of anti-CD20 IgG1 produced in H4-II-E cells and in CHO cells to FcγRIIIa receptors CD16-V158 and CD16-F158 is measured using a BIAcore assay. Anti-CD20 IgG1 produced in H4-II-E cells shows a significantly higher affinity to both variants of the FcγRIIIa than anti-CD20 IgG1 produced in CHO cells (FIG. 9).

### Example 10

### Anti-CD20 IgG4 Antibodies Produced in H4-II-E Rat Hepatoma Cells Bind to the Fc Receptors CD16-V158 and CD16-F158 (FCGRIIIA) With Higher Affinity than Anti-CD20 IgG4 Produced in CHO

An anti-CD20 IgG4 antibody expression vector is used to stably transfect CHO cells and H4-is II-E cells respectively. Anti-CD20 IgG4 antibody producing H4-II-E cells are generated by transfection with DNA constructs encoding the heavy chain (SEQ ID NO:4) and light chain (SEQ ID NO:5) of the anti-CD20 IgG4 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

The binding kinetics of anti-CD20 IgG4 produced in H4-II-E cells and in CHO cells to FcγRIIIa receptors CD16-V158 and CD16-F158 is measured using a BIAcore assay. Anti-CD20 IgG4 produced in H4-II-E cells shows a significantly higher affinity to both variants of the FcγRIIIa than anti-CD20 IgG4 produced in CHO cells.

### Example 11

### Anti-CD20 IgG1 Antibodies Produced in H4-II-E Rat Hepatoma Cells Activate In Vitro ADCC More Efficiently than Anti-CD20 IgG1 Antibodies Produced in CHO

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

ADCC assays are performed by the lactate dehydrogenase (LDH) release assay using as effector cells human peripheral blood mononuclear cells (PBMC). Aliquots of target tumor cells, the human Burkitt's lymphoma cell line Ramos, expressing human CD20 are distributed into 96-well U-bottomed plates (10.000 cells in 50 μl/well) and incubated with serial dilutions of the purified anti-CD20 IgG1 produced in H4-II-E cells or CHO cells (50 μL), respectively, in the presence of the PBMC (100 μL) at an E/T ratio of 20/1. After incubation for 4 h at 37° C., the supernatant LDH activity is measured. The percent specific cytolysis is calculated and is significantly higher if the anti-CD20 IgG1 used is produced and purified from the cell culture supernatant of H4-II-E cells than from CHO cells, indicating that H4-II-E produced IgG1 has a much higher potential to induce ADCC in effector cells (FIG. 10).

### Example 12

### Anti-HER2 IgG4 Antibodies Produced in H4-II-E Rat Hepatoma Cells Activate In Vitro ADCC More Efficiently than Anti-HER2 IgG4 Antibodies Produced in CHO

An anti-HER2 IgG4 antibody expression vector is used to stably transfect CHO cells and H4-II-E cells respectively. Stable anti-HER2 producing cell lines are isolated by selection for an antibiotic resistance marker and analysis of the cell supernatant of surviving cells for anti-HER2 expression by ELISA. Anti-HER2 producing cells are cultivated in a Serum-free Fed-Batch culture and recombinant antibodies are purified from the serum-free culture supernatant by Protein A-affinity chromatography using MabSelect™ (Amersham Biosciences).

ADCC assays are performed by the lactate dehydrogenase (LDH) release assay using as effector cells human peripheral blood mononuclear cells (PBMC). Aliquots of a HER2-positive breast cancer cell line are distributed into 96-well U-bottomed plates (10.000 cells in 50 μl/well) and incubated with serial dilutions of the purified anti-HER2 IgG4 produced in H4-II-E cells or CHO cells (50 μL), respectively, in the presence of the PBMC (100 μL) at an E/T ratio of 20/1. After incubation for 4 h at 37° C., the supernatant LDH activity is measured. The percent specific cytolysis is calculated and is significantly higher if the anti-HER2 IgG4 used is produced and purified from the cell culture supernatant of H4-II-E cells than from CHO cells, indicating that H4-II-E produced IgG4 has a much higher potential to induce ADCC in effector cells.

### Example 13

### Anti-CD20 IgG1 Antibodies Produced in H4-II-E Rat Hepatoma Cells Bind Components of the Complement System with Higher Affinity than Anti-CD20 IgG1 Antibodies Produced in CHO

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

The ability of each purified IgG1 to bind to the C1q component of the complement is studied by a flow cytometric assay using purified human complement C1q. Human Burkitt's lymphoma Ramos cells, expressing human CD20 are incubated with serial dilutions of anti-human CD20 IgG1 produced in H4-II-E cells or CHO respectively. After washing with PBS containing 1% (w/v) BSA, purified human complement C1q is added at a final concentration of 20 mg/mL and bound to the cell-bound IgG1 at 37° C. for 30 min Cells are then washed and incubated with fluorescein isothiocyanate-conjugated polyclonal antibodies against human C1q. Stained cells are analyzed by flow cytometry using FACSCalibur. Anti-CD20 IgG1 produced in H4-II-E cells shows a much stronger binding of the complement component C1q than Anti-CD20 IgG1 produced in CHO cells.

### Example 14

### Anti-CD20 IgG1 Antibodies Produced in H4-II-E Rat Hepatoma Cells Show an Enhanced Complement Activation In Vitro Compared to Anti-CD20 IgG1 Antibodies Produced in CHO Cells

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

CDC activity is determined by the WST-1 assay. Briefly, the target human Burkitt's lymphoma cell line Ramos, expressing human CD20, 2-fold diluted human serum complement (Sigma-Aldrich), and serial dilutions of anti-human CD20 produced in H4-II-E cells or CHO cells, respectively, are incubated in 96-well flat-bottomed plates (Greiner) for 1 h at 37° C. Cell proliferation WST-1 reagent is added to the wells (15 μL/well) and incubated for 6 h at 37° C. Absorbance in the wells is measured at 450 nm using a microplate reader (Tecan, Germany) and expressed in relative absorbance units (RAU) as an index of the viable cell number. The percent CDC is calculated according to the formula: CDC activity [%]=100*(RAU_{background}−RAUₜₑₛₜ)/RAU_{background}. CDC activity measured in the assay is significantly higher if anti-CD20 IgG1 is produced in H4-II-E and not in CHO cells.

### Example 15

### Anti-CD20 IgG1 Antibodies Produced in H4-II-E Rat Hepatoma Cells Bind the Neonatal Fc Receptor FcRn with Higher Affinity than Anti-CD20 IgG1 Antibodies Produced in CHO

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

To measure the binding of anti-CD20 IgG1 produced in H4-II-E cells or CHO cells to the neonatal Fc receptor FcRn, a BIAcore assay is used. A recombinant soluble human FcRn-b2 microglobulin complex is expressed in CHO/DG44 cells and purified from the culture supernatant by Ni-NTA chromatography (Qiagen). Antihuman b2-microglobulin monoclonal antibody (Abcam, Cambridge, UK) is immobilized onto the BIAcore T100 CM5 sensor chip using an amine-coupling kit (BIACORE). Soluble FcRn-b2 microglobulin complex is captured by the immobilized anti-b2-microglobulin antibody by injecting the complex. Each purified anti-CD20 IgG1 is diluted in HBS-EP+ buffer (0.01 M HEPES, 0.15M NaCl, 3 mM EDTA, 0.05% Surfactant P20) whose pH is adjusted to 6.0 at five different concentrations (from 4.17 to 66.7 nM), and each diluted IgG1 is injected over the complex-captured sensor surface or blank at a flow rate of 5 mL/min Soluble FcRn and IgG1 bound to the sensor surface are removed by injecting 7.5 mM HCl at a flow rate of 60 mL/min for 1 min. The experiments are performed at 25° C. with HBS-EP+ as a running buffer. The data obtained by blank subtraction are used for the data analysis. An apparent association rate constant (ka), a dissociation rate constant (kd), and the binding affinity (KD) are calculated by the bivalent fitting model using BIAcore T100 evaluation software. Anti-CD20 IgG1 produced in H4-II-E cells shows a much stronger binding of the neonatal Fc receptor FcRn than anti-CD20 IgG1 produced in CHO cells (FIG. 11).

### Example 16

### A MCP1-Fc-Fusion Protein Produced in H4-II-E Rat Hepatoma Cells Binds to the Fc Receptors CD16-V158 and CD16-F158 (FCGRIIIA) With Higher Affinity than MCP1-Fc-Fusion Proteins Produced in CHO

An MCP1-Fc fusion protein expression vector is used to stably transfect CHO cells and H4-II-E cells respectively. MCP1-Fc fusion protein producing H4-II-E cells are generated by transfection with DNA constructs encoding SEQ ID NO:6. Stable MCP1-Fc fusion protein producing cell lines are isolated by selection for an antibiotic resistance marker and analysis of the cell supernatant of surviving cells for MCP1-Fc expression by ELISA. MCP1-Fc producing cells are cultivated in a Serum-free Fed-Batch culture and recombinant antibodies are purified from the serum-free culture supernatant by Protein A-affinity chromatography using MabSelect™ (Amersham Biosciences).

The binding kinetics of MCP1-Fc produced in H4-II-E cells and in CHO cells to FcγRIIIa receptors CD16-V158 and CD16-F158 is measured using a BIAcore assay. MCP1-Fc produced in H4-II-E cells shows a significantly higher affinity to both variants of the FcγRIIIa than anti-CD20 IgG1 produced in CHO cells.

### Example 17

### A Fc-Fusion Protein Comprising EPO-Fc Produced in H4-II-E Rat Hepatoma Cells Binds to the Fc Receptors CD16-V158 and CD16-F158 (FCGRIIIA) With Higher Affinity than Fc-Fusion Proteins Comprising EPO-Fc Produced in CHO

An expression vector comprising the nucleic acid sequence of EPO fused in frame to the nucleic acid sequence of IgG1 Fc, is used to stably transfect CHO cells and H4-II-E cells respectively. EPO-Fc fusion protein producing H4-II-E cells are generated by transfection with DNA constructs encoding SEQ ID NO:7. Stable Fc fusion protein comprising EPO-Fc producing cell lines are isolated by selection for an antibiotic resistance marker and analysis of the cell supernatant of surviving cells for Fc-fusion protein expression by ELISA. Fc-fusion protein producing cells are cultivated in a Serum-free Fed-Batch culture and EPO-Fc proteins are purified from the serum-free culture supernatant by Protein A-affinity chromatography using MabSelect™ (Amersham Biosciences).

The binding kinetics of the Fc fusion protein comprising EPO-Fc produced in H4-II-E cells and in CHO cells to FcγRIIIa receptors CD16-V158 and CD16-F158 is measured using a BIAcore assay. Fc fusion protein comprising EPO-Fc produced in H4-II-E cells shows a significantly higher affinity to both variants of the FcγRIIIa than anti-CD20 IgG1 produced in CHO cells.

### Example 18

### Anti-CD20 IgG1 Antibodies Produced in H4-II-E Rat Hepatoma Cells Bind the Fc Receptors CD32A and CD32B with Lower Affinity than Anti-CD20 IgG1 Antibodies Produced in CHO

An anti-CD20 IgG1 antibody expression vector is used to stably transfect CHO cells and H4-II-E cells respectively. Anti-CD20 IgG1 antibody producing H4-II-E cells are generated by transfection with DNA constructs encoding the heavy chain (SEQ ID NO:2) and light chain (SEQ ID NO:3) of the anti-CD20 IgG1 antibody. Stable anti-CD20 producing cell lines are isolated by selection for an antibiotic resistance marker and analysis of the cell supernatant of surviving cells for anti-CD20 expression by ELISA. Anti-CD20 producing cells are cultivated in a Serum-free Fed-Batch culture and recombinant antibodies are purified from the serum-free culture supernatant by Protein A-affinity chromatography using MabSelect™ (Amersham Biosciences).

The binding kinetics of anti-CD20 IgG1 produced in H4-II-E cells and in CHO cells to FcγRIIa and FcγRIIb receptors is measured using a BIAcore assay. Anti-CD20 IgG1 produced in H4-II-E cells shows a significantly higher affinity to both variants of the FcγRIIa and FcγRIIb than anti-CD20 IgG1 produced in CHO cells.

### REFERENCE LIST

1. Burton, D. R. and Dwek, R. A. (2006). Immunology. Sugar determines antibody activity. Science 313, 627-628.
2. Edelman, G. M., Cunningham, B. A., Gall, W. E., Gottlieb, P. D., Rutishauser, U., and Waxdal, M. J. (1969). The covalent structure of an entire gammaG immunoglobulin molecule. Proc. Natl. Acad. Sci. U.S.A 63, 78-85.
3. Ellison, J. and Hood, L. (1982). Linkage and sequence homology of two human immunoglobulin gamma heavy chain constant region genes. Proc. Natl. Acad. Sci. U.S.A 79, 1984-1988.
4. Fallaux, F. J., Bout, A., van, d. V., I, van den Wollenberg, D. J., Hehir, K. M., Keegan, J., Auger, C., Cramer, S. J., van, O. H., van der Eb, A. J., Valerio, D., and Hoeben, R. C. (1998). New helper cells and matched early region 1-deleted adenovirus vectors prevent generation of replication-competent adenoviruses. Hum. Gene Ther. 9, 1909-1917.
5. Hamako, J., Matsui, T., Ozeki, Y., Mizuochi, T., and Titani, K. (1993). Comparative studies of asparagine-linked sugar chains of immunoglobulin G from eleven mammalian species. Comp Biochem. Physiol B 106, 949-954.
6. Horikoshi, N., Tashiro, F., Tanaka, N., and Ueno, Y. (1988). Modulation of hormonal induction of tyrosine aminotransferase and glucocorticoid receptors by aflatoxin B1 and sterigmatocystin in Reuber hepatoma cells. Cancer Res. 48, 5188-5192.
7. Houser, W. H., Raha, A., and Vickers, M. (1992). Induction of CYP1A1 gene expression in H4-II-E rat hepatoma cells by benzo[e]pyrene. Mol. Carcinog. 5, 232-237.
8. Jenkins, N., Parekh, R. B., and James, D. C. (1996). Getting the glycosylation right: implications for the biotechnology industry. Nat. Biotechnol. 14, 975-981.
9. Kabat, E. A. (1988). Antibody complementarity and antibody structure. J. Immunol. 141, S25-S36.
10. Kabat, E. A., Wu, T. T., Perry, H. M., Gottesman, K. S., and Foeller, C. (1991). Sequences of Proteins of Immunological Interest. U.S. Department of Health and Human Services, Natl. Inst. of Health, Bethesda.
11. Kanda, Y., Yamane-Ohnuki, N., Sakai, N., Yamano, K., Nakano, R., Inoue, M., Misaka, H., Iida, S., Wakitani, M., Konno, Y., Yano, K., Shitara, K., Hosoi, S., and Satoh, M. (2006). Comparison of cell lines for stable production of fucose-negative antibodies with enhanced ADCC. Biotechnol. Bioeng. 94, 680-688.
12. Kaufman, R. J. (1990). Selection and coamplification of heterologous genes in mammalian cells. Methods Enzymol. 185, 537-566.
13. Lifely, M. R., Hale, C., Boyce, S., Keen, M. J., and Phillips, J. (1995). Glycosylation and biological activity of CAMPATH-1H expressed in different cell lines and grown under different culture conditions. Glycobiology 5, 813-822.
14. Miyazaki, M., Bai, L., Taga, H., Hirai, H., Sato, J., and Namba, M. (1991). Expression of liver-specific functions and secretion of a hepatocyte growth factor by a newly established rat hepatoma cell line growing in a chemically-defined serum-free medium. Res. Exp. Med. (Berl) 191, 297-307.
15. Mori, K., Kuni-Kamochi, R., Yamane-Ohnuki, N., Wakitani, M., Yamano, K., Imai, H., Kanda, Y., Niwa, R., Iida, S., Uchida, K., Shitara, K., and Satoh, M. (2004). Engineering Chinese hamster ovary cells to maximize effector function of produced antibodies using FUT8 siRNA. Biotechnol. Bioeng. 88, 901-908.
16. Niwa, A., Yamamoto, K., Sorimachi, K., and Yasumura, Y. (1980). Continuous culture of Reuber hepatoma cells in serum-free arginine-, glutamine- and tyrosine-deprived chemically defined medium. In Vitro 16, 987-993.
17. Noguchi, A., Mukuria, C. J., Suzuki, E., and Naiki, M. (1995) Immunogenicity of N-glycolylneuraminic acid-containing carbohydrate chains of recombinant human erythropoietin expressed in Chinese hamster ovary cells. J. Biochem. 117, 59-62.
18. PITOT, H. C., PERAINO, C., MORSE, P. A., Jr., and POTTER, V. R. (1964). HEPATOMAS IN TISSUE CULTURE COMPARED WITH ADAPTING LIVER IN VIVO. Natl. Cancer Inst. Monogr 13, 229-245.
19. Raju, T. S., Briggs, J. B., Borge, S. M., and Jones, A. J. (2000). Species-specific variation in glycosylation of IgG: evidence for the species-specific sialylation and branch-specific galactosylation and importance for engineering recombinant glycoprotein therapeutics. Glycobiology 10, 477-486.
20. REUBER, M. D. (1961). A transplantable bile-secreting hepatocellular carcinoma in the rat. J. Natl. Cancer Inst. 26, 891-899.
21. Ripka, J., Adamany, A., and Stanley, P. (1986). Two Chinese hamster ovary glycosylation mutants affected in the conversion of GDP-mannose to GDP-fucose. Arch. Biochem. Biophys. 249, 533-545.
22. Sambrook, J., Fritsch, d. F., and Maniatis, T. (1989). Molecular Cloning: A Laboratory Manual. (Cold Spring Harbor: Cold Spring Harbor Laboratory Press).
23. Scallon, B. J., Tam, S. H., McCarthy, S. G., Cai, A. N., and Raju, T. S. (2007). Higher levels of sialylated Fc glycans in immunoglobulin G molecules can adversely impact functionality. Mol. Immunol. 44, 1524-1534.
24. Shields, R. L., Lai, J., Keck, R., O'Connell, L. Y., Hong, K., Meng, Y. G., Weikert, S. H., and Presta, L. G. (2002). Lack of fucose on human IgG1 N-linked oligosaccharide improves binding to human Fcgamma RIII and antibody-dependent cellular toxicity. J. Biol. Chem. 277, 26733-26740.
25. Shinkawa, T., Nakamura, K., Yamane, N., Shoji-Hosaka, E., Kanda, Y., Sakurada, M., Uchida, K., Anazawa, H., Satoh, M., Yamasaki, M., Hanai, N., and Shitara, K. (2003). The absence of fucose but not the presence of galactose or bisecting N-acetylglucosamine of human IgG1 complex-type oligosaccharides shows the critical role of enhancing antibody-dependent cellular cytotoxicity. J. Biol. Chem. 278, 3466-3473.
26. Umana, P., Jean-Mairet, J., and Bailey, J. E. (1999). Tetracycline-regulated overexpression of glycosyltransferases in Chinese hamster ovary cells. Biotechnol. Bioeng. 65, 542-549.
27. Urlaub, G. and Chasin, L. A. (1980). Isolation of Chinese hamster cell mutants deficient in dihydrofolate reductase activity. Proc. Natl. Acad. Sci. U.S.A 77, 4216-4220.
28. Urlaub, G., Kas, E., Carothers, A. M., and Chasin, L. A. (1983). Deletion of the diploid dihydrofolate reductase locus from cultured mammalian cells. Cell 33, 405-412.
29. Walsh, G. and Jefferis, R. (2006). Post-translational modifications in the context of therapeutic proteins. Nat. Biotechnol. 24, 1241-1252.
30. Werner, R. G. (2004). Economic aspects of commercial manufacture of biopharmaceuticals. J. Biotechnol. 113, 171-182.
31. Wright, A., Sato, Y., Okada, T., Chang, K., Endo, T., and Morrison, S. (2000). In vivo trafficking and catabolism of IgG1 antibodies with Fc associated carbohydrates of differing structure. Glycobiology 10, 1347-1355.
32. Yamane-Ohnuki, N., Kinoshita, S., Inoue-Urakubo, M., Kusunoki, M., Iida, S., Nakano, R., Wakitani, M., Niwa, R., Sakurada, M., Uchida, K., Shitara, K., and Satoh, M. (2004). Establishment of FUT8 knockout Chinese hamster ovary cells: an ideal host cell line for producing completely defucosylated antibodies with enhanced antibody-dependent cellular cytotoxicity. Biotechnol. Bioeng. 87, 614-622.

1. A rat hepatoma cell comprising a nucleic acid sequence encoding an antibody or Fc-fusion protein, wherein said nucleic acid sequence is operatively linked to at least one regulatory sequence allowing for expression of said nucleic acid sequence encoding an antibody or Fc-fusion protein.

2. The rat hepatoma cell of claim 1, wherein said cell is a H4-II-E cell.

3. The rat hepatoma cell of claim 1, wherein said cell is:
a) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
b) a cell which is deposited with the European Collection of Cell Cultures under the number ECACC, Cat. no. 87031301 or the American Type Culture Collection ATCC under the deposit no. CRL-1548, or
c) a cell which is deposited with the DSMZ (Deutsche Sammlung von Mikroorganismen und Zellkulturen GmbH) under the accession number DSM ACC3129 (H4-II-E), or
d) a cell which is deposited with the DSMZ (Deutsche Sammlung von Mikroorganismen und Zellkulturen GmbH) under the accession number DSM ACC3130 (H4-II-Es), or
e) a derivative or progeny of any one cell of a) or b) or c) or d).

4. The rat hepatoma cell according to claim 3, wherein said cell is a cell deposited with the DSMZ (Deutsche Sammlung von Mikroorganismen und Zellkulturen GmbH) under the accession number DSM ACC3129 (H4-II-E) or wherein said cell is a cell deposited with the DSMZ (Deutsche Sammlung von Mikroorganismen and Zellkulturen GmbH) under the accession number DSM ACC3130 (H4-II-Es).

5. The rat hepatoma cell according to claim 1, further characterized in that
a) the degree of glycosidic structures contained in the antibody or Fc-fusion protein expressed by said cell, which contain fucose, is less than 20%, 10% or 5% or
b) the degree of glycosidic structures contained in said antibody or Fc-fusion protein expressed by said cell, which contain fucose, ranges between 0% to 20%, 0% to 10%, 0% to 5%, 0.5% to 20%, 0.5% to 10%, 0.5% to 5%, 1% to 20%, 1% to 10% or 1% to 5%.

6. The rat hepatoma cell according to claim 1, further characterized in that
a) the degree of glycosidic structures contained in said antibody or Fc-fusion protein expressed by said cell, which contain at least one galactose residue, is more than 40%, 45% or 50% or
b) the degree of glycosidic structures contained in said antibody or Fc-fusion protein expressed by said cell, which contain at least one galactose residue ranges between 40% to 100%, 45% to 100%, 50% to 100%, 51% to 100%, 40% to 99.5%, 45% to 99.5%, 50% to 99.5% or 51% to 99.5%, 40% to 99%, 45% to 99%, 50% to 99% or 51% to 99%.

7. The rat hepatoma cell according to claim 6, wherein said glycosidic structures contain one or two galactose residues (G1 or G2), optionally linked to N-acetylglucosamine (GlcNAc) at the terminal non-reducing end of said glycosidic structures.

8. The rat hepatoma cell according to claim 1, further characterized in that
a) the degree of glycosidic structures contained in said antibody or Fc-fusion protein expressed by said cell, which contain terminal sialic acid or neuraminic acid residues, is more than 5% or more than 10% or
b) the degree of glycosidic structures contained in said antibody or Fc-fusion protein expressed by said cell, which contain terminal sialic acid or neuraminic acid residues, ranges between 0-8%, 1-8%, 5-10%, 10-50% or 10-45%.

9. The rat hepatoma cell according to claim 1, further characterized by carrying a selection marker gene such as neomycin-phosphotransferase (NPT), resistance genes against puromycin, hygromycin or zeocin or an amplifyable selection marker gene such as dihydrofolate reductase (DHFR) or glutamine synthetase (GS).

10. The rat hepatoma cell according to claim 1, wherein said regulatory sequence allowing for expression of said nucleic acid sequence encoding an antibody or Fc-fusion protein is
a) a promoter, or
b) an enhancer, or
c) a 5′-UTR sequence.

11. The rat hepatoma cell according to claim 1, wherein said antibody or Fc fusion protein contains a glycosidic structure linked to an N-Asparagine (N-Asn) residue, wherein said glycosidic structure comprises the following sugar chain:

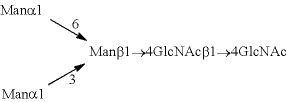

12. The rat hepatoma cell according to claim 1, wherein said antibody or Fc fusion protein contains a glycosidic structure linked to an N-Asparagine (N-Asn) residue, wherein said glycosidic structure comprises the following sugar chain:

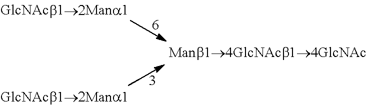

13. The rat hepatoma cell according to claim 11 or 12, wherein said N-Asn is preferably N-Asn (297) according to the Kabat EU nomenclature.

14. The rat hepatoma cell of claim 1, wherein said cell is adapted to growth in serum-free and calcium-reduced or preferably calcium-free medium.

15. The rat hepatoma cell according to claim 1, wherein said cell is adapted to growth in suspension culture.

16. The rat hepatoma cell of claim 1, wherein said cell has low sensitivity to apoptosis and/or high robustness towards cellular stresses in comparison to YB2/0 cells.

17. A method for producing a glycoprotein of interest comprising the steps of:
a) providing a rat hepatoma cell,
b) optionally adapting said cell of step a) to growth in suspension culture,
c) optionally adapting said cell of step a) and/or step b) to growth in serum-free medium,
d) optionally adapting said cell of step a) and/or step b) and/or step c) to growth in calcium-reduced or calcium-free medium,
e) transfecting this optionally adapted rat hepatoma cell with a nucleic acid sequence encoding a recombinant glycoprotein of interest,
f) cultivating said transfected cell of step e) under conditions which allow expression of said glycoprotein of interest, and
g) optionally isolating and purifying said glycoprotein of interest.

18. The method of claim 17, wherein said rat hepatoma cell is a H4-II-E cell, or said cell is:
a) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
b) a cell which is deposited with the European Collection of Cell Cultures under the number ECACC, Cat. no. 87031301 or the American Type Culture Collection ATCC under the deposit no. CRL-1548 or
c) a cell which is deposited with the DSMZ (Deutsche Sammlung von Mikroorganismen und Zellkulturen GmbH) under the accession number DSM ACC3129 (H4-II-E), or
d) a cell which is deposited with the DSMZ (Deutsche Sammlung von Mikroorganismen und Zellkulturen GmbH) under the accession number DSM ACC3130 (H4-II-Es), or
e) a derivative or progeny of any one cell of a) or b) or c) or d).

19. The method according to claim 18, wherein said cell is a cell deposited with the DSMZ (Deutsche Sammlung von Mikroorganismen und Zellkulturen GmbH) under the accession number DSM ACC3129 (H4-II-E) or a cell deposited with the DSMZ (Deutsche Sammlung von Mikroorganismen und Zellkulturen GmbH) under the accession number DSM ACC3130 (H4-II-Es).

20. The method of claim 17, wherein said medium of step b), c) and/or d) is additionally free of any protein/peptide of animal origin.

21. A method according to claim 17, further characterized in that the transfection step e) comprises introducing an expression vector comprising a nucleic acid sequence encoding for said glycoprotein of interest operatively linked to at least one regulatory sequence allowing for expression of said nucleic acid sequence encoding a glycoprotein of interest into said rat hepatoma cell.

22. The method according to claim 17, wherein said glycoprotein of interest is an antibody or Fc-fusion protein, wherein the antibody or Fc-fusion protein has
a) FcγRIIIa binding activity and preferably ADCC, or
b) complement binding activity and preferably CDC, or
c) binding activity to the neonatal Fc receptor FcRn and preferably serum stability.

23. A method for producing a (recombinant) antibody or Fc-fusion protein having
a) FcγRIIIa binding activity and/or
b) complement binding activity and/or
c) binding activity of the neonatal Fc receptor FcRn,
comprising producing said antibody or Fc fusion protein in a rat hepatoma cell, wherein said rat hepatoma cell is preferably a H4-II-E cell, or said cell is:
i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548, or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

24. The method according to claim 23, wherein said cell is a cell deposited with the DSMZ (Deutsche Sammlung von Mikroorganismen und Zellkulturen GmbH) under the accession number DSM ACC3129 (H4-II-E) or a cell deposited with the DSMZ (Deutsche Sammlung von Mikroorganismen und Zellkulturen GmbH) under the accession number DSM ACC3130 (H4-II-Es).

25. A method according to claim 23, wherein
i) said antibody or Fc fusion protein of claim 23 a) has antibody dependent cellular cytotoxicity (ADCC) or
ii) said antibody or Fc fusion protein of claim 23 b) has complement dependent cytotoxicity (CDC) or
iii) said antibody or Fc fusion protein of claim 23 c) has serum stability.

26. A method of generating a host cell for production of recombinant glycoprotein comprising:
a) providing a rat hepatoma cell,
b) adapting said rat hepatoma cell of step a) to growth in suspension culture, and
c) adapting said rat hepatoma cell of step a) to growth in serum-free medium, and
d) adapting said rat hepatoma cell of step a) to growth in calcium-reduced or calcium-free medium, and
e) optionally adapting said rat hepatoma cell of step a) to growth in medium free of any protein/peptide of animal origin,
f) optionally selecting a single cell clone, and
g) obtaining a host cell.

27. The method of claim 26, wherein said rat hepatoma cell is a H4-II-E cell, preferably said cell is:
i) a cell derived from a cell selected from the group consisting of: European Collection of Cell Cultures (ECACC, Cat. no. 87031301), American Type Culture Collection (ATCC, deposit no. CRL-1548), H4-II-E-C3 cell line (CRL-1600 or HPACC No. 85061112 or ECACC catalogue no. 85061112), H4II cell line (HPACC Nr. 89042702), H4-TG cell line (CRL-1578), H5 cell line (HPACC, Nr. 94101905) and H4-S cell line (HPACC Nr. 89102001), or
ii) a cell which is deposited with the European Collection of Cell Cultures under the number ECACC, Cat. no. 87031301 or the American Type Culture Collection ATCC under the deposit no. CRL-1548, or
iii) a cell which is deposited with the DSMZ (Deutsche Sammlung von Mikroorganismen und Zellkulturen GmbH) under the accession number DSM ACC3129 (H4-II-E), or
iv) a cell which is deposited with the DSMZ (Deutsche Sammlung von Mikroorganismen und Zellkulturen GmbH) under the accession number DSM ACC3130 (H4-II-Es), or
v) a derivative or progeny of any one cell of i) or ii) or iii) or iv).

28. The method according to claim 27, wherein said cell is a cell deposited with the DSMZ (Deutsche Sammlung von Mikroorganismen und Zellkulturen GmbH) under the accession number DSM ACC3129 (H4-II-E) or a cell deposited with the DSMZ (Deutsche Sammlung von Mikroorganismen und Zellkulturen GmbH) under the accession number DSM ACC3130 (H4-II-Es).

29. The method of claim 26 further comprising
h) transfecting said obtained host cell of step g) of claim 26 with a nucleic acid sequence encoding a glycoprotein of interest, and
i) optionally cultivating said transfected cell of step h) under conditions which allow expression of said glycoprotein of interest.

30. The method according to claim 29, wherein said glycoprotein of interest is an antibody or Fc fusion protein, or an antibody or Fc fusion protein having ADCC and/or CDC and/or serum stability.

31. A cell generated according to claim 26.

32. A (rat hepatoma) cell deposited with the DSMZ (Deutsche Sammlung von Mikroorganismen und Zellkulturen GmbH) under the accession number DSM ACC3130 (H4-II-Es).

33. Use of a rat hepatoma cell according to claim 1 or 31 as a host cell for biopharmaceutical production.
